(19) United States
(12) Patent Application Publication
(10) Pub. No.: US 2026/0265792 A1
(43) Pub. Date: Sep. 10, 2026

(54) ENGINEERED OBLIGATE FERMENTING BACTERIA FOR THE FERMENTATIVE PRODUCTION OF D-LACTATE AND ISOBUTANOL

(71) Applicant: Max-Planck-Gesellschaft zur Förderung der Wissenschaften e.V., Munich (DE)

(72) Inventors: Steffen Nikolaus Lindner-Mehlich, Berlin (DE); Jan Lukas Krusemann, Berlin (DE); Eleni Mavrothalassiti, 2th. Kobenhavn V (DK); Helena Anna Maria Schulz-Mirbach, Amoneburg (DE); Theofania Pagona Andreadaki, Newcastle Upon Tyne (GB)

(21) Appl. No.: 19/163,409

(22) PCT Filed: Mar. 11, 2024
(86) PCT No.: PCT/EP2024/056452

§ 371 Date: Sep. 9, 2025

(30) Foreign Application Priority Data
Mar. 10, 2023 (EP) 23161350.6
Nov. 30, 2023 (EP) 23213564.0

(51) Int. Cl. C12P 7/56; C12N 1/20; C12P 7/06; C12P 7/16; C12R 1/19

(52) U.S. Cl. CPC C12P 7/56; C12N 1/20; C12P 7/065; C12P 7/16; C12R 2001/19

(57) ABSTRACT

The present invention relates to genetically engineered bacteria comprising a deletion of ndh gene; a deletion of one or more nuo genes; a deletion of dld gene, wherein the one or more nuo genes are selected from nuoA, nuoB, nuoC, nuoD, nuoE, nuoF, nuoG, nuoH, nuoI, nuoJ, nuoK, nuoL, nuoM and nuoN; wherein the genetically engineered bacterium is able of oxygen uptake, i.e. the genetically engineered bacterium is able to use oxygen as electron acceptor, and wherein the bacteria is genetically engineered to produce a fermentation product in presence of oxygen. In particular, the present invention provides bacteria genetically engineered to produce lactate from glycerol or from glucose in presence of oxygen. The present invention further provides bacteria genetically engineered to produce isobutanol and/or ethanol from glycerol in presence of oxygen. Further described is a method for producing a fermentation product using the disclosed genetically engineered bacteria.

The present invention relates to genetically engineered bacterium comprising a deletion of ndh gene; a deletion of one or more nuo genes; and a deletion of dld gene, wherein the one or more nuo genes are selected from nuoA, nuoB, nuoC, nuoD, nuoE, nuoF, nuoG, nuoH, nuoI, nuoJ, nuoK, nuoL, nuoM and nuoN; wherein the genetically engineered bacteria is able of oxygen uptake, i.e. the genetically engineered bacteria is able to use oxygen as electron acceptor, and wherein the bacteria are genetically engineered to produce a fermentation product in presence of a terminal electron acceptor, particularly in presence of oxygen. In particular, the present invention provides bacteria genetically engineered to produce lactate or isobutanol from glycerol or from glucose in presence of a terminal electron acceptor. Thus, the present invention particularly provides bacteria genetically engineered to produce lactate or isobutanol from glycerol or from glucose in presence of oxygen. The present invention further provides bacteria genetically engineered to produce isobutanol and/or ethanol from glycerol in presence of a terminal electron acceptor. Thus, the present invention particularly further provides bacteria genetically engineered to produce isobutanol and/or ethanol from glycerol in presence of oxygen. Further described is a method for producing a fermentation product using the disclosed genetically engineered bacteria.

### BACKGROUND OF THE INVENTION

Facultative anaerobe bacteria can grow both under aerobic or anaerobic conditions by using carbohydrates as sole carbon and energy source. In the glycolysis pathway, glucose is transported into the cell and phosphorylated to glucose-6-phosphate either by phosphoenolpyruvate dependent phosphotransferase system or by ATP dependent kinases. Then glucose-6-phosphate is metabolized to two molecules of pyruvate, two adenosine triphosphates (ATP) and two nicotinamide adenine dinucleotides (NADH). The two NADH generated by the glycolysis pathway are then re-oxidized to NAD⁺.

Under aerobic conditions, thus in the presence of oxygen, the NADH generated during glycolysis is re-oxidized in the respiratory chain and pyruvate is converted to acetyl-CoA and CO₂ by the pyruvate dehydrogenase complex. Acetyl-CoA is then further processed within the citric acid cycle. The aerobic respiratory chain of E. coli involves ubiquinone (Q) mediated electron transfer from dehydrogenases to cytochrome oxidases. NADH dehydrogenase I (NDH-1) and NADH dehydrogenase II (NDH-2) are two NADH dehydrogenases in E. coli that oxidize NADH to NAD⁺: Both NADH dehydrogenases donate electrons to the cytochrome oxidases to reduce O₂ to H₂O. The three cytochrome oxidases in E. coli are cytochrome bo oxidase (CyoABCD), cytochrome bd-I oxidase (CydAB) and cytochrome bd-II oxidase (AppBC).

The anaerobic respiratory chain of bacteria involves menaquinone (MQ) mediated electron transfer from dehydrogenases to electron accepting terminal reductases. Anaerobic electron donors include NADH and NADH dehydrogenase I (NDH-1) is also expressed under anaerobic conditions. Anaerobic electron acceptors include fumarate, nitrate, nitrite, trimethylamine-N-oxide, and dimethylsulfoxide.

Under anaerobic conditions and in absence of alternative electron acceptors such as fumarate or nitrate, the NADH oxidation steps are accomplished by reduction of several intermediates e.g. pyruvate. A so called “mixed-acid fermentation” can be used to produce lactate, succinate, ethanol, acetate, formate, carbon dioxide, and hydrogen from pyruvate. To achieve a proper fermentation balance, the amount of NADH produced must match the amount of NADH consumed. In general, a mixture of ethanol, lactate and acetate is used, all of which consume different amounts of NADH.

Several engineered bacteria strains are known from the prior art that have been modified with the aim of directing the fermentation pathways to a specific fermentation product, e.g. D-lactate, under anaerobic conditions. The usual strategy for providing such engineered strains is to eliminate the fermentation pathways that lead to the production of undesired fermentation products, which is usually achieved by a deletion of the genes encoding the enzymes expressed under anaerobic conditions that catalyze the reactions to the undesired fermentation products. Overexpression of pathways involved in the conversion of the carbon source, e.g. glucose, can also be used to direct the fermentation pathway to a specific fermentation product, e.g. D-lactate. Other engineered bacteria strains are known from the prior art that have been modified to allow the use of alternative carbon sources for the production of specific fermentation end products under anaerobic or micro-aerobic conditions. For example, the use of xylose, sucrose, or glycerol as carbon sources instead of glucose for the production of D-lactate under anaerobic conditions or under micro-aerobic conditions has been described for a few engineered E. coli strains. However, the above described E. coli strains particularly require strict maintenance of anaerobic or micro-aerobic conditions for the production of D-lactate via fermentation. Thereby, micro-aerobic conditions are even harder to control and adjust in large bioreactors. This represents a major drawback for the use of engineered bacteria strains for the production of D-lactate via fermentation.

The US patent application US 2012/0064581 A1 discloses engineered E. coli strains having a deletion of the cyoABCD genes, cydAB genes and cbdAD genes encoding the three cytochrome oxidases in E. coli cytochrome bo oxidase (cyoABCD), cytochrome bd-I oxidase (cydAB) and cytochrome bd-II oxidase (appBC). These engineered E. coli strains have been further used as platform strains in evolutionary engineering to provide E. coli strains with the ability to convert glucose to D-lactate under one or both aerobic and anaerobic conditions. The adaptive evolution of these strains resulted in E. coli strains having strong decrease or complete elimination of oxygen uptake. As the oxygen uptake is severely limited, the anaerobic pathways responsible for mixed-acid fermentation is also active under aerobic conditions. These E. coli strains are thus suitable for the production of fermentation products, e.g. D-lactate from usual carbon sources such as glucose under “aerobic conditions”. However, the use of alternative carbon sources such as glycerol for the production of D-lactate or the exploitation of alternative fermentation pathways is not possible with these E. coli strains.

A further drawback associated with the E. coli strains of US patent application US 2012/0064581 A1 and with other engineered bacteria from the prior art, such as the bacteria using glycerol as a carbon source instead of glucose for the production of D-lactate under micro-aerobic conditions, is that the initially genetically modified bacteria had to undergo further adaptive evolution to ultimately obtain bacteria with the desired fermentative properties. The use of adaptive evolution has particular drawbacks with regard to the reproducibility and re-engineering of these bacteria. Engineered bacteria that can be obtained by targeted genetic manipulation already having the desired properties without further adaptive evolution are therefore particularly desirable. In addition, it is particularly desirable that engineered bacteria remain stable over time against undesirable evolutionary adaptations and mutations when used in accordance with their intended purpose, e.g. in the production of D-lactate from glucose or alternative carbon sources as glycerol.

Fengming L. et al., Plos One 2013, 8(10): e78595 disclose engineered E. coli strains for improving fatty acid production having at least a deletion of cyoA gene. Thus, the use of alternative carbon sources such as glycerol for the production of D-lactate or the exploitation of alternative fermentation pathways is also not possible with these E. coli strains.

In the prior art, the production of chemicals and fuels via microbial fermentation has been largely based on the use of sugars such as glucose, lactose, xylose, etc. as carbon source. Biodiesel is produced by a transesterification reaction using vegetable oils or animal fats and an alcohol, a process that inevitably generates large amounts of glycerol as by-product. It is expected that the production of biodiesel will continue to increase in the future. The further utilization of glycerol produced in biodiesel production is therefore reasonable and desirable, in particular, the conversion of glycerol into higher value products e.g. D-lactate. Glycerol has a high degree of reduction which is advantageous for the microbial production of biofuels and other reduced chemicals compared to glucose. However, only a few bacterial species are known to grow on glycerol under strictly anaerobic conditions without any external electron acceptor. The potential use of glycerol as carbon source in fermentation processes in bacteria is limited by the inability of bacteria to ferment glycerol in the absence of external electron acceptor. It has been extensively investigated whether and under which conditions bacteria can anaerobically grow on glycerol as sole substrate and convert it into ethanol or other valuable products. However, the solutions proposed in the prior art are still insufficient.

Thus, there is a need for engineered bacteria that can be used for the production of specific fermentation products, in particular D-lactate, under aerobic conditions and further for engineered bacteria that allow the conversion of glycerol into higher value products, such as D-lactate, isobutanol, and ethanol.

It is the objective of the present invention to provide genetically engineered bacteria for the production of specific fermentation products, in particular D-lactate, isobutanol, and ethanol under aerobic conditions. A further objective of the present invention is to provide a genetically engineered bacteria for the production of specific fermentation products, in particular D-lactate, isobutanol, and ethanol under aerobic conditions and from alternative carbon sources such as glycerol. Moreover, it is a further objective of the present invention to provide a genetically engineered bacteria that does not require adaptive evolution techniques for reproduction.

The objective of the present invention is solved by the teaching of the independent claims. Further advantageous features, aspects and details of the invention are evident from the dependent claims, the description, the figures, and the examples of the present application.

### BRIEF DESCRIPTION OF THE INVENTION

The present invention relates to a genetically engineered bacterium comprising:
a deletion of ndh gene;
a deletion of one or more nuo genes;
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor, and
   wherein the bacterium is genetically engineered to produce a fermentation product in presence of a terminal electron acceptor, preferably in presence of oxygen. Thus, the genetically engineered bacterium produces a fermentation product from a carbon source in presence of a terminal electron acceptor, preferably in presence of oxygen.

Importantly, the genetically engineered bacterium, bacteria or bacterial strains disclosed herein do not comprise a deletion of cyoABCD genes, cydAB genes and/or cbdAD genes.

In further preferred embodiments, the one or more nuo genes are selected from nuoE gene, nuoF gene and/or nuoG gene, and more preferably the one or more nuo genes are nuoE gene, nuoF gene, and nuoG gene, i.e. the genetically engineered bacterium comprises a deletion of nuoEFG genes.

The genetically engineered bacterium of the present invention provides an advantageous platform enabling fermentation processes that can take place in spite of the presence of oxygen, i.e. under aerobic conditions, thereby allowing the use of alternative carbon sources such as glycerol for the production of specific fermentation products, in particular D-lactate, isobutanol and/or ethanol.

Moreover, the genetically engineered bacterium of the present invention can be advantageously used as a platform strain for further deletion of one or more additional genes to improve the selective production of specific fermentation end products, in particular of D-lactate, isobutanol and/or ethanol, and to prevent the possible production of undesired fermentation by-products. Moreover, it has been found that further deletions of one or more additional genes can be beneficial for provision of genetically engineered bacteria that are more stable and robust against undesired evolutionary adaptations and mutations.

Therefore, in preferred embodiments, the genetically engineered bacterium further comprises a deletion of mqo gene. In more preferred embodiments, the genetically engineered bacterium further comprises a deletion of gpsA gene. In still more preferred embodiments, the genetically engineered bacterium further comprises a deletion of poxB gene and/or a deletion of sdhABCD genes. In further more preferred embodiments, the genetically engineered bacterium further comprises a deletion of one or more genes selected from the group comprising glcDEF genes, lhgO gene, putA gene, lldD gene and/or dadA gene. In further more preferred embodiments, the genetically engineered bacterium further comprises a deletion of one or more genes selected from the group comprising kefF gene, mdaB-ygiN genes, glpABC genes, wrbA gene, yieF gene, and fadE gene.

Thus, in preferred embodiments the genetically engineered bacterium comprises a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and further comprises a deletion of mqo gene.

Thus, in further preferred embodiments the genetically engineered bacterium comprises a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and further comprises a deletion of gpsA gene, and optionally further comprises a deletion of mqo gene.

Thus, in even further preferred embodiments the genetically engineered bacterium comprises a deletion of ndh gene, a deletion of one or more nuo genes, a deletion of did gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and further comprises a deletion of one or more genes selected from the group comprising or consisting of: mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

Thus, in even further preferred embodiments the genetically engineered bacterium comprises a deletion of ndh gene, a deletion of one or more nuo genes, a deletion of did gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and further comprises a deletion of gpsA gene, and further comprises a deletion of one or more genes selected from the group comprising or consisting of: mqo gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

Thus, in even further preferred embodiments the genetically engineered bacterium comprises a deletion of ndh gene, a deletion of one or more nuo genes, a deletion of did gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and further comprises a deletion of gpsA gene, and further comprises a deletion of mqo gene, and further comprises a deletion of one or more genes selected from the group comprising or consisting of: poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

In particular preferred embodiments, the genetically engineered bacterium comprises
a deletion of ndh gene;
a deletion of one or more nuo genes;
a deletion of dld gene, and
   a deletion of:
   mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoE gene, nuoF gene, and/or nuoG gene, more preferably nuoEFG genes;
   wherein the genetically engineered bacterium is able of oxygen uptake, i.e. the genetically engineered bacterium is able to use oxygen as electron acceptor, and
   wherein the genetically engineered bacterium is genetically engineered to produce a fermentation product in presence of a terminal electron acceptor, preferably in presence of oxygen. Thus, the genetically engineered bacterium produces a fermentation product from a carbon source in presence of a terminal electron acceptor, preferably in presence of oxygen.

In preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In particularly preferred embodiments, the genetically engineered bacterium is an E. coli. In preferred embodiments, the E. coli is derived from the well-known E. coli K-12 MG1655 strain. In even more preferred embodiments, the E. coli is an E. coli K-12 MG1655 further comprising one or more recombination systems. The one or more recombination systems are preferably selected from an arabinose inducible lambda Red recombineering system and a rhamnose inducible flippase recombinase.

In contrary to the prior art, the genetically engineered bacteria of the present invention have not been modified to completely eliminate the aerobic respiratory pathway. The aerobic respiratory chain involves ubiquinone (Q) mediated electron transfer to cytochrome oxidases. The cytochrome oxidases bo₃, bd-1, bd-II, are the major terminal oxidases in the aerobic respiratory chain and oxidize reduced ubiquinone-8 (UQ-8) and reduce O₂ to two H₂O. Engineered E. coli strains having a deletion of the cyoABCD genes, cydAB genes and appBC genes have been described in US patent application US 2012/0064581 A1. The deletions of these genes prevent the oxygen uptake so that the anaerobic pathways responsible for mixed-acid fermentation are active under aerobic conditions. However, the genetically engineered bacteria of the present invention have not been modified to exclude the possibility of oxygen uptake, which allows alternative fermentation pathways that make the use of alternative carbon sources such as glycerol for the production of substances such as D-lactate, isobutanol and ethanol by fermentation under aerobic conditions possible in the first place. Thus, the genetically engineered bacteria disclosed herein do not comprise a deletion of cyoABCD genes, cydAB genes and/or cbdAD genes. With other words, the genetically engineered bacteria disclosed herein express CyoABCD, CydAB, and CbdAD.

The present invention preferably relates to a genetically engineered E. coli comprising a deletion of ndh gene, nuoEFG genes, and dld gene (NNminimal1).

The present invention preferably further relates to a genetically engineered E. coli comprising a deletion of ndh gene, nuoEFG genes, dld gene, and further comprising a deletion of mqo gene (NNminimal2).

Moreover, the present invention preferably further relates to a genetically engineered E. coli comprising a deletion of ndh gene, nuoEFG genes, dld gene, and further comprising a deletion of mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene (NNmini+glpD).

The genetically engineered bacteria as described above are genetically engineered to produce D-lactate from several carbon sources including, but not limited to glucose, fructose, xylose, arabinose, galactose, mannose, sucrose, cellobiose, hemicellulose, and glycerol or a combination thereof, in presence of oxygen. Thus, the genetically engineered bacteria as described above produce D-lactate from a carbon source including, but not limited to glucose, fructose, xylose, arabinose, galactose, mannose, sucrose, cellobiose, hemicellulose, and glycerol or a combination thereof, in presence of oxygen. Preferably, the carbon source may be glucose or glycerol or a combination thereof. Preferably, the carbon source may be glycerol.

In preferred embodiment, the genetically engineered bacteria as described above do not comprise a deletion of glpD gene, which allows advantageously the use of glycerol as carbon source for the fermentative production of D-lactate. Preferably, the genetically engineered bacteria as described above do not comprise a deletion of glpD gene, but comprise a deletion of gpsA gene.

Thus, the present invention preferably relates to a genetically engineered E. coli comprising a deletion of ndh gene, nuoEFG genes, and dld gene (NNminimal1), wherein the genetically engineered E. coli does not comprise a deletion of glpD gene. With other words, the genetically engineered E. coli expresses GlpD.

Thus, the present invention preferably relates to a genetically engineered E. coli comprising a deletion of ndh gene, nuoEFG genes, dld gene, and further comprising a deletion of mqo gene (NNminimal2), wherein the genetically engineered E. coli does not comprise a deletion of glpD gene. With other words, the genetically engineered E. coli expresses GlpD.

Thus, the present invention particularly preferably relates to a genetically engineered E. coli comprising a deletion of ndh gene, nuoEFG genes, dld gene, and further comprising a deletion of mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene (NNmini+glpD), wherein the genetically engineered E. coli does not comprise a deletion of glpD gene. With other words, the genetically engineered E. coli expresses GlpD.

A further preferred embodiment of the present invention relates to a genetically engineered bacterium comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and further comprising a deletion of ldhA gene, and

wherein the genetically engineered bacterium further comprises an engineered metabolic pathway for converting pyruvate to isobutanol, wherein said engineered metabolic pathway comprises AlsS, IlvC, IlvD, KivD and SdhA. This genetically engineered bacterium is able to produce isobutanol from several carbon sources including, but not limited to glucose, fructose, xylose, arabinose, galactose, mannose, sucrose, cellobiose, hemicellulose, and glycerol or a combination thereof, in presence of a terminal electron acceptor, preferably in presence of oxygen. Particularly preferably, the genetically engineered bacterium for fermentative production of isobutanol and/or ethanol as described above does not comprise a deletion of glpD gene, which allows advantageously the use of glycerol as carbon source for the fermentative production of isobutanol.

In other embodiments of the present invention, the genetically engineered bacteria as described above for the production of D-lactate, isobutanol and/or ethanol, may further comprise a deletion of glpD gene. In such preferred embodiments, the carbon source for the fermentative production of D-lactate, isobutanol and/or ethanol, preferably D-lactate or isobutanol, may be selected from the group comprising or consisting of glucose, fructose, xylose, arabinose, galactose, mannose, sucrose, cellobiose, and hemicellulose, or a combination thereof, more preferably glucose.

A further aspect of the present invention relates to a method for obtaining a fermentation product comprising the following steps:
a) providing a genetically engineered bacterium comprising a deletion of ndh gene; a deletion of one or more nuo genes; a deletion of dld gene, and optionally further comprising a deletion of one or more genes selected from the group comprising or consisting of mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, hemicellulose, and glycerol or a combination thereof;
wherein the supplement is selected from acetate, pyruvate, and casamino acids (CAA).

A further aspect of the present invention relates to a method for obtaining a fermentation product comprising the following steps:
a) providing a genetically engineered bacterium comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and further comprising a deletion of ldhA gene, and wherein the genetically engineered bacterium further comprises an engineered metabolic pathway for converting pyruvate to isobutanol, wherein said engineered metabolic pathway comprises AlsS, IlvC, IlvD, KivD and SdhA;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, hemicellulose, and glycerol or a combination thereof;
wherein the supplement is selected from acetate, pyruvate, and casamino acids (CAA).

A further aspect of the present invention relates to a method for obtaining a fermentation product comprising the following steps:
a) providing a genetically engineered bacterium comprising a deletion of ndh gene; a deletion of one or more nuo genes; a deletion of dld gene, and optionally further comprising a deletion of one or more genes selected from the group comprising or consisting of mqo gene, gplD gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, and hemicellulose, or a combination thereof;
wherein the supplement is selected from acetate, pyruvate, and casamino acids (CAA).

A further aspect of the present invention relates to a method for obtaining a fermentation product comprising the following steps:
a) providing a genetically engineered bacterium comprising a deletion of ndh gene, nuoEFG genes, dld gene, mqo gene, glpD gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and further comprising a deletion of ldhA gene, and wherein the genetically engineered bacterium further comprises an engineered metabolic pathway for converting pyruvate to isobutanol, wherein said engineered metabolic pathway comprises AlsS, IlvC, IlvD, KivD and SdhA;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, and hemicellulose, and or a combination thereof;
wherein the supplement is selected from acetate, pyruvate, and casamino acids (CAA).

A particular advantage of the genetically engineered bacteria of the present invention is that they can be easily reproduced by a skilled person by means known in the art and adaptive evolution is not required to obtain the bacteria of the present invention.

### DESCRIPTION OF THE INVENTION

It has been surprisingly found that a genetically engineered bacterium comprising a deletion of ndh gene, one or more nuo genes, dld gene and optionally mqo gene enables fermentation processes that can take place in spite of the presence of oxygen, i.e. under aerobic conditions, further allowing the use of alternative carbon sources such as glycerol for the production of specific fermentation products, in particular D-lactate.

Moreover, it has been found that a genetically engineered bacterium comprising a deletion of ndh gene, one or more nuo genes, and dld gene, and further comprising a deletion of mqo gene and a deletion of one or more genes for NADPH dehydrogenase activity, such as kefF, wrbA, yieF, mdaB-ygiN genes, reactions of NADH dehydrogenase bypassing activities: putA, glcDEF, dadA, fadE, glpD, lldD, glpABC, lhgO, gpsA, and quinone reducing reactions from central metabolism: poxB, sdhABCD, improved selective production of specific fermentation end products, e.g. in particular D-lactate, and is stable and robust against undesired evolutionary adaptations and mutations.

Therefore, the target genes are grouped on the basis of the activity of encoded proteins as follows:
Group A: NADH-dehydrogenase(s): ndh, nuoEFG;
Group B: NADPH dehydrogenase activity described: kefF, wrbA, yieF, mdaB, ygiN;
Group C: quinone reducing reactions from central metabolism: poxB, sdhABCD;
Group D: reactions of NADH dehydrogenase bypassing activities: dld, mqo, putA, glcDEF, dadA, fadE, glpD, gpsA, lldD, glpABC, lhgO.

The following enzymes are able to form “mini cycles”:
dld, mqo, glpABC, glpD, putA, glcDEF, dadA, fadE, lhgO, gpsA.

The genetically engineered bacteria of the present invention have not been modified to exclude the possibility of oxygen uptake, which allows alternative fermentation pathways that make use of alternative carbon sources such as glycerol for the production of D-lactate by fermentation under aerobic conditions.

Therefore, the present invention relates to a genetically engineered bacterium comprising:
a deletion of ndh gene,
a deletion of one or more nuo genes, and
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor,
   wherein the bacterium is genetically engineered to produce a fermentation product in presence of a terminal electron acceptor, preferably in presence of oxygen. Preferably, the genetically engineered bacterium produces a fermentation product from a carbon source in presence of oxygen. More preferably, the genetically engineered bacterium produces D-lactate from a carbon source in presence of oxygen, wherein the carbon source is preferably selected from the group comprising or consisting of glucose, fructose, xylose, arabinose, galactose, mannose, sucrose, cellobiose, hemicellulose, and glycerol, or a combination thereof.

Thus, the genetically engineered bacteria disclosed herein do not comprise a deletion of cyoABCD genes, cydAB genes and/or cbdAD genes. With other words, the genetically engineered bacteria disclosed herein express CyoABCD, CydAB, and CbdAD.

Preferably, the present invention relates to a genetically engineered bacterium comprising:
a deletion of ndh gene,
a deletion of one or more nuo genes, and
a deletion of dld gene,
   a deletion of one or more genes selected from the group comprising: mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor,
   wherein the bacterium is genetically engineered to produce a fermentation product in presence of a terminal electron acceptor, preferably in presence of oxygen. Preferably, the genetically engineered bacterium produces a fermentation product from a carbon source in presence of oxygen. More preferably, the genetically engineered bacterium produces D-lactate from a carbon source in presence of oxygen, wherein the carbon source is preferably selected from the group comprising or consisting of glucose, fructose, xylose, arabinose, galactose, mannose, sucrose, cellobiose, hemicellulose, and glycerol, or a combination thereof.

In other embodiments, the present invention relates to a genetically engineered bacterium comprising:
a deletion of ndh gene,
a deletion of one or more nuo genes, and
a deletion of dld gene,
   a deletion of one or more genes selected from the group comprising: mqo gene, glpD gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor,
   wherein the bacterium is genetically engineered to produce a fermentation product in presence of a terminal electron acceptor, preferably in presence of oxygen. Preferably, the genetically engineered bacterium produces a fermentation product from a carbon source in presence of oxygen. More preferably, the genetically engineered bacterium produces D-lactate from a carbon source in presence of oxygen, wherein the carbon source is preferably selected from the group comprising or consisting of glucose, fructose, xylose, arabinose, galactose, mannose, sucrose, cellobiose, and hemicellulose, or a combination thereof.

In particular, the present invention relates to a genetically engineered bacterium comprising:
a deletion of ndh gene;
a deletion of one or more nuo genes;
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor,
   wherein the bacterium is genetically engineered to produce a fermentation product in presence of a terminal electron acceptor, preferably in presence of oxygen. Preferably, the genetically engineered bacterium produces a fermentation product from a carbon source in presence of oxygen; and
   wherein the genetically engineered bacterium does not comprise a deletion of cyoABCD genes, cydAB genes and/or cbdAD genes. With other words, the genetically engineered bacterium expresses CyoABCD, CydAB, and CbdAD.

The term “a deletion of one or more genes” is used herein with the same meaning as “one or more gene deletions” and “one or more deletions of a gene”.

Preferred Gene Deletions for the Genetically Engineered E. coli strain

The term “NADH dehydrogenase”, as used herein, relates to an enzyme of EC class 1.6.5.11.that catalyzes the chemical reaction

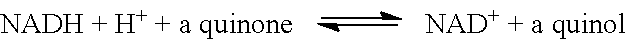

The term “nuo gene”, as used herein, relates to a gene that encodes one subunit of the protein complex NADH:ubiquinone oxidoreductase I (NuoABCDEFGHIJKLMN, NDH-1). The nuo genes in E. coli include 14 genes: nuoABCDEFGHIJKLMN genes, encoding 13-14 subunits, nuoC and nuoD are thought to form one subunit. Thus, NADH:ubiquinone oxidoreductase I of E. coli is made up of 13-14 different subunits, the NuoA to NuoN subunits. This organization in 13-14 different subunits is conserved in several other bacteria, including Salmonella typhimurium, Paracoccus denitrificans, Rhodobacter capsulatus, and Thermus thermophilus. The nuoA gene encodes the subunit NuoA of NDH-1. The nuoB gene encodes the subunit NuoB of NDH-1. The nuoC gene encodes the subunit NuoC of NDH-1 and the nuoD gene encodes the subunit NuoD of NDH-1, or nuoC and nuoD genes encode the nuoCD subunit. The nuoE gene encodes the subunit NuoE of NDH-1. The nuoF gene encodes the subunit NuoF of NDH-1. The nuoG gene encodes the subunit NuoG of NDH-1. The nuoH gene encodes the subunit NuoH of NDH-1. The nuoI gene encodes the subunit NuoI of NDH-1. The nuoJ gene encodes the subunit NuoJ of NDH-1. The nuoK gene encodes the subunit NuoK of NDH-1. The nuoL gene encodes the subunit NuoL of NDH-1. The nuoM gene encodes the subunit NuoM of NDH-1. The nuoN gene encodes the subunit NuoN of NDH-1.NADH:ubiquinone oxidoreductase I (NDH-1) of E. coli consists of three components: a soluble fragment composed of the NuoE, F and G subunits, an amphipathic connecting fragment composed of the NuoB, CD and I subunits, and a hydrophobic membrane fragment composed of the NuoA, H, J, K, L, M and N subunits. Thereby, the soluble NADH:ubiquinone oxidoreductase I fragment consisting of the NuoE, NuoF and NuoG subunits represents the electron input part of NADH:ubiquinone oxidoreductase 1.

The term “NADH:ubiquinone oxidoreductase I” (NDH-1), as used herein, relates to a protein complex of the respiratory chains of many organisms from bacteria to humans. NADH:ubiquinone oxidoreductase I belongs to EC class 7.1.1.2. In E. coli, NADH:ubiquinone oxidoreductase I (NDH-1) is one of two distinct NADH dehydrogenases that catalyze the transfer of electrons from NADH to the quinone pool in the cytoplasmic membrane and is able to generate a proton electrochemical gradient.

The term “ndh gene”, as used herein, relates to a gene that encodes the enzyme NADH:quinone oxidoreductase 1l.

The term “NADH:quinone oxidoreductase II” (NDH-2), as used herein, relates to an alternative, non-proton pumping NADH:quinone oxidoreductase that delivers electrons to the respiratory chain by oxidation of NADH and reduction of quinones. Thus, NADH:quinone oxidoreductase II is one of two distinct NADH dehydrogenases that catalyze the transfer of electrons from NADH to the quinone pool in the cytoplasmic membrane but does not generate an electrochemical gradient as NDH-1 does. NADH:quinone oxidoreductase II. NADH:ubiquinone oxidoreductase II belongs to EC class 1.6.5.9.

The term “KefF”, (synonym: yabF), as used herein, relates to the Glutathione-regulated potassium-efflux system ancillary protein. KefF is an activator of potassium transport mediated by the KefC antiporter. KefF also has enzymatic activity as a quinone oxidoreductase, thereby reducing the redox toxicity of electrophilic quinones.

The term “wrbA gene”, as used herein, relates to a gene that encodes a NAD(P)H dehydrogenase (quinone).

The term “WrbA”, as used herein, relates to a protein that has NAD(P)H:quinone oxidoreductase activity. WrbA is related to the flavodoxin family of proteins. Unlike the flavodoxins, WrbA does not have a stabilized semiquinone state. It rapidly takes up two electrons, generating the fully reduced form.

The term “yieF gene” (synonym: chrR), as used herein, relates to a gene that encodes a quinone reductase.

The term “YieF”, as used herein, relates to a flavoprotein containing the FMN cofactor that belongs to the flavodoxin superfamily of enzymes. YieF was shown to possess quinone reductase activity which may guard against oxidative stress by preventing redox cycling of quinones which would otherwise generate ROS, and by maintaining a pool of reduced quinone in the cell that is able to quench ROS directly. The quinone reductase activity of YieF is considered as the primary biological role of this enzyme.

The term “ygiN gene”, as used herein, relates to a gene in E. coli that encodes the probable quinol monooxygenase YgiN. The ygiN gene may be transcribed in an operon together with mdaB, indicated as mdaB-ygiN.

The term “YgiN”, as used herein, relates to a protein “probable quinol monooxygenase” that is able to re-oxidize menadiol that has been reduced by “MdaB quinone reductase” in vitro. The two enzymes “probable quinol monooxygenase” and “MdaB quinone reductase” may form a quinone redox cycle. The biological role of a quinone redox cycle is considered to maintain an intracellular pool of menadione and ubiquinone using a catalytic mechanism that avoids the formation of a semiquinone intermediate, and to act as a quinone buffer.

The term “mdaB gene”, as used herein, relates to a gene that encodes the NADPH:quinone oxidoreductase MdaB.

The term “MdaB”, as used herein, relates to the protein “MdaB quinone reductase” that is specific for NADPH and is most active with quinone derivatives and ferricyanide as electron acceptors. In vitro, YgiN is able to reoxidize menadiol that has been reduced by MdaB quinone reductase; the two enzymes may form a quinone redox cycle.

The term “mdaB-ygiN genes” or “mdaB-ygiN operon”, as used herein, relates to the genes or operon encoding a NADPH:quinone oxidoreductase and a “probable quinol monooxygenase” presumably forming a quinone redox cycle.

The term “putA gene” (synonym: poaA), as used herein, relates to a gene in that encodes the bifunctional protein PutA.

The term “PutA”, as used herein, relates to the bifunctional protein PutA that is involved in step 1 and 2 of the sub-pathway that synthesizes L-glutamate from L-proline. The bifunctional protein PutA includes the domains proline dehydrogenase and pyrroline-5-carboxylate dehydrogenase. PutA is a flavoprotein with mutually exclusive functions as a transcriptional repressor and membrane-associated enzyme. The switch between the two activities is due to conformational changes triggered by the redox state of FAD. In the presence of L-proline, PutA is associated with the cytoplasmic membrane and acts a bifunctional enzyme catalyzing both reactions of the proline degradation pathway: the oxidation of proline by proline dehydrogenase and subsequent oxidation to glutamate by pyrroline-5-carboxylate (P5C) dehydrogenase. The kinetics of the coupled reaction is best described by substrate channeling. In the absence of proline, PutA is cytoplasmic and functions as a transcriptional repressor of the put regulon. Proline dehydrogenase activity requires the presence of an electron acceptor. The reaction is split into a reductive half reaction, the reduction of the FAD cofactor by oxidation of proline, and an oxidative half reaction, the re-oxidation of reduced FADH2 by transfer of electrons to the quinone pool in the cytoplasmic membrane.

The term “glcDEF genes” (synonyms: gox, yghM), as used herein, relates to the genes glcD, glE, and glcF that encode the subunits of the enzyme glycolate oxidase.

More in particular, the term “GlcDEF”, as used herein, relates to the three subunits of the enzyme glycolate oxidase GlcD, GlcE and GlcF. GlcDEF is a component of a complex that catalyzes the oxidation of glycolate to glyoxylate. GlcDEF is required by bacteria to grow on glycolate as a sole carbon source. The ability to oxidize D-lactate has been described in connection with GlcDEF. GlcDEF does not link directly to O₂.

The term “dld gene”, as used herein, relates to a gene that encodes the enzyme quinone-dependent D-lactate dehydrogenase.

The term “Dld”, as used herein, relates to the enzyme quinone-dependent D-lactate dehydrogenase that is a FAD-dependent peripheral membrane dehydrogenase catalyzing the oxidation of D-lactate to pyruvate. D-lactate dehydrogenase (Dld) is a respiratory enzyme; electrons derived from D-lactate oxidation are transferred to the membrane soluble quinone pool.

The term “mqo gene” (synonym: yojH), as used herein, relates to a gene that encodes the enzyme malate:quinone oxidoreductase (Mqo).

The term “Mqo”, as used herein, relates to the enzyme the malate:quinone oxidoreductase which is a membrane-associated enzyme that catalyzes the oxidation of malate to oxaloacetate. Electrons are likely donated to the electron transfer chain at the quinone level.

The term “dadA gene” (synonym: dadR), as used herein, relates to a gene that encodes the enzyme D-amino acid dehydrogenase (DadA).

The term “DadA”, as used herein, relates to the enzyme D-amino acid dehydrogenase. E L- and D-alanine can be used by bacteria as the sole source of carbon, nitrogen and energy. D-amino acid dehydrogenase is the second enzyme of the L-alanine degradation I pathway. The enzyme has broad substrate specificity; it catalyzes the oxidative deamination of many D-amino acids, although D-alanine is the best substrate. The enzyme is membrane-associated and linked to the respiratory chain.

The term “fadE gene” (synonym: yafH), as used herein, relates to a gene that encodes the enzyme Acyl-coenzyme A dehydrogenase (FadE). fadE mutants are unable to utilize oleate and other fatty acids as the sole source of carbon.

The term “FadE”, as used herein, relates to the enzyme Acyl-coenzyme A dehydrogenase that catalyzes the first step in the degradation of fatty acids via the β-oxidation cycle.

The term “glpD gene” (synonym: yafH), as used herein, relates to a gene that encodes aerobic glycerol-3-phosphate dehydrogenase (GlpD).

The term “GlpD”, as used herein, relates to the enzyme aerobic glycerol-3-phosphate dehydrogenase that catalyzes the oxidation of sn-glycerol 3-phosphate to dihydroxyacetone phosphate. GlpD is a respiratory enzyme and shuttles electrons via a non-covalently bound FAD cofactor to reduce ubiquinone. Glycerol 3-phosphate is an obligatory intermediate in phospholipid biosynthesis and thus glpD expression is regulated to ensure that phospholipid biosynthesis is maintained while the energy needs of the cell are met. GlpD is required for aerobic growth with glycerol or glycerol 3-phosphate.

A glpD deletion is not essential to metabolize a particular carbon source, such as a carbon source selected from the group comprising glucose, fructose, xylose, arabinose, galactose, mannose, sucrose, cellobiose, and hemicellulose or a combination thereof.

However, a glpD deletion seems to induce higher stability in bacteria when growing on a carbon source selected from the group comprising glucose, fructose, xylose, arabinose, galactose, mannose, sucrose, cellobiose, and hemicellulose.

The term “lldD gene” (synonym: lctD), as used herein, relates to a gene that encodes L-lactate dehydrogenase.

The term “LldD”, as used herein, relates to the enzyme L-lactate dehydrogenase that is an FMN-dependent membrane-associated dehydrogenase. It functions in aerobic respiration and also has a role in anaerobic nitrate respiration. L-lactate dehydrogenase is associated with the inner membrane. LldD is one of three lactate dehydrogenase enzymes which interconvert pyruvate and lactate. The other two enzymes are specific for D-lactate: the soluble LdhA, an NAD-linked fermentative enzyme, and Dld, a membrane-associated respiratory enzyme. L-lactate dehydrogenase is induced by aerobic growth on L-lactate and L-fucose and in the presence of lactate under nitrate, fumarate and TMAO respiration conditions.

The term “glpABC genes”, as used herein, relates to the genes that encode anaerobic glycerol-3-phosphate dehydrogenase (GlpABC).

The term “GlpABC”, as used herein, relates to the enzyme anaerobic glycerol-3-phosphate dehydrogenase that catalyzes the oxidation of glycerol-3-phosphate to dihyroxyacetone phosphate. GlpABC is a respiratory enzyme; anaerobic growth of bacteria on glycerol and fumarate induces expression of an anaerobic glycerol-3-phosphate dehydrogenase and fumarate reductase and is associated with proton translocation and the generation of a proton motive force. The GlpABC enzyme is loosely associated with the cell membrane. Bacteria, such as E. coli K-12, contain two glycerol-3-phosphate dehydrogenases encoded by the glpABC and glpD genes. GlpABC is required for anaerobic growth with glycerol or glycerol-3-phosphate and fumarate as the terminal electron acceptor while GlpD is required for aerobic growth with glycerol (or glycerol-3-phosphate).

The term “lhgO gene” (synonym: lhgD), as used herein, relates to a gene that encodes-2-hydroxyglutarate dehydrogenase (LhgO, synonym LhgD).

The term “LhgO”, as used herein, relates to the enzyme L-2-hydroxyglutarate dehydrogenase which is an electron transport chain-coupled dehydrogenase that feeds electrons from the reaction into the membrane quinone pool. LhgD contains an FAD cofactor which is not covalently attached, and whose reduction potential is relatively high at −25 mV. LhgD is associated with the cytoplasmic membrane, and its activity is only found in the membrane fraction.

The term “poxB gene”, as used herein, relates to a gene that encodes pyruvate dehydrogenase [ubiquinone](PoxB).

The term “PoxB”, as used herein, relates to the enzyme pyruvate dehydrogenase [ubiquinone] which is a peripheral membrane enzyme that catalyzes the oxidative decarboxylation of pyruvate to form acetate and CO₂. The reaction is coupled to the electron transport chain via ubiquinone. Metabolism of pyruvate by pyruvate oxidase is less efficient than the route via pyruvate dehydrogenase (PDH); however, the pyruvate oxidase route is important for wild-type growth efficiency and responsible for a significant amount of pyruvate metabolism under aerobic conditions.

The term “sdhABCD genes”, as used herein, relates to the genes encoding succinate dehydrogenase or succinate:quinone oxidoreductase (SdhABCD).

The term “SdhABCD”, as used herein, relates to the enzyme succinate:quinone oxidoreductase that catalyzes the oxidation of succinate to fumarate concomitant with the reduction of ubiquinone to ubiquinol. SdhABCD plays an important role in cellular metabolism and directly connects the TCA cycle with the respiratory electron transport chain. As part of the TCA cycle succinate is oxidized to fumarate by SdhABCD and electrons are transferred to the membrane quinone pool for entry into the electron transport chain. SdhABCD does not contribute to the proton motive force; the sites of quinol reduction and succinate oxidation are both located on the cytoplasmic side of the membrane and there is no separation of charge across the membrane during catalysis. SdhABCD is a membrane bound heterotetramer. Subunits SdhA and SdhB are hydrophilic and attached to the cytoplasmic surface of the plasma membrane via interactions with the two hydrophobic integral membrane subunits, SdhC and SdhD. SdhA contains the FAD cofactor and the dicarboxylic acid binding site. Electrons from the oxidation of succinate are transferred through the iron-sulphur protein, SdhB, to a quinone binding site located at the interface of the SdhB, SdhC and SdhD subunits. The SdhC and SdhD subunits each contain three transmembrane helices and anchor the complex to the membrane. A single heme b556 cofactor bridges the SdhC and SdhD subunits.

The term “gpsA gene”, as used herein, relates to a gene that encodes the enzyme glycerol-3-phosphate dehydrogenase (GpsA).

The term “GpsA”, as used herein, relates to the enzyme glycerol-3-phosphate dehydrogenase catalyzes the NAD(P)H-dependent reduction of the glycolytic intermediate dihydroxyacetone-phosphate to produce glycerol-3-phosphate, a precursor for the biosynthesis of phospholipids.

The term “ldhA gene”, as used herein, relates to a gene that encodes the enzyme D-lactate dehydrogenase (LdhA).

The term “LdhA”, as used herein, relates to the enzyme D-lactate dehydrogenase. LdhA is a soluble NAD-linked lactate dehydrogenase (LDH) that is specific for the production of D-lactate.

The term “ubiC gene” as used herein, relates to a gene that encodes the enzyme chorismate pyruvate-lyase (UbiC).

The term “UbiC”, as used herein, relates to the enzyme chorismate pyruvate-lyase that catalyzes the first reaction step in the biosynthesis of ubiquinone which involves the formation of 4-hydroxybenzoate from chorismate. ubiC mutants are deficient in the formation of ubiquinone and are characterized by the inability to grow aerobically on oxidizable substrates such as succinate.

The term “ubiA gene” as used herein, relates to a gene that encodes the enzyme 4-hydroxybenzoate octaprenyltransferase (UbiA).

The term “UbiA”, as used herein, relates to the enzyme 4-hydroxybenzoate octaprenyltransferase that catalyzes the second reaction step in the biosynthesis of ubiquinone which involves the prenylation of 4-hydroxybenzoate with an all-trans polyprenyl group.

The term “ubiCA genes” or “ubiCA operon” as used herein, relates to the operon encoding the enzymes chorismate lyase and 4-hydroxybenzoate transferase for the first two committed steps of ubiquinone (also named Coenzyme Q, or Coenzyme Q10, UQ) biosynthesis.

The term “metabolic pathway for converting pyruvate to isobutanol” as used herein, relates to the metabolic pathway comprising the enzymes AlsS, IlvC, IvD, KivD and AdhA.

The term “engineered metabolic pathway for converting pyruvate to isobutanol” as used herein, relates to the metabolic pathway comprising the enzymes AlsS, IlvC, IlvD, KivD and AdhA, and wherein the enzymes AlsS, IlvC, IlvD, KivD and AdhA are expressed by genetic engineering.

The term “alsS gene” as used herein, relates to a gene that encodes the enzyme acetolactate synthase (AlsS).

The term “AlsS”, as used herein, relates to the enzyme acetolactate synthase that catalyzes the conversion of two molecules of pyruvate molecules to acetolactate with the release of CO₂, which represents the first reaction step in the conversion of piruvate to isobutanol.

The term “ilvC gene” as used herein, relates to a gene that encodes the enzyme NADH-dependent ketol-acid reductoisomerase (KARI, IlvC from E. coli; EC 1.1.1.86).

The term “IlvC”, as used herein, relates to the enzyme NADH-dependent ketol-acid reductoisomerase that catalyzes the two-step reaction from S-2-acetolactate (S-2-AL) to 2,3-dihydroxy-isovalerate (DHIV), which represents the second reaction step in the conversion of piruvate to isobutanol.

The term “ilvD gene” as used herein, relates to a gene that encodes the enzyme dihydroxy-acid dehydratase (IlvD).

The term “IlvD”, as used herein, relates to the enzyme dihydroxy-acid dehydratase that catalyzes the reaction from 2,3-dihydroxy-isovalerate (DHIV) to ketoisovalerate, which represents the third reaction step in the conversion of piruvate to isobutanol.

The term “kivD gene” as used herein, relates to a gene that encodes the enzyme Ketoisovalerate decarboxylase (Kivd).

The term “KivD”, as used herein, relates to the enzyme Ketoisovalerate decarboxylase that catalyzes the reaction from ketoisovalerate to isobutiraldehyde with the release of CO₂, which represents the fourth reaction step in the conversion of piruvate to isobutanol.

The term “adhA gene” as used herein, relates to a gene that encodes the enzyme Alcohol dehydrogenase (AdhA).

The term “AdhA”, as used herein, relates to the enzyme alcohol dehydrogenase that catalyzes the reaction from isobutiraldehyde to isobutanol with the release of NAD⁺, which represents the fifth and last reaction step in the conversion of piruvate to isobutanol.

For the provision of genetically engineered bacteria enabling fermentation processes that can take place in the presence of oxygen, i.e. under aerobic conditions, and further allowing the use of alternative carbon sources such as glycerol for the production of specific fermentation products, in particular D-lactate, alternative solutions to the complete disruption of the aerobic respiratory chain, e.g. deletion of the a deletion of the cyoABCD genes, cydAB genes and cbdAD genes encoding the three cytochrome oxidases in E. coli cytochrome bo oxidase (CyoABCD), cytochrome bd-I oxidase (CydAB) and cytochrome bd-II oxidase (AppBC) have been envisaged.

The inventors of the present invention suggested that the controlled regulation of oxygen uptake without completely preventing it will have an advantageous effect generating bacteria with a fermentative phenotype which can be used for the production of specific fermentation end products by fermentation under aerobic conditions. Thus, the deletion of the nuo genes and ndh gene were initially targeted in bacteria, as the NADH:ubiquinone oxidoreductase I (NDH-1, NuoABCDEFGHIJKLMN) and NADH:quinone oxidoreductase II (NDH-2) are the two distinct NADH dehydrogenases that catalyze the oxidation of NADH to NAD and mainly catalyze the transfer of electrons from NADH to the quinone pool in the cytoplasmic membrane.

Thus, a genetically engineered bacterium having a deletion of ndh gene and of one or more nuo genes was obtained in the first step. In particular, a deletion of ndh gene and of nuoEFG genes (i.e. nuoE gene, nuoF gene, and nuoG gene) was performed in an E. coli K-12 strain MG1655. The so obtained genetically engineered Δnuo, ΔnuoEFG E. coli strain did not show a reduction in biomass production compared to the wild type. Thus, for provision of an obligate fermenting phenotype of genetically engineered bacteria that allows fermentative process under aerobic condition further manipulations were required.

In the next step, it was considered that the prevention of ubiquinone (Q) mediated electron transfer from the NADH dehydrogenases to cytochrome oxidases by disrupting the synthesis of ubiquinone (Q) itself would lead to the desired obligate fermenting phenotype of genetically engineered bacteria. Thus, in order to prevent ubiquinone (Q) mediated electron transfer from the NADH dehydrogenases to cytochrome oxidases a deletion of ubi genes for prevention of the biosynthesis of ubiquinone was carried out.

Thus, a further genetically engineered bacterium was obtained by deletion of ubiA gene and ubiC gene. A genetically engineered ΔubiCA E. coli strain was thereby obtained. The inventors of the present invention have found that the ΔubiCA E. coli strain mainly produced acetate along D-lactate on glucose or glycerol. This indicates that the ΔubiCA E. coli is still able to get rid of some electrons from NADH to respiration. Thus, for provision of an obligate fermenting E. coli strain further manipulation was necessary.

Thus, a further genetically engineered bacterium was obtained by a deletion of ndh gene, one or more nuo genes, ubiA gene and ubiC gene. A genetically engineered ΔnuoEFG, ΔubiCA E. coli strain having a deletion of ndh gene and nuoEFG genes and a further deletion of ubiCA gene was thereby obtained. In this way indeed a first obligate fermenting E. coli strain could be obtained and was named “NNQ” strain. The inventors of the present invention have found that the nuoEFG, ndh and ubiCA deleted E. coli strain showed strong reduction of production of acetate and selective production of D-lactate on glucose. However, the production of acetate was increased on glycerol in comparison to glucose. Thus, this strain still produced an undesired amount of acetate along D-lactate when cultured on glycerol as carbon source.

Moreover, the genetically engineered Δnuo, ΔnuoEFG, ΔubiCA E. coli strains have shown further disadvantages as a loss of function mutation in ubiE could be identified with the nuo, ndh and ubiCA deletion E. coli strain in further experiments. It is known from the prior art that a mutation in ubiE leads to accumulation of demethylmenaquinone. Therefore, a demethylmenaquinone-dependent NADH-dehydrogenase-like cycle between pyruvate and lactate was observed for this Δnuo, ΔnuoEFG, ΔubiCA E. coli strain strain, with NADH consumption over LdhA and demethylmenaquinone-dependent lactate dehydrogenase over Dld. The demethylmenaquinone is used in the mini-cycle between pyruvate and lactate via the D-lactate dehydrogenase (Dld) and the pyruvate is thereby reduced to lactate by NADH and subsequently re-oxidized to pyruvate, wherein a quinone is reduced and the electrons get into the respiratory chain. The effects of the described loss of function mutation in ubiE correspond to the effect of a further gene deletion of the ubiE gene, resulting both in activation of the mini-cycle between pyruvate and lactate. Thus, a further deletion of the ubiE does not provide a solution to the observed accumulation of demethylmenaquinone following the mutation in ubiE. The acetate production observed in the ΔubiCA E. coli strain and the Δnuo, ΔnuoEFG, ΔubiCA E. coli strain indicates that these E. coli strains may be still capable of electron transfer from NADH into the respiratory chain or may be able to use another electron acceptor instead of ubiquinone e.g. through PoxB activity for acetate production. In consequence, the deletions of one or more ubi genes have been found to produce E. coli strains that are unsteady against undesired evolutionary adaptations and mutations.

It has been described in the prior art that the deletion of the three cytochrome oxidases, i.e. a deletion of the cyoABCD genes, cydAB genes and cbdAD genes and ygiN gene results in utilization of anaerobic respiration pathways even under aerobic growth conditions and in a shift in the quinone pool from ubiquinones to menaquinones. The disruption of the biosynthesis of ubiquinone in E. coli by deletion of one or more ubi genes leads also to a shift in the quinone pool from ubiquinones to menaquinones, as the ubiquinones are not available anymore. However, as described above, it has been observed that in presence of oxygen the E. coli strains having a deletion of one or more ubi genes try to compensate for the lack of ubiquinone in other ways, e.g. evolutionary adaptions and mutations.

Thus, for provision of the desired genetically engineered bacteria a different strategy was necessary. The inventors of the present invention have first identified further suitable enzymes that catalyze quinone-dependent reactions for further gene deletions in Δnuo, ΔnuoEFG E. coli strain. The following genes have been selected for deletions in the Δnuo, ΔnuoEFG bacteria: kefF gene, wrbA gene, yieF gene, mdaB-ygiN genes, genes, putA gene, glcDEF genes, dld gene, mqo gene, dadA gene, fadE gene, lldD gene, glpABC genes, lhgO gene, poxB gene, sdhABCD genes and gpsA gene and/or glpD gene.

It has been found that a genetically engineered bacterium comprising a deletion of ndh gene, one or more nuo genes, and dld gene comprises the minimum required amount of gene deletions to enable fermentation processes that can take place in the presence of oxygen, i.e. under aerobic conditions, allowing the use of alternative carbon sources such as glycerol for the production of specific fermentation products, in particular D-lactate. The genetically engineered bacteria according to the present invention particularly do not have the disadvantages of instability of the genetically engineered bacteria ΔubiCA and Δnuo, ΔnuoEFG, ΔubiCA.

Thus, the present invention relates to a genetically engineered bacterium comprising a deletion of ndh gene, a deletion of one or more nuo genes, and a deletion of dld gene, and optionally a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene. However, genetically engineered bacteria comprising a deletion of ndh gene, a deletion of nuoEFG genes, and a deletion of dld gene, and optionally a deletion of mqo gene, are herein preferred.

Thus, the present invention preferably relates to a genetically engineered bacterium comprising a deletion of ndh gene, a deletion of one or more nuo genes, a deletion of dld gene, and a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene. However, genetically engineered bacteria comprising a deletion of ndh gene, a deletion of nuoEFG genes, a deletion of dld gene, a deletion of mqo gene, are herein preferred.

A stable obligate fermenting bacterium was obtained by deletion of one or more nuo genes, preferably nuoEFG genes, and ndh gene along with one or more of the genes did gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene instead of the ubiCA genes.

These genetically engineered obligate fermenting bacteria are still able to synthesize ubiquinone via the common ubiquinone biosynthesis pathway. The genes kefF, wrbA, yieF, mdaB encode enzymes with NADPH dehydrogenase activity, the genes putA, glcDEF, dld, mqo, dadA, fadE, glpD, gpsA, lldD, glpABC, lhgO encode enzymes with reactions of NADH dehydrogenase bypassing activities and the genes poxB, sdhABCD encode enzymes with quinone reducing reactions from central metabolism.

The term “genetically engineered bacterium”, or “genetically engineered bacteria”, as used herein, relates to a bacterium or bacteria, including a bacterial cell or a bacterial strain that has been subjected to genetic manipulations, herein in particular subjected to gene deletions.

The term “gene deletion”, “deletion of gene” or “deletion mutation” (symbol: delta, A), as used herein, relates in genetics to a mutation in which a part of a sequence of DNA is left out during DNA replication. Any number of nucleotides can be deleted.

In the context of the invention, deletion of a gene may be modification of a gene encoding a desired polypeptide to be produced by the cell and/or a gene encoding a polypeptide involved in production of a primary or secondary metabolite by the cell. The gene can be removed in its entirety, or as an alternative also the deletion of part of the gene might result in a reduction of the activity of the encoded protein.

Deletion of a desired gene in the chromosome can be done with non-homologous as well as with homologous recombination. Homologous recombination is preferred, as it opens the opportunity to introduce, to remove or to simultaneously introduce and remove a functionality. With homologous recombination is intended, the transforming DNA further contains a DNA sequence that is homologous to a genomic target sequence of the specific cell to be engineered. The skilled person will understand that no 100% identity is required to obtain homologous recombination. A percentage identity of 80%, preferably 90%, more preferably 95%, 98% or 99% will also suffice. Generally, the DNA sequence of interest to be inserted in the chromosome by homologous recombination is flanked by homologous sequences with a sufficient length to enable homologous recombination.

For example, a DNA sequence encoding a selection marker, such as kanamicin resistence, is flanked by gene specific upstream and downstream sequences to allow deletion of a target genes and insertion at its place of the selection marker.

An embodiment of the present invention is directed to a genetically engineered E. coli comprising a deletion of ndh gene, nuoEFG genes, dld gene (NNminimal1), wherein the genetically engineered E. coli is able to use oxygen as electron acceptor, wherein the genetically engineered E. coli is genetically engineered to produce a fermentation product in presence of a terminal electron acceptor, preferably in presence of oxygen. Thus, the genetically engineered E. coli produces a fermentation product from a carbon source in presence of oxygen.

Thus, a preferred embodiment of the present invention is directed to a genetically engineered E. coli comprising a deletion of ndh gene, nuoEFG genes, dld gene (NNminimal1), wherein the genetically engineered E. coli is able to use oxygen as electron acceptor, wherein the genetically engineered E. coli produces a fermentation product from a carbon source in presence of oxygen.

A further embodiment of the present invention is directed to a genetically engineered E. coli comprising deletion of ndh gene, nuoEFG genes, dld gene, and further comprising a deletion of mqo gene (NNminimal2), wherein the genetically engineered E. coli is able to use oxygen as electron acceptor, wherein the genetically engineered E. coli is genetically engineered to produce a fermentation product in presence of a terminal electron acceptor, preferably in presence of oxygen. Thus, the genetically engineered E. coli produces a fermentation product from a carbon source in presence of oxygen.

A further preferred embodiment of the present invention is directed to a genetically engineered E. coli comprising deletion of ndh gene, nuoEFG genes, dld gene, and further comprising a deletion of mqo gene (NNminimal2), wherein the genetically engineered E. coli is able to use oxygen as electron acceptor, wherein genetically engineered E. coli is genetically engineered to produce a fermentation product in presence of oxygen.

A further preferred embodiment of the present invention is directed to a genetically engineered E. coli comprising a deletion of ndh gene, nuoEFG genes, dld gene (NNminimal1), and further comprising a deletion of gpsA gene, wherein the genetically engineered E. coli is able to use oxygen as electron acceptor, wherein the genetically engineered E. coli is genetically engineered to produce a fermentation product in presence of oxygen, i.e. produces a fermentation product from a carbon source in presence of oxygen.

A further preferred alternative embodiment of the present invention is directed to a genetically engineered E. coli comprising a deletion of ndh gene, nuoEFG genes, did gene, and further comprising a deletion of mqo gene (NNminimal2), and further comprising a deletion of gpsA gene, wherein the genetically engineered E. coli is able to use oxygen as electron acceptor, wherein the genetically engineered E. coli is genetically engineered to produce a fermentation product in presence of oxygen, i.e. produces a fermentation product from a carbon source in presence of oxygen.

An alternative embodiment of the present invention is directed to a genetically engineered genetically engineered E. coli comprising a deletion of ndh gene, nuoEFG genes, dld gene (NNminimal1), and further comprising a deletion of glpD gene, wherein the genetically engineered E. coli is able to use oxygen as electron acceptor, wherein the genetically engineered E. coli is genetically engineered to produce a fermentation product in presence of oxygen, i.e. produces a fermentation product from a carbon source in presence of oxygen.

A further preferred alternative embodiment of the present invention is directed to a genetically engineered E. coli comprising a deletion of ndh gene, of nuoEFG genes, of dld gene, and further comprising a deletion of mqo gene (NNminimal2), and further comprising a deletion of glpD gene, wherein the genetically engineered bacteria is able to use oxygen as electron acceptor, wherein the genetically engineered E. coli is genetically engineered to produce a fermentation product in presence of oxygen, i.e. produces a fermentation product from a carbon source in presence of oxygen.

### Preferred Embodiments for NNminimal1

The present invention relates to a genetically engineered bacterium comprising:
a deletion of ndh gene;
a deletion of one or more nuo genes;
a deletion of dld gene; and
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacterium is able to use oxygen as electron acceptor, and
   wherein the bacterium is genetically engineered to produce a fermentation product in presence of a terminal electron acceptor, preferably in presence of oxygen. Thus, the genetically engineered E. coli produces a fermentation product from a carbon source in presence of oxygen. Preferably, the one or more nuo genes are nuoE gene, nuoF gene, and nuoG genes, i.e. nuoEFG genes.

In preferred embodiments, the genetically engineered bacterium, preferably a genetically engineered E. coli, may further comprise a deletion of glpABC genes and/or of gpsA gene. Preferably, the present invention preferably relates to a genetically engineered bacterium, preferably a genetically engineered E. coli, comprising a deletion of ndh gene, a deletion of one or more nuo genes, a deletion of dld gene; and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of poxB gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of sdhABCD genes, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of poxB gene, a deletion of sdhABCD genes, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of poxB gene, and further comprising a deletion of lldD gene and/or dadA gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of sdhABCD genes, and further comprising a deletion of lldD gene and/or of dadA gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of poxB gene, a deletion of sdhABCD genes, and further comprising a deletion of lldD gene and/or of dadA gene, and further comprising a deletion of kefF gene and/or of mdaB and/or ygiN gene, and further comprising a deletion of glpABC genes and/or of gpsA gene. Preferably, the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene. Even more preferably, the one or more nuo genes are nuoE gene, nuoF gene, and nuoG genes, i.e. nuoEFG genes.

Thus, in a preferred embodiment, the present invention relates to a genetically engineered bacterium, preferably a genetically engineered E. coli, comprising a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, and further comprising a deletion of one or more genes selected from the group comprising kefF gene, mdaB-ygiN genes, glpABC genes, gpsA gene, wrbA gene, yieF gene, and fadE gene.

An embodiment of the invention is also directed to a genetically engineered bacterium, preferably a genetically engineered E. coli, comprising a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, and further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A further more preferred embodiment of the invention is directed to a genetically engineered bacterium, preferably a genetically engineered E. coli, comprising a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, and a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of one or more nuo genes, a deletion of dld gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A more preferred embodiment is directed to a genetically engineered bacterium, preferably a genetically engineered E. coli, comprising a deletion of ndh gene, a deletion of one or more nuo genes, a deletion of dld gene, a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, and dld gene, and further comprising a deletion of one or more genes selected from the group comprising: mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, and dld gene, and further comprising a deletion of one or more genes selected from the group comprising: mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from the group comprising or consisting of glucose, fructose, xylose, arabinose, galactose, mannose, sucrose, cellobiose, hemicellulose, and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, and dld gene, and further comprising a deletion of one or more genes selected from the group comprising: mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from glucose and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, and dld gene, and further comprising a deletion of one or more genes selected from the group comprising: mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glucose.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, and dld gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glycerol.

### Preferred Embodiments for NNminimal2

The present invention further relates to a genetically engineered bacterium comprising:
a deletion of ndh gene;
a deletion of one or more nuo genes;
a deletion of dld gene;
a deletion of mqo gene; and
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacterium is able of oxygen uptake, i.e. the genetically engineered bacteria is able to use oxygen as electron acceptor, and
   wherein the bacterium is genetically engineered to produce a fermentation product in presence of a terminal electron acceptor, preferably in presence of oxygen. Thus, the genetically engineered E. coli produces a fermentation product from a carbon source in presence of oxygen. Preferably, the one or more nuo genes are nuoE gene, nuoF gene, and nuoG gene, i.e. nuoEFG genes.

Preferably, the present invention relates to a genetically engineered bacterium, preferably a genetically engineered E. coli, comprising a deletion of ndh gene, a deletion of one or more nuo genes, a deletion of dld gene, a deletion of mqo gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of mqo gene, a deletion of sdhABCD genes, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of lhgO gene, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of lhgO gene, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of putA gene, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of lhgO gene, a deletion of putA gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, and further comprising a deletion of lldD gene and/or of dadA gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene, a deletion of mqo gene, a deletion of sdhABCD genes, and further comprising a deletion of lldD gene and/or of dadA gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, and further comprising a deletion of lldD gene and/or of dadA gene, and further comprising a deletion of kefF gene and/or of mdaB gene and/or of ygiN gene, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of putA gene, and further comprising a deletion of lldD gene and/or of dadA gene, and further comprising a deletion of kefF gene and/or of mdaB-ygiN genes, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, and further comprising a deletion of lldD gene and/or of dadA gene, and further comprising a deletion of kefF gene and/or of mdaB-ygiN genes, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or gpsA gene. Moreover, the present invention relates to a genetically engineered bacterium, preferably a genetically engineered E. coli, comprising a deletion of ndh gene, a deletion of one or more nuo genes, a deletion of did gene; a deletion of mqo gene, a deletion of poxB gene, a deletion of sdhABCD genes, a deletion of glcDEF genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene. Even more preferably, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of putA gene, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of dld gene; a deletion of mqo gene, a deletion of poxB gene, a deletion of sdhABCD genes, a deletion of glcDEF genes, a deletion of putA gene, and further comprising a deletion of lldD gene and/or of dadA gene and further comprising a deletion of kefF gene and/or of mdaB-ygiN genes, and further comprising a deletion of glpABC genes and/or gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene, and further comprising a deletion of wrbA gene and/or of yieF gene. Moreover, the present invention relates to a genetically engineered bacterium, preferably a genetically engineered E. coli, comprising a deletion of ndh gene, a deletion of one or more nuo genes, a deletion of did gene, a deletion of mqo gene, a deletion of poxB gene, a deletion of sdhABCD genes, a deletion of glcDEF genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene, and further comprising a deletion of wrbA gene and/or of yieF gene. Even more preferably, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of putA gene, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of did gene, a deletion of mqo gene, a deletion of poxB gene, a deletion of sdhABCD genes, a deletion of glcDEF genes, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of did gene, a deletion of mqo gene, a deletion of poxB gene, a deletion of sdhABCD genes, a deletion of glcDEF genes, a deletion of lhgO gene, and further comprising a deletion of lldD gene and/or of dadA gene, and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of glcDEF genes, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Moreover, the present invention relates to a genetically engineered bacterium, preferably a genetically engineered E. coli, comprising a deletion of ndh gene, a deletion of one or more nuo genes, a deletion of dld gene, a deletion of mqo gene, a deletion of poxB gene, a deletion of sdhABCD genes, a deletion of glcDEF genes, a deletion of lhgO gene, a deletion of putA gene, and further comprising a deletion of lldD gene and/or of dadA gene and further comprising a deletion of kefF gene and/or of mdaB-ygiN genes, and further comprising a deletion of glpABC genes and/or of gpsA gene, and further comprising a deletion of wrbA gene and/or of yieF gene, and further comprising a deletion of fadE gene. Preferably, the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene. Even more preferably, the one or more nuo genes are nuoE gene, nuoF gene, and nuoG genes, i.e. nuoEFG genes.

The present invention preferably refers to a genetically engineered bacterium, preferably a genetically engineered E. coli, comprising a deletion of ndh gene, a deletion of one or more nuo genes, a deletion of dld gene, a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of one or more genes selected from the group comprising kefF gene, mdaB-ygiN genes, glpABC genes, gpsA gene, wrbA gene, yieF gene, and fadE gene.

A preferred embodiment of the invention is directed to a genetically engineered bacterium, preferably a genetically engineered E. coli, comprising a deletion of ndh gene, a deletion of one or more nuo genes, a deletion of dld gene, a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A still preferred embodiment of the invention is directed to a genetically engineered bacterium, preferably a genetically engineered E. coli, comprising a deletion of ndh gene, a deletion of one or more nuo genes, a deletion of dld gene, a deletion of mqo gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and mqo gene, and further comprising a deletion of one or more genes selected from the group comprising: gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and mqo gene, and further comprising a deletion of one or more genes selected from the group comprising: gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from the group comprising or consisting of glucose, fructose, xylose, arabinose, galactose, mannose, sucrose, cellobiose, hemicellulose, and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and mqo gene, and further comprising a deletion of one or more genes selected from the group comprising: gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from glucose and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and mqo gene, and further comprising a deletion of one or more genes selected from the group comprising: gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glucose.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and mqo gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glycerol.

### Preferred Embodiment for NNmini Strain +glpD

Preferably, the present invention relates to a genetically engineered bacterium comprising a deletion of ndh gene, a deletion of one or more nuo genes, a deletion of dld gene; and further comprising a deletion of gpsA gene. The one or more nuo genes may be selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoNgene.

Thus, the present invention preferably relates to a genetically engineered bacterium comprising:
a deletion of ndh gene;
a deletion of one or more nuo genes;
a deletion of dld gene; and
a deletion of gpsA gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able of oxygen uptake, i.e. the genetically engineered bacteria is able to use oxygen as electron acceptor, and
   wherein the bacteria is genetically engineered to produce a fermentation product in presence of a terminal electron acceptor, preferably in presence of oxygen. Preferably, the genetically engineered bacterium does not comprise a deletion of glpD gene in case gpsA gene is already deleted. Thus, the genetically engineered E. coli produces a fermentation product from a carbon source in presence of oxygen. Preferably, the one or more nuo genes are nuoE gene, nuoF gene, and nuoG genes, i.e. nuoEFG genes.

Thus, the present invention preferably relates to a genetically engineered bacterium comprising:
a deletion of ndh gene;
a deletion of one or more nuo genes;
a deletion of dld gene; and
a deletion of gpsA gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able of oxygen uptake, i.e. the genetically engineered bacteria is able to use oxygen as electron acceptor,
   wherein the bacteria is genetically engineered to produce a fermentation product in presence of a terminal electron acceptor, preferably in presence of oxygen, and wherein the genetically engineered bacterium does not comprise a deletion of glpD gene.

Thus, the genetically engineered E. coli produces a fermentation product from a carbon source in presence of oxygen. Preferably, the one or more nuo genes are nuoE gene, nuoF gene, and nuoG genes, i.e. nuoEFG genes.

Preferably, the present invention relates to a genetically engineered bacterium comprising a deletion of ndh gene, a deletion of one or more nuo genes, a deletion of dld gene; and further comprising a deletion of gpsA gene. The one or more nuo genes may be selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoNgene.

Thus, the present invention preferably relates to a genetically engineered bacterium comprising:
a deletion of ndh gene;
a deletion of one or more nuo genes;
a deletion of dld gene;
a deletion of mqo gene; and
a deletion of gpsA gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacteria is able of oxygen uptake, i.e. the genetically engineered bacteria is able to use oxygen as electron acceptor, and
   wherein the bacteria is genetically engineered to produce a fermentation product in presence of a terminal electron acceptor, preferably in presence of oxygen. Preferably, the genetically engineered bacterium does not comprise a deletion of glpD gene in case gpsA gene is already deleted. Thus, the genetically engineered E. coli produces a fermentation product from a carbon source in presence of oxygen. Preferably, the one or more nuo genes are nuoE gene, nuoF gene, and nuoG genes, i.e. nuoEFG genes.

Thus, the present invention preferably relates to a genetically engineered bacterium comprising:
a deletion of ndh gene;
a deletion of one or more nuo genes;
a deletion of dld gene;
a deletion of mqo gene; and
a deletion of gpsA gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacteria is able of oxygen uptake, i.e. the genetically engineered bacteria is able to use oxygen as electron acceptor, and
   wherein the bacteria is genetically engineered to produce a fermentation product in presence of a terminal electron acceptor, preferably in presence of oxygen,
   wherein the genetically engineered bacterium does not comprise a deletion of glpD gene. Thus, the genetically engineered E. coli produces a fermentation product from a carbon source in presence of oxygen. Preferably, the one or more nuo genes are nuoE gene, nuoF gene, and nuoG genes, i.e. nuoEFG genes.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, and gpsA gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, and gpsA gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from the group comprising or consisting of glucose, fructose, xylose, arabinose, galactose, mannose, sucrose, cellobiose, hemicellulose, and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, and gpsA gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from glucose and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, and gpsA gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glucose.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, preferably nuoEFG genes, dld gene, and gpsA gene, and further comprising a deletion of one or more genes selected from the group comprising: mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glycerol.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, mqo gene, and gpsA gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and wherein the genetically engineered bacterium produces D-lactate from a carbon source in presence of a terminal electron acceptor, preferably in presence of oxygen, wherein the genetically engineered bacterium is able to use oxygen as electron acceptor.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, mqo gene, and gpsA gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from the group comprising or consisting of glucose, fructose, xylose, arabinose, galactose, mannose, sucrose, cellobiose, hemicellulose, and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, mqo gene, and gpsA gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from glucose and glycerol or a combination thereof.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, mqo gene, and gpsA gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glucose.

The present invention preferably relates a genetically engineered bacterium, preferably a genetically engineered E. coli, comprising a deletion of ndh gene, a deletion of one or more nuo genes selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes, dld gene, mqo gene, and gpsA gene, and further comprising a deletion of one or more genes selected from the group comprising: poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glycerol.

The present invention preferably relates a genetically engineered bacterium, preferably a genetically engineered E. coli, preferably a genetically engineered E. coli, comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor.

The present invention preferably relates a genetically engineered bacterium, preferably a genetically engineered E. coli, comprising a deletion of ndh gene, nuoEFG genes, did gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from the group comprising or consisting of glucose, fructose, xylose, arabinose, galactose, mannose, sucrose, cellobiose, hemicellulose, and glycerol or a combination thereof.

The present invention preferably relates a genetically engineered bacterium, preferably a genetically engineered E. coli, comprising a deletion of ndh gene, nuoEFG genes, did gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is selected from glucose and glycerol or a combination thereof.

The present invention preferably relates a genetically engineered bacterium, preferably a genetically engineered E. coli, comprising a deletion of ndh gene, nuoEFG genes, did gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glucose.

The present invention preferably relates a genetically engineered bacterium, preferably a genetically engineered E. coli, comprising a deletion of ndh gene, nuoEFG genes, did gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and
wherein the genetically engineered bacterium produces D-lactate from a carbon source in presence of a terminal electron acceptor, preferably in presence of oxygen,
wherein the genetically engineered bacterium is able to use oxygen as electron acceptor,
wherein the carbon source is glycerol.

Thus, another aspect of the present invention is directed to a genetically engineered bacterium, preferably a genetically engineered E. coli, comprising a deletion of ndh gene, of nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and wherein the bacterium produces D-lactate from a carbon source in presence of a terminal electron acceptor, preferably in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen. Preferably, the one or more nuo genes are nuoE gene, nuoF gene, and nuoG genes, i.e. nuoEFG genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able to use oxygen as electron acceptor, the genetically engineered bacterium produces a fermentation product from a carbon source in presence of oxygen. Preferably, the one or more nuo genes are nuoE gene, nuoF gene, and nuoG genes, i.e. nuoEFG genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen, wherein the genetically engineered bacterium further comprises a deletion of mqo gene.

Thus, the genetically engineered bacterium produces a fermentation product from a carbon source in presence of oxygen. Preferably, the one or more nuo genes are nuoE gene, nuoF gene, and nuoG genes, i.e. nuoEFG genes. The genetically engineered E. coli bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of one or more genes selected from the group comprising: poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene. Thus, the genetically engineered bacterium produces a fermentation product from a carbon source in presence of oxygen. Preferably, the one or more nuo genes are nuoE gene, nuoF gene, and nuoG genes, i.e. nuoEFG genes. The genetically engineered E. coli bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of mqo gene and further comprises a deletion of one or more genes selected from the group comprising: poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene. Thus, the genetically engineered bacterium produces a fermentation product from a carbon source in presence of oxygen. Preferably, the one or more nuo genes are nuoE gene, nuoF gene, and nuoG genes, i.e. nuoEFG genes. The genetically engineered E. coli bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of gpsA gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of mqo gene poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene. Thus, the genetically engineered bacterium produces a fermentation product from a carbon source in presence of oxygen. Preferably, the one or more nuo genes are nuoE gene, nuoF gene, and nuoG genes, i.e. nuoEFG genes. The genetically engineered E. coli bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

### Preferred Embodiments for NNmini+gpsA

The present invention also refers to a genetically engineered bacterium, preferably a genetically engineered E. coli, comprising a deletion of ndh gene, a deletion of one or more nuo genes, a deletion of dld gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of one or more genes selected from the group comprising kefF gene, mdaB-ygiN genes, glpABC genes, gpsA gene, wrbA gene, yieF gene, and fadE gene.

A more preferred embodiment of the invention is directed to a genetically engineered bacterium, preferably a genetically engineered E. coli, comprising a deletion of ndh gene, a deletion of one or more nuo genes, a deletion of dld gene, a deletion of mqo gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A still more preferred embodiment of the invention is directed to a genetically engineered bacterium, preferably a genetically engineered E. coli, comprising a deletion of ndh gene, a deletion of one or more nuo genes, a deletion of dld gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A further preferred embodiment of the invention is directed to a genetically engineered bacterium, preferably a genetically engineered E. coli, comprising a deletion of ndh gene, a deletion of one or more nuo genes, a deletion of dld gene, a deletion of mqo gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A still more preferred embodiment of the invention is directed to a genetically engineered bacterium, preferably a genetically engineered E. coli, comprising a deletion of ndh gene, a deletion of one or more nuo genes, a deletion of dld gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A further preferred embodiment is directed to a genetically engineered bacterium, preferably a genetically engineered E. coli, comprising a deletion of ndh gene, a deletion of one or more nuo genes, a deletion of dld gene, a deletion of mqo gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A more preferred embodiment is directed to a genetically engineered bacterium, preferably a genetically engineered E. coli, comprising a deletion of ndh gene, a deletion of one or more nuo genes, a deletion of dld gene, a deletion of glpD gene, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, and further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, a deletion of glpD gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene a deletion of nuoE, a deletion of nuoF, a deletion of nuoG, a deletion of dld gene, a deletion of glpD gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, a deletion of glpD gene, further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of glpD gene, further comprising a deletion of poxB gene and/or a deletion of sdhABCD genes, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, a deletion of glpD gene, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of glpD gene, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

A preferred embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of mqo gene, a deletion of glpD gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

Another embodiment is directed to a genetically engineered bacterium, preferably a genetically engineered E. coli, comprising a deletion of ndh gene, a deletion of nuoE, a deletion of nuoF, a deletion of nuoG, a deletion of dld gene, a deletion of glpD gene, further comprising a deletion of poxB gene and/or a deletion of sdhABCD genes, a deletion of one or more genes selected from the group comprising glcDEF genes, of lhgO gene, of putA gene, of lldD gene and/or of dadA gene, and further comprising a deletion of one or more genes selected from the group comprising kefF gene, mdaB-ygiN genes, glpABC genes, gpsA gene, wrbA gene, yieF gene, and fadE gene.

Another preferred embodiment of the present invention is directed to genetically engineered bacterium, preferably a genetically engineered E. coli, comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene, and
the bacterium produces D-lactate from a carbon source in presence of a terminal electron acceptor, preferably in presence of oxygen,
wherein the carbon source is selected from the group comprising or consisting of glucose, fructose, xylose, arabinose, galactose, mannose, sucrose, cellobiose, and hemicellulose, or a combination thereof,
wherein the genetically engineered bacteria is able to use oxygen as electron acceptor.

In some embodiments, the genetically engineered bacterium further comprises a deletion of gpsA gene. The genetically engineered E. coli bacterium does not comprise a deletion of cyoABCD genes, cydAB genes and/or cbdAD genes.

Another preferred embodiment of the present invention is directed to a genetically engineered bacterium, preferably a genetically engineered E. coli, comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene, and
wherein the bacterium produces D-lactate from a carbon source in presence of a terminal electron acceptor, preferably in presence of oxygen,
wherein the carbon source is glucose,
wherein the genetically engineered bacteria is able to use oxygen as electron acceptor.

The genetically engineered bacterium, preferably a genetically engineered E. coli, does not comprise a deletion of cyoABCD genes, cydAB genes and/or cbdAD genes.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Another further preferred embodiment of the present invention is directed to a genetically engineered E. coli bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene, and
wherein the bacterium produces D-lactate from a carbon source in presence of a terminal electron acceptor, preferably in presence of oxygen,
wherein the carbon source is selected from the group comprising or consisting of glucose, fructose, xylose, arabinose, galactose, mannose, sucrose, cellobiose, and hemicellulose, or a combination thereof,
wherein the genetically engineered bacterium is able to use oxygen as electron acceptor.

In some embodiments, the genetically engineered E. coli bacterium further comprises a deletion of gpsA gene. The genetically engineered E. coli bacterium does not comprise a deletion of cyoABCD genes, cydAB genes and/or cbdAD genes.

Another further preferred embodiment of the present invention is directed to a genetically engineered E. coli bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene, and
wherein the bacterium produces D-lactate from a carbon source in presence of a terminal electron acceptor, preferably in presence of oxygen,
wherein the carbon source is glucose,
wherein the genetically engineered bacterium is able to use oxygen as electron acceptor.

In some embodiments, the genetically engineered E. coli bacterium further comprises a deletion of gpsA gene. The genetically engineered E. coli bacterium does not comprise a deletion of cyoABCD genes, cydAB genes and/or cbdAD genes.

Thus, another further preferred embodiment of the present invention is directed to a genetically engineered E. coli bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene, and
wherein the carbon source is selected from the group comprising or consisting of glucose, fructose, xylose, arabinose, galactose, mannose, sucrose, cellobiose, and hemicellulose, or a combination thereof,
wherein the genetically engineered bacterium is able to use oxygen as electron acceptor.

In some embodiments, the genetically engineered E. coli bacterium further comprises a deletion of gpsA gene. The genetically engineered E. coli bacterium does not comprise a deletion of cyoABCD genes, cydAB genes and/or cbdAD genes.

Thus, the present invention also relates to a genetically engineered bacterium comprising:
a deletion of ndh gene;
a deletion of one or more nuo genes;
a deletion of dld gene,
a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen. Preferably, the one or more nuo genes are nuoE gene, nuoF gene, and nuoG genes, i.e. nuoEFG genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able to use oxygen as electron acceptor, the genetically engineered bacterium produces a fermentation product from a carbon source in presence of oxygen. Preferably, the one or more nuo genes are nuoE gene, nuoF gene, and nuoG genes, i.e. nuoEFG genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene,
a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of mqo gene.

Thus, the genetically engineered bacterium produces a fermentation product from a carbon source in presence of oxygen. Preferably, the one or more nuo genes are nuoE gene, nuoF gene, and nuoG genes, i.e. nuoEFG genes. In some embodiments, the genetically engineered bacterium further comprises a deletion of gpsA gene. The genetically engineered bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene, a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of one or more genes selected from the group comprising:poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

Thus, the genetically engineered bacterium produces a fermentation product from a carbon source in presence of oxygen. Preferably, the one or more nuo genes are nuoE gene, nuoF gene, and nuoG genes, i.e. nuoEFG genes. In some embodiments, the genetically engineered bacterium further comprises a deletion of gpsA gene. The genetically engineered bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene, a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of mqo gene and further comprises a deletion of one or more genes selected from the group comprising: poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

Thus, the genetically engineered bacterium produces a fermentation product from a carbon source in presence of oxygen. Preferably, the one or more nuo genes are nuoE gene, nuoF gene, and nuoG genes, i.e. nuoEFG genes. In some embodiments, the genetically engineered bacterium further comprises a deletion of gpsA gene. The genetically engineered bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Thus, the present invention also relates to a genetically engineered bacterium comprising:
a deletion of ndh gene;
a deletion of one or more nuo genes;
a deletion of dld gene, a deletion of glpD gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably in the presence of oxygen,
   wherein the genetically engineered bacterium further comprises a deletion of mqo gene poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

Thus, the genetically engineered bacterium produces a fermentation product from a carbon source in presence of oxygen. Preferably, the one or more nuo genes are nuoE gene, nuoF gene, and nuoG genes, i.e. nuoEFG genes. In some embodiments, the genetically engineered bacterium further comprises a deletion of gpsA gene. The genetically engineered bacterium does not comprise a deletion of cyoABCD genes, cydAB genes and/or cbdAD genes. In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

### Preferred Bacteria

The genetically engineered bacterium preferably may be Enterobacteriaceae, Staphylococcaceae, Streptococcaceae, Lactobacillaceae, Vibrionaceae, Pasteurellaceae, Pseudomonidae, Bacilli, Corynebacteria, Gluconobacteria, Acetobacteria, Methylobacteria and the like.

Since several bacterial genomes have been sequenced and are online available in different specific databases, as listed at en.wikipedia.org/wiki/List_of_sequenced_bacterial_genomes, a person of ordinary skill in the art can easily confirm homologous gene sets by sequence alignment, and in particular using amino acid sequences, which diverge less than gene sequences in the prokaryotes.

Therefore, a preferred embodiment of the present invention discloses a genetically engineered bacteria comprising the gene deletions disclosed above, wherein the genetically engineered bacteria is a facultative anaerobic bacteria selected from the group comprising Enterobacteriaceae, Staphylococcaceae, Streptococcaceae, Lactobacillaceae, Vibrionaceae, and Pasteurellaceae.

Therefore, the present invention relates to a genetically engineered bacterium comprising:
a deletion of ndh gene, a deletion of one or more nuo genes, and a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of a terminal electron acceptor, preferably wherein the bacterium is genetically engineered to produce a fermentation product in presence of oxygen,
   wherein the genetically engineered bacteria is derived from a facultative anaerobic bacteria selected from the group comprising Enterobacteriaceae, Staphylococcaceae, Streptococcaceae, Lactobacillaceae, Vibrionaceae, and Pasteurellaceae. In preferred embodiments, the genetically engineered bacterium is an E. coli.

Therefore, the present invention preferably relates to a genetically engineered bacterium comprising:
a deletion of ndh gene,
a deletion of one or more nuo genes, and
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacteria is genetically engineered to produce a fermentation product in presence of oxygen,
   wherein the genetically engineered bacteria is derived from a facultative anaerobic bacterium selected from the group comprising Enterobacteriaceae, Staphylococcaceae, Streptococcaceae, Lactobacillaceae, Vibrionaceae, and Pasteurellaceae. In preferred embodiments, the genetically engineered bacterium is an E. coli.

Preferably, the present invention relates to a genetically engineered bacterium comprising:
a deletion of ndh gene,
a deletion of one or more nuo genes, and
a deletion of dld gene,
   a deletion of one or more genes selected from the group comprising: mqo gene, glpD gene, poxB gene, sdhABCD genes, glcDEF genes, lhgO gene, putA gene, lldD gene, dadA gene, kefF gene, mdaB-ygiN genes, glpABC genes, gpsA gene, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, wherein the genetically engineered bacteria is able to use oxygen as electron acceptor,
   wherein the bacterium is genetically engineered to produce a fermentation product in presence of a terminal electron acceptor, preferably wherein the bacterium is genetically engineered to produce a fermentation product in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In preferred embodiments, the genetically engineered bacterium is an E. coli.

Preferably, the present invention thus relates to a genetically engineered bacteria comprising:
a deletion of ndh gene,
a deletion of one or more nuo genes, and
a deletion of dld gene,
   a deletion of one or more genes selected from the group comprising: mqo gene, glpD gene, poxB gene, sdhABCD genes, glcDEF genes, lhgO gene, putA gene, lldD gene, dadA gene, kefF gene, mdaB-ygiN genes, glpABC genes, gpsA gene, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacterium is able to use oxygen as electron acceptor,
   wherein the bacterium is genetically engineered to produce a fermentation product in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In preferred embodiments, the genetically engineered bacterium is an E. coli.

More preferably, the present invention relates to a genetically engineered bacteria comprising:
a deletion of ndh gene,
a deletion of one or more nuo genes, and
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able to use oxygen as electron acceptor,
   wherein the bacterium is genetically engineered to produce a fermentation product in presence of a terminal electron acceptor, preferably wherein the bacterium is genetically engineered to produce a fermentation product in presence of oxygen,
   wherein the genetically engineered bacterium is an E. coli.

More preferably, the present invention relates to a genetically engineered bacteria comprising:
a deletion of ndh gene,
a deletion of one or more nuo genes, and
a deletion of dld gene,
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene, wherein the genetically engineered bacteria is able to use oxygen as electron acceptor,
   wherein the bacteria is genetically engineered to produce a fermentation product in presence of oxygen,
   wherein the genetically engineered bacterium is an E. coli.

Still more preferably, the present invention relates to a genetically engineered bacteria comprising:
a deletion of ndh gene,
a deletion of one or more nuo genes, and
a deletion of dld gene,
   a deletion of one or more genes selected from the group comprising: mqo gene, glpD gene, poxB gene, sdhABCD genes, glcDEF genes, lhgO gene, putA gene, lldD gene, dadA gene, kefF gene, mdaB-ygiN genes, glpABC genes, gpsA gene, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene, and nuoN gene;
   wherein the genetically engineered bacteria is able to use oxygen as electron acceptor,
   wherein the bacterium is genetically engineered to produce a fermentation product in presence of a terminal electron acceptor, preferably wherein the bacterium is genetically engineered to produce a fermentation product in presence of oxygen,
   wherein the genetically engineered bacterium is an E. coli.

Still more preferably, the present invention relates to a genetically engineered bacteria comprising:
a deletion of ndh gene,
a deletion of one or more nuo genes, and
a deletion of dld gene,
   a deletion of one or more genes selected from the group comprising: mqo gene, glpD gene, poxB gene, sdhABCD genes, glcDEF genes, lhgO gene, putA gene, lldD gene, dadA gene, kefF gene, mdaB-ygiN genes, glpABC genes, gpsA gene, wrbA gene, yieF gene, and fadE gene;
   wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
   wherein the genetically engineered bacteria is able to use oxygen as electron acceptor,
   wherein the bacterium is genetically engineered to produce a fermentation product in presence of oxygen,
   wherein the genetically engineered bacteria is an E. coli.

The genetic engineered bacteria with the desired gene deletions can be obtained by using a gene editing method known in the prior art, such as as classical homologous recombination using restriction enzymes and DNA ligase, lambda Red recombinases system, Rec recombinases system, ET recombination, flippase recombinase, and CRISPR/Cas method, as described in Current Protocol in Molecular Biology, Wiley.

The term “Escherichia coli” (E. coli), as used herein, relates to the well-known Gram-negative, facultative anaerobic, rod-shaped, coliform bacterium of the genus Escherichia. The term “Escherichia coli strain”, (E. coli strain) as used herein, relates to a subgroup within the species that has unique characteristics that distinguish it from other strains. The E. coli K-12 and B strains are well-known and used routinely in molecular biology as both a tool and a model organism. The E. coli strains K-12, B, C, and W are thought of as model organism strains. These are classified in Risk Group 1 in biosafety guidelines. The term “Escherichia coli K-12 strain”, (E. coli K-12 strain) as used herein, relates to an E. coli strain isolated from a stool sample of a patient convalescent from diphtheria and was labelled K-12 in 1922 at Stanford University. The “Escherichia coli K-12 strain MG1655”, Genotype: F- lambda- ilvG- rfb-50 rph-1 Serotype: OR:H48:K—was sequenced by the Blattner laboratory because it approximates wild-type E. coli and “has been maintained as a laboratory strain with minimal genetic manipulation, having only been cured of the temperate bacteriophage lambda and F plasmid by means of ultraviolet light and acridine orange, respectively.” The mutations listed in the genotype are present in most E. coli K-12 strains and were probably acquired early in the history of the laboratory strain. An extensive list of Escherichia coli K-12 strain derivatives and their individual construction, genotypes, phenotypes, plasmids and phage information can be viewed at Ecoliwiki. E. coli MG1655 has been engineered to express the genes encoding an arabinose inducible lambda Red recombineering system and a rhamnose inducible flippase recombinase to allow fast turnover for multiple deletions. However, the present invention is not restricted to a particular E.coli strain and different E. coli strains are suitable for carry out the invention.

Preferred E. coli strains within the scope of the present invention are selected from the group comprising: E. coli MG1655, BW25113 strain, W3110 strain, JM109 strain, B21 strain, BL21(D3) strain, HMS174 strain, RV308(DE3) strain, and AD494 strain.

Thus, the present invention relates to a genetically engineered Escherichia coli as disclosed above, wherein the Escherichia coli is an E. coli MG1655 or an E. coli MG1655 further comprising one or more recombination systems. Preferred recombination systems for the present invention are lambda Red recombinases, Rec recombinases, ET recombination, flippase recombinase.

The term “evolved strain” refers to a bacterial strain that has been cultivated for longer time in culture medium under standard culture conditions selecting for an improvement of growth characteritics. Said process is referred to as “adaptative laboratory evolution”. Usually “adaptative laboratory evolution” requires a prolonged bacterial cultivation.

The recombinase system of lambda Red recombinases comprises three phage-derived lambda Red proteins: Gam, Exo and Beta, which are necessary to complete dsDNA recombination (Murphy, K. C. Use of bacteriophage lambda recombination functions to promote gene replacement in Escherichia coli. J Bacteriol 180, 2063-2071 (1998)); Gam prevents degradation of foreign linear double stranded DNA by the E. coli nucleases, Exo degrades dsDNA to form a single stranded DNA (ssDNA) and Beta binding facilitates recombination. The exact mechanism on how a desired construct recombines with the chromosome in the presence of the three lambda Red proteins has been highly debated.

However, the genetically engineered E. coli of the present invention can be obtained also by other gene editing methods, which are known to the skilled person of the art, such as classical homologous recombination using restriction enzymes and DNA ligase, CRISPR/Cas (Robb, G B 2019. Genome editing with CRISPR-Cas: an overview. Current Protocols Essential Laboratory Techniques, 19, e36).

In alternatively preferred embodiments, the present invention related to a genetically engineered E. coli as described above, wherein the E. coli is an E. coli MG1655. In more preferred embodiments, the present invention related to a genetically engineered E. coli as described above, wherein the E. coli is an E. coli MG1655 further comprising one or more recombination systems selected from the group comprising lambda Red recombinases, Rec recombinases, and flippase recombinase.

### Method for Lactate Production

A second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and
   a deletion of dld gene,
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of a terminal electron acceptor, preferably in presence of oxygen;
   wherein the carbon source is selected from the group comprising glucose, fructose, xylose, arabinose, galactose, mannose, sucrose, cellobiose, hemicellulose, and glycerol or a combination thereof;
   wherein the optional supplement is selected from acetate, pyruvate, and casaminoacids (CAA). Preferably, the fermentation product is D-lactate. Thus, the genetically engineered bacterium produces D-lactate from a carbon source in presence of oxygen.

A second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and
   a deletion of dld gene,
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, hemicellulose, and glycerol or a combination thereof;
   wherein the optional supplement is selected from acetate, pyruvate, and casaminoacids (CAA) Preferably the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

A preferred second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and
   a deletion of dld gene,
   a deletion of one or more genes selected from the group comprising mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of a terminal electron acceptor, preferably in presence of oxygen;
   wherein the carbon source is selected from the group comprising glucose, fructose, xylose, arabinose, galactose, mannose, sucrose, cellobiose, hemicellulose, and glycerol or a combination thereof;
   wherein the supplement is selected from acetate, pyruvate, and casaminoacids (CAA).

Preferably the fermentation product is D-lactate. Thus, the genetically engineered bacterium produces D-lactate from a carbon source in presence of oxygen.

A preferred second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene; and
   a deletion of dld gene,
   a deletion of one or more genes selected from the group comprising mqo gene, gpsA gene, poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, hemicellulose, and glycerol or a combination thereof;
   wherein the supplement is selected from acetate, pyruvate, and casaminoacids (CAA).

Preferably the fermentation product is D-lactate. Thus, the bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Preferably, a second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene; and
   a deletion of glpD gene;
b) providing a culture medium comprising a carbon source and optionally a supplement;
c) culturing the genetically engineered bacteria under aerobic conditions in the culture medium of b),
   wherein the genetically engineered bacterium is able to use oxygen as electron acceptor,
   wherein the bacteria is genetically engineered to produce a fermentation product in presence of a terminal electron acceptor, preferably wherein the bacteria is genetically engineered to produce a fermentation product in presence of oxygen;
   wherein the carbon source is selected from the group comprising glucose, fructose, xylose, arabinose, galactose, mannose, sucrose, cellobiose, and hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

Preferably, a second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene; and
   a deletion of glpD gene;
b) providing a culture medium comprising a carbon source and optionally a supplement;
c) culturing the genetically engineered bacteria under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, and hemicellulose or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

A more preferred second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene;
   a deletion of mqo gene; and
   a deletion of glpD gene;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of a terminal electron acceptor, preferably wherein the bacterium is genetically engineered to produce a fermentation product in presence of oxygen;
   wherein the carbon source is selected from the group comprising glucose, fructose, xylose, arabinose, galactose, mannose, sucrose, cellobiose, and hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

A more preferred second aspect of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene;
   a deletion of mqo gene; and
   a deletion of glpD gene;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, and hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

In other embodiments, the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene; and
   not comprising a glpD deletion;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of a terminal electron acceptor, preferably in presence of oxygen;
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In other embodiments, the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene; and
   not comprising a glpD deletion;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultativ anaerob bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Another embodiment of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene;
   a deletion of mqo gene; and
   not comprising a glpD deletion;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of a terminal electron acceptor, preferably in presence of oxygen;
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

Another embodiment of the present invention relates to a method for obtaining a fermentation product comprising the following steps:
a) providing a genetically engineered bacterium comprising:
   a deletion of ndh gene;
   a deletion of one or more nuo genes, wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene, preferably nuoEFG genes;
   a deletion of dld gene;
   a deletion of mqo gene; and
   not comprising a glpD deletion;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the bacterium is genetically engineered to produce a fermentation product in presence of oxygen;
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Fermentation Method with NNmini Strain +gplD

A further aspect of the present invention relates to a method for obtaining a fermentation product comprising the following steps:
a) providing a genetically engineered bacterium comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene;
b) providing a culture medium comprising a carbon source and optionally a supplement;
c) culturing the genetically engineered bacterium under aerobic conditions in the culture medium of b),
   wherein the bacterium is genetically engineered to produce lactate from glycerol in presence of a terminal electron acceptor, preferably in presence of oxygen,
   wherein the genetically engineered bacteria is able to use oxygen as electron acceptor,
   wherein the carbon source is selected from the group comprising glucose, fructose, xylose, arabinose, galactose, mannose, sucrose, cellobiose, hemicellulose, and glycerol or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

A further aspect of the present invention relates to a method for obtaining a fermentation product comprising the following steps:
a) providing a genetically engineered bacterium comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene;
b) providing a culture medium comprising a carbon source and optionally a supplement;
c) culturing the genetically engineered bacterium under aerobic conditions in the culture medium of b),
   wherein the bacterium is genetically engineered to produce lactate from glycerol in presence of oxygen,
   wherein the genetically engineered bacteria is able to use oxygen as electron acceptor,
   wherein the carbon source is selected from the group comprising glucose, fructose, xylose, arabinose, galactose, mannose, sucrose, cellobiose, hemicellulose, and glycerol or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

The present invention further relates to a method for obtaining a fermentation product comprising the following steps:
a) providing a genetically engineered bacteria comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the carbon source is selected from the group comprising glucose, fructose, xylose, arabinose, galactose, mannose, sucrose, cellobiose, hemicellulose, and glycerol or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

More preferably, the present invention relates to a method for obtaining a fermentation product comprising the following steps:
a) providing a genetically engineered bacterium comprising a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, of yieF gene, and of fadE gene, and not comprising a glpD deletion;
b) providing a culture medium comprising a carbon source and optionally a supplement;
c) culturing the genetically engineered bacterium under aerobic conditions in the culture medium of b),
   wherein the bacteria is genetically engineered to produce D-lactate from glycerol in presence of a terminal electron acceptor, preferably in presence of oxygen,
   wherein the genetically engineered bacterium is able to use oxygen as electron acceptor,
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA),

Preferably, the fermentation product is D-lactate. Thus, the genetically engineered bacterium produces D-lactate from a carbon source in presence of oxygen.

More preferably, the present invention relates to a method for obtaining a fermentation product comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, and fadE gene, and not comprising a glpD deletion;
b) providing a culture medium comprising a carbon source and optionally a supplement;
c) culturing the genetically engineered bacterium under aerobic conditions in the culture medium of b),
   wherein the bacterium is genetically engineered to produce D-lactate from glycerol in presence of a terminal electron acceptor, preferably in presence of oxygen,
   wherein the genetically engineered bacterium is able to use oxygen as electron acceptor,
   wherein the carbon source is selected from the group comprising glucose, fructose, xylose, arabinose, galactose, mannose, sucrose, cellobiose, hemicellulose, and glycerol or a combination thereof, preferably wherein the carbon source is glycerol;
   wherein the supplement is selected from acetate, pyruvate, and casaminoacids (CAA); Preferably, the fermentation product is D-lactate. Thus, the genetically engineered bacterium produces D-lactate from a carbon source in presence of oxygen.

In more preferred embodiments, the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In still more preferred embodiments, the genetically engineered bacteria is an E. coli.

Fermentation Method with NNmini Strain +gpsA

Preferably, the present invention relates to a method for obtaining a fermentation product comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene;
b) providing a culture medium comprising a carbon source and optionally a supplement;
c) culturing the genetically engineered bacterium under aerobic conditions in the culture medium of b),
   wherein the bacterium is genetically engineered to produce D-lactate from glucose in presence of a terminal electron acceptor, preferably in presence of oxygen,
   wherein the genetically engineered bacterium is able to use oxygen as electron acceptor,
   wherein the carbon source is selected from the group comprising glucose, fructose, xylose, arabinose, galactose, mannose, sucrose, cellobiose, hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

Preferably, the present invention relates to a method for obtaining a fermentation product comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene;
b) providing a culture medium comprising a carbon source and optionally a supplement;
c) culturing the genetically engineered bacterium under aerobic conditions in the culture medium of b),
   wherein the bacterium is genetically engineered to produce D-lactate from glucose in presence of oxygen,
   wherein the genetically engineered bacteria is able to use oxygen as electron acceptor,
   wherein the carbon source is selected from the group comprising glucose, fructose, xylose, arabinose, galactose, mannose, sucrose, cellobiose, and hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

Preferably, the present invention relates to a method for obtaining a fermentation product comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, glpD gene, wrbA gene, yieF gene, and fadE gene;
b) providing a culture medium comprising a carbon source and optionally a supplement;
c) culturing the genetically engineered bacterium under aerobic conditions in the culture medium of b),
   wherein the genetically engineered bacterium is able to use oxygen as electron acceptor,
   wherein the carbon source is selected from the group comprising glucose, fructose, xylose, arabinose, galactose, mannose, sucrose, cellobiose, and hemicellulose, or a combination thereof;
   wherein the supplement is selected from acetate, pyruvate, and casaminoacids (CAA).

Preferably, the fermentation product is D-lactate. Thus, the genetically engineered bacterium produces D-lactate from a carbon source in presence of oxygen.

In more preferred embodiments, the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In still more preferred embodiments, the genetically engineered bacterium is an E. coli.

### Isobutanol Producing Genetically Engineered Bacteria and Method for Producing Isobutanol.

A preferred embodiment of the present invention relates to a genetically engineered bacterium able to produce isobutanol from glycerol in presence of oxygen,
wherein the genetically engineered bacteria is able to use oxygen as electron acceptor,
wherein the genetically engineered bacteria comprises a deletion of ndh gene, nuoEFG genes, dld gene, mqo gene, poxB gene, sdhABCD genes, glcDEF genes, hgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacteria further comprises an engineered metabolic pathway for converting pyruvate to isobutanol, wherein said engineered metabolic pathway comprises AlsS, IlvC, IlvD, KivD and AdhA. Preferably, the genetically engineered bacterium produces isobutanol and/or ethanol, preferably isobutanol from a carbon source in presence of oxygen.

A more preferred embodiment of the present invention relates to a genetically engineered bacterium able to produce isobutanol from glycerol in presence of oxygen,
wherein the genetically engineered bacteria is able to use oxygen as electron acceptor,
wherein the genetically engineered bacteria comprises a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacteria further comprises an engineered metabolic pathway for converting pyruvate to isobutanol, wherein said engineered metabolic pathway comprises AlsS, IlvC, IlvD, KivD and AdhA, and
wherein the genetically engineered bacteria is derived from a facultative anaerobic bacterium selected from the group comprising Enterobacteriaceae, Staphylococcaceae, Streptococcaceae, Lactobacillaceae, Vibrionaceae, and Pasteurellaceae. Preferably, the genetically engineered bacterium produces isobutanol and/or ethanol, preferably isobutanol from a carbon source in presence of oxygen.

A further preferred embodiment of the present invention relates to a genetically engineered bacterium able to produce isobutanol from glycerol in presence of oxygen,
wherein the genetically engineered bacterium is able to use oxygen as electron acceptor,
wherein the genetically engineered bacterium comprises a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is an E. coli. Preferably, the genetically engineered bacterium produces isobutanol and/or ethanol, preferably isobutanol from a carbon source in presence of oxygen.

Another preferred embodiment of the present invention is directed to a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is able to produce isobutanol from glycerol in presence of a terminal electron acceptor, preferably in presence of oxygen,
and wherein the genetically engineered bacterium is able to use oxygen as electron acceptor. Preferably, the genetically engineered bacterium produces isobutanol and/or ethanol, preferably isobutanol from a carbon source in presence of oxygen.

In more preferred embodiments, the genetically engineered bacteria is derived from a facultativ anaerob bacteria selected from the group comprising Enterobacteriaceae, Staphylococcaceae, Streptococcaceae, Lactobacillaceae, Vibrionaceae, and Pasteurellaceae.

Another further preferred embodiment of the present invention is directed to a genetically engineered E. coli bacterium comprising a deletion ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacteria is able to produce isobutanol from glycerol in presence of a terminal electron acceptor, preferably in presence of oxygen,
and wherein the genetically engineered bacteria is able to use oxygen as electron acceptor. Preferably, the genetically engineered bacterium produces isobutanol and/or ethanol, preferably isobutanol from a carbon source in presence of oxygen.

Thus, another further preferred embodiment of the present invention is directed to a genetically engineered E. coli bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is able to produce isobutanol from glycerol in presence of oxygen,
and wherein the genetically engineered bacterium is able to use oxygen as electron acceptor. Preferably, the genetically engineered bacterium produces isobutanol and/or ethanol, preferably isobutanol from a carbon source in presence of oxygen.

Without mutations or genetic modifications, E. coli cannot produce ethanol under aerobic conditions because its native main alcohol dehydrogenase adhE is inhibited by oxygen. For the enzyme to be active under aerobic conditions, 2 mutations would be necessary (5′UTR of adhE and Glu568Lys). Thus, one way to further direct the fermentative production of ethanol would be to include the genetic modifications described above (5′UTR of adhE and Glu568Lys, such as described in https://doi.org/10.1128/jb.182.21.6049-6054.2000 (Holland-Staley C A, Lee K, Clark D P, Cunningham P R 2000. Aerobic Activity of Escherichia coli Alcohol Dehydrogenase Is Determined by a Single Amino Acid. J Bacteriol 1829) or to overexpress any other oxygen-tolerant ethanol bioproduction pathway (e.g. such as described in https://doi.org/10.1128/aem.53.10.2420-2425.1987 Ingram L O, Conway T, Clark D P, Sewell G W, Preston J F. 1987. Genetic engineering of ethanol production in Escherichia coli. Appl Environ Microbiol 53).

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol;
c) culturing the genetically engineered bacterium under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacteria is able to produce isobutanol from glycerol in presence of a terminal electron acceptor, preferably in presence of oxygen. Thus, the genetically engineered bacterium produces isobutanol and/or ethanol, preferably isobutanol from a carbon source in presence of oxygen.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol;
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glycerol in presence of oxygen. Thus, the genetically engineered bacterium produces isobutanol and/or ethanol, preferably isobutanol from a carbon source in presence of oxygen.

A more preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol;
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and wherein the genetically engineered bacteria is able to produce isobutanol from glycerol in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. Thus, the genetically engineered bacterium produces isobutanol and/or ethanol, preferably isobutanol from a carbon source in presence of oxygen.

A further preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol;
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and wherein the genetically engineered bacterium is able to produce isobutanol from glycerol in presence of oxygen, wherein the genetically engineered bacteria is an E. coli.

Thus, the genetically engineered bacterium produces isobutanol and/or ethanol, preferably isobutanol from a carbon source in presence of oxygen.

Moreover, the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol; and a supplement selected from acetate, pyruvate, and casaminoacids (CAA);
c) culturing the genetically engineered bacterium under aerobic conditions in the culture medium of b) to produce a culture medium comprising isobutanol and/or ethanol, preferably isobutanol.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and ldhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol; and a supplement selected from acetate, pyruvate, and casaminoacids (CAA);
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glycerol in presence of a terminal electron acceptor, preferably wherein the bacteria is genetically engineered to produce a fermentation product in presence of oxygen,
   wherein the genetically engineered bacteria is derived from a facultativ anaerob bacteria selected from the group comprising Enterobacteriaceae, Staphylococcaceae, Streptococcaceae, Lactobacillaceae, Vibrionaceae, and Pasteurellaceae. Thus, the genetically engineered bacterium produces isobutanol and/or ethanol, preferably isobutanol from a carbon source in presence of oxygen.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol; and a supplement selected from acetate, pyruvate, and casaminoacids (CAA);
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glycerol in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. Thus, the genetically engineered bacterium produces isobutanol and/or ethanol, preferably isobutanol from a carbon source in presence of oxygen.

A further aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol; and a supplement selected from acetate, pyruvate, and casaminoacids (CAA);
c) culturing the genetically engineered bacterium under aerobic conditions in the culture medium of b) to produce a culture medium comprising isobutanol and/or ethanol;
   wherein the genetically engineered bacterium is able to use oxygen as electron acceptor, and
   wherein the genetically engineered bacteria is able to produce isobutanol from glycerol in presence of a terminal electron acceptor, preferably wherein the bacterium is genetically engineered to produce a fermentation product in presence of oxygen,
   wherein the genetically engineered bacterium is an E. coli. Thus, the genetically engineered bacterium produces isobutanol and/or ethanol, preferably isobutanol from a carbon source in presence of oxygen.

A further aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gpsA gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, hemicellulose, and glycerol or a combination thereof, preferably wherein the carbon source is selected from glucose and glycerol or a combination thereof, preferably, more preferably the carbon source is glycerol; and a supplement selected from acetate, pyruvate, and casaminoacids (CAA);
c) culturing the genetically engineered bacterium under aerobic conditions in the culture medium of b) to produce a culture medium comprising isobutanol and/or ethanol;
   wherein the genetically engineered bacterium is able to use oxygen as electron acceptor, and wherein the genetically engineered bacterium is able to produce isobutanol from glycerol in presence of oxygen, wherein the genetically engineered bacterium is an E. coli.

Thus, the genetically engineered bacterium produces isobutanol and/or ethanol, preferably isobutanol from a carbon source in presence of oxygen.

A further embodiment of the present invention relates to a genetically engineered bacterium able to produce isobutanol from glycerol in presence of oxygen,
wherein the genetically engineered bacteria is able to use oxygen as electron acceptor,
wherein the genetically engineered bacteria comprises a deletion of ndh gene, nuoEFG genes, dld gene, mqo gene, poxB gene, sdhABCD genes, glcDEF genes, hgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacteria further comprises an engineered metabolic pathway for converting pyruvate to isobutanol, wherein said engineered metabolic pathway comprises AlsS, IlvC, IlvD, KivD and AdhA. In some embodiments, the genetically engineered bacterium further comprises a deletion of gpsA gene.

A more preferred embodiment of the present invention relates to a genetically engineered bacterium able to produce isobutanol from glycerol in presence of oxygen,
wherein the genetically engineered bacteria is able to use oxygen as electron acceptor,
wherein the genetically engineered bacteria comprises a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacteria is derived from a facultative anaerobic bacterium selected from the group comprising Enterobacteriaceae, Staphylococcaceae, Streptococcaceae, Lactobacillaceae, Vibrionaceae, and Pasteurellaceae. In some embodiments, the genetically engineered bacterium further comprises a deletion of gpsA gene.

A further preferred embodiment of the present invention relates to a genetically engineered bacterium able to produce isobutanol from glycerol in presence of oxygen,
wherein the genetically engineered bacterium is able to use oxygen as electron acceptor,
wherein the genetically engineered bacterium comprises a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is an E. coli. In some embodiments, the genetically engineered bacterium further comprises a deletion of gpsA gene.

Another preferred embodiment of the present invention is directed to a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is able to produce isobutanol from glucose in presence of a terminal electron acceptor, preferably in presence of oxygen,
and wherein the genetically engineered bacterium is able to use oxygen as electron acceptor. In some embodiments, the genetically engineered bacterium further comprises a deletion of gpsA gene. In more preferred embodiments, the genetically engineered bacteria is derived from a facultativ anaerob bacteria selected from the group comprising Enterobacteriaceae, Staphylococcaceae, Streptococcaceae, Lactobacillaceae, Vibrionaceae, and Pasteurellaceae.

Another further preferred embodiment of the present invention is directed to a genetically engineered E. coli bacterium comprising a deletion ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacteria is able to produce isobutanol from glucose in presence of a terminal electron acceptor, preferably in presence of oxygen,
and wherein the genetically engineered bacteria is able to use oxygen as electron acceptor. In some embodiments, the genetically engineered bacterium further comprises a deletion of gpsA gene.

Thus, another further preferred embodiment of the present invention is directed to a genetically engineered E. coli bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and
wherein the genetically engineered bacterium further comprises an engineered metabolic pathway for converting pyruvate to isobutanol, wherein said engineered metabolic pathway comprises AlsS, IlvC, IlvD, KivD and AdhA, and
wherein the genetically engineered bacterium is able to produce isobutanol from glucose in presence of oxygen,
and wherein the genetically engineered bacterium is able to use oxygen as electron acceptor. In some embodiments, the genetically engineered bacterium further comprises a deletion of gpsA gene.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose;
c) culturing the genetically engineered bacterium under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glucose in presence of a terminal electron acceptor, preferably in presence of oxygen. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose;
c) culturing the genetically engineered bacteria under aerobic conditions in the culture medium of b) to produce a culture medium comprising isobutanol and/or ethanol, preferably isobutanol;
   wherein the genetically engineered bacterium is able to use oxygen as electron acceptor, and wherein the genetically engineered bacterium is able to produce isobutanol from glucose in presence of oxygen. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A more preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose;
c) culturing the genetically engineered bacterium under aerobic conditions in the culture medium of b) to produce a culture medium comprising isobutanol and/or ethanol, preferably isobutanol;
   wherein the genetically engineered bacteria is able to use oxygen as electron acceptor, and wherein the genetically engineered bacterium is able to produce isobutanol from glucose in presence of oxygen,
   wherein the genetically engineered bacteria is derived from a facultativ anaerob bacteria selected from the group comprising Enterobacteriaceae, Staphylococcaceae, Streptococcaceae, Lactobacillaceae, Vibrionaceae, and Pasteurellaceae. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A further preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose;
c) culturing the genetically engineered bacterium under aerobic conditions in the culture medium of b) to produce a culture medium comprising isobutanol and/or ethanol, preferably isobutanol;
   wherein the genetically engineered bacterium is able to use oxygen as electron acceptor, and wherein the genetically engineered bacteria is able to produce isobutanol from glucose in presence of oxygen, wherein the genetically engineered bacterium is an E. coli. In some embodiments, the genetically engineered bacterium further comprises a deletion of gpsA gene. Thus, the genetically engineered bacterium produces isobutanol and/or ethanol, preferably isobutanol from a carbon source in presence of oxygen.

Moreover, the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose; and a supplement selected from acetate, pyruvate, and casaminoacids (CAA);
c) culturing the genetically engineered bacterium under aerobic conditions in the culture medium of b) to produce a culture medium comprising isobutanol and/or ethanol, preferably isobutanol.

In some embodiments, the genetically engineered bacterium further comprises a deletion of gpsA gene. Thus, the genetically engineered bacterium produces isobutanol and/or ethanol, preferably isobutanol from a carbon source in presence of oxygen.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and ldhA gene, and further comprising an engineered metabolic pathway for converting pyruvate to isobutanol, wherein said engineered metabolic pathway comprises AlsS, IlvC, IlvD, KivD and AdhA;
b) prov providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose; and a supplement selected from acetate, pyruvate, and casaminoacids (CAA);
c) culturing the genetically engineered bacterium under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glucose in presence of a terminal electron acceptor, preferably wherein the bacteria is genetically engineered to produce a fermentation product in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A preferred aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose; and a supplement selected from acetate, pyruvate, and casaminoacids (CAA);
c) culturing the genetically engineered bacterium under aerobic conditions in the culture medium of b) to produce a culture medium comprising isobutanol and/or ethanol, preferably isobutanol;
   wherein the genetically engineered bacterium is able to use oxygen as electron acceptor, and
   wherein the genetically engineered bacterium is able to produce isobutanol from glucose in presence of oxygen,
   wherein the genetically engineered bacterium is derived from a facultativ anaerob bacteria selected from the group comprising Enterobacteriaceae, Staphylococcaceae, Streptococcaceae, Lactobacillaceae, Vibrionaceae, and Pasteurellaceae. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A further aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose; and a supplement selected from acetate, pyruvate, and casaminoacids (CAA);
c) culturing the genetically engineered bacterium under aerobic conditions in the culture medium of b) to produce a culture medium comprising isobutanol and/or ethanol;
   wherein the genetically engineered bacterium is able to use oxygen as electron acceptor, and
   wherein the genetically engineered bacterium is able to produce isobutanol from glucose in presence of a terminal electron acceptor, preferably wherein the bacterium is genetically engineered to produce a fermentation product in presence of oxygen,
   wherein the genetically engineered bacterium is an E. coli. In some embodiments, the genetically engineered bacterium further comprises a deletion of gpsA gene. Thus, the genetically engineered bacterium produces isobutanol and/or ethanol, preferably isobutanol from a carbon source in presence of oxygen.

A further aspect of the present invention relates to a method for producing isobutanol and/or ethanol comprising the following steps:
a) providing a genetically engineered bacterium comprising a deletion of ndh gene, nuoEFG genes, dld gene, mqo gene, poxB gene, sdhABCD genes, glcDEF genes, lhgO gene, putA gene, lldD gene, dadA gene, kefF gene, mdaB-ygiN gene, glpABC genes, gplD gene, wrbA gene, yieF gene, fadE gene, and IdhA gene, and further comprising an engineered metabolic pathway for converting pyruvate to isobutanol, wherein said engineered metabolic pathway comprises AlsS, IlvC, IlvD, KivD and AdhA;
b) providing a culture medium comprising wherein a carbon source selected from the group comprising glucose, fructose, xylose, arabinose, galactose, mannose, sucrose, cellobiose, and hemicellulose, or a combination thereof, preferably wherein the carbon source is glucose; and a supplement selected from acetate, pyruvate, and casaminoacids (CAA);
c) culturing the genetically engineered bacterium under aerobic conditions in the culture medium of b) to produce a culture medium comprising isobutanol and/or ethanol;
   wherein the genetically engineered bacterium is able to use oxygen as electron acceptor, and wherein the genetically engineered bacterium is able to produce isobutanol from glucose in presence of oxygen, wherein the genetically engineered bacterium is an E. coli.

In some embodiments, the genetically engineered bacterium further comprises a deletion of gpsA gene. Thus, the genetically engineered bacterium produces isobutanol and/or ethanol, preferably isobutanol from a carbon source in presence of oxygen.

### DESCRIPTION OF THE FIGURES

FIG. 1 shows lactate production from glucose by the NNmini strain. Measurements represent means and errors from three independent experiments.

FIG. 2 shows lactate production from glycerol by the NNmini +glpD strain. Measurements represent means and errors from three independent experiments.

FIG. 3 shows A) genes deleted in the NNmini strain and illustrates their respective functions, e.g. NAD(P)H-dehydrogenases and quinone dependent dehydrogenases involved in either mini-cycles or central metabolism in the NNmini strain. B) Schematic representation of electron fluxes (blue arrows) during cellular respiration in the wild type (left) and the aerobic fermentative NNmini strain (right). Due to disruptions of all reactions allowing electron transfer from reducing equivalents to quinones (indicated by red crosses), the strain relies on the secretion of fermentation products for redox balance maintenance.

FIG. 4 shows A) comparison of aerobic lactate production from glucose using the E. coli strains ΔubiCA, NNQ, and NNmini. The data shown represents means of three independent measurements. The strains were incubated in M9 minimal medium under ambient (aerobic) conditions in the presence of 20, 10, 5, 2.5, 1.25, 0.6, 0.3, 0.15 mM glucose (NNQ and NNmini) or in presence of 20, 11, 6.9, 4.7 mM glucose (ΔubiCA). After the cultures reached stationary growth phase, supernatant samples were taken and analysed by ion-chromatography. B) Growth of strain NNminimal1 in M9 medium with different glucose concentrations. C) Comparison of lactate production from 40 mM glucose of NNmini and NNminimal1.

FIG. 5 shows A) comparison of aerobic acetate production from glucose using the following E. coli strains: (i) deleted in ubiquinone biosynthesis (ΔubiCA), (ii) deleted in both NADH dehydrogenases and ubiquinone biosynthesis (NNQ), and (iii) deleted in both NADH dehydrogenases as well as in reactions which might bypass NADH-dehydrogenase activity as described in Example 3, 4, Table 1, 3, and shown in FIG. 3 (NNmini). The data shown represents means of three independent measurements. B) Growth of a wildtype control strain (right), the ΔubiCA strain (middle) and the NNQ strain (left) on 10 mM glucose (blue) or 20 mM glycerol (pink). C) Both NNQ (green bullets) and ΔubiCA strain (yellow diamonds) grow to lower biomass yields, indicated by maximum OD₆₀₀, than the wild type (purple squares) with glucose as sole carbon source. Mean values of duplicate measurements are plotted with error bars. D) Fermentation products detected in culture supernatants from the ΔubiCA and the NNQ strain grown on glucose. Mean values of duplicate measurements are shown with error bars.

FIG. 6 shows comparison of aerobic lactate production from glycerol using the E. coli strains ΔubiCA, NNQ, and NNmini with reintroduced chromosomal glpD, i.e. NNmini glpD⁺. The data represent means of three independent measurements. The strains were incubated in M9 minimal medium under ambient (aerobic) conditions in the presence of 40, 20, 10, 5, 2.5, 1.25, 0.6, 0.3 mM glycerol (NNmini +glpD and NNQ) or 40, 23.5, 13.8, 8.1 mM glycerol (ΔubiCA). After the cultures reached stationary growth phase, supernatant samples were taken and analysed by ion-chromatography to determine concentrations of lactate and acetate.

FIG. 7 shows comparison of aerobic acetate production from glycerol using the following E. coli strains ΔubiCA, NNQ, and NNmini with reintroduced chromosomal glpD, i.e. NNmini glpD⁺. The data represent means of three independent measurements. Incubations were performed as described for FIG. 6.

FIG. 8 shows growth on glucose or glycerol comprising minimal media of WT (A), NNQ (B), NNQ1 Ev.S (isolated evolved NNQ strain 1S) (C), NNQ1 Ev.B (isolated evolved NNQ strain 1B) (D), and of the reverse engineered strain NNQ ΔubiE1 (mutation in did promotor) (E) and NNQ ΔubiE2 (no mutation in did promotor) (F), mutants NNQ2 Ev.S (isolated evolved NNQ strain 1S) (G), NNQ2 Ev.B (isolated evolved NNQ strain 1B) (H), (1) The evolved NNQ strain grows to higher biomass yields with glucose (blue) and glycerol (pink). (L) A knockout of the menaquinone biosynthesis genes is lethal for the evolved NNQ strain. (M) Retro-engineering of the discovered mutations in the evolved NNQ strain allow respiratory growth of an unevolved NNQ strain. (N) Mutations/Deletion of ubiE is known to result in accumulation of demethylmenaquinol.

FIG. 9 shows characterization of the NNmini strain in aerobic and anaerobic conditions. A, B) Growth of the NNmini strain (B) and a wild-type strain (A) on varying glucose concentrations. C) The NNmini strain grows to much lower biomass yields, indicated by lower OD600 values, than the wild type. The mean of duplicate measurements is shown with error bars. D) Lactate concentrations detected in cultivation supernatants of the NNmini strain grown aerobically in minimal medium with varying glucose concentrations. For the wild type, no lactate was detected can therefore not be represented in a log scale. The mean of duplicate measurements is shown with error bars. E, F) Anaerobic growth of the NNmini strain and a wild type in minimal medium supplemented with different glucose concentrations. G) Comparison of fermentation products from wild type and NNmini grown anaerobically on 10 mM glucose. The mean of duplicate measurements is shown with error bars.

FIG. 10 shows aerobic characterization of an NAD(P)H dehydrogenase deficient strain. A, B) Comparison of growth phenotype on different carbon sources of an NADPH deficient strain (ΔnuoEFG Δndh) with a wild type. On acetate, growth is abolished for the ΔnuoEFG Δndh strain while growth on the other carbon sources exhibits an extended lag phase and lower biomass yields. C) Lactate production (squares) and OD₆₀₀ (bullets) was measured during the aerobic cultivation of the ΔnuoEFG Δndh strain on 40 mM glucose. Mean values of triplicate measurements are shown with error bars.

FIG. 11 shows aerobic glycerol fermentation by the NNmini +glpD strain. A) Schematic representation of glycerol metabolism in the NNmini +glpD strain. Reintroduction of glpD allows transferring one electron pair to ubiquinone, which re-balances the fermentation of glycerol to lactate. B) Growth of the engineered NNmini and the NNmini +glpD strain on minimal medium supplemented with varying glycerol concentrations (indicated to the right). C) Biomass yields, indicated by maximum OD₆₀₀ values, of the NNmini +glpD strain are lower than those of a wild type grown with glycerol as sole carbon source. D) Lactate detected in supernatants of the NNmini +glpD strain grown with varying glycerol concentrations. The mean of duplicate measurements is shown with error bars. E) NNmini strain with glpD deletion is unable to grow on glycerol. Glycerol concentrations are indicated by the legend to the right.

FIG. 12 shows improvement of growth of the NNmini strain by supplementation of acetate, pyruvate or casamino acids. A) Growth of the NNmini strain on 10 mM glucose with different acetate concentrations. 20 mM malate (non-fermentable)+10 mM acetate were used as negative control medium to demonstrate that acetate itself would not allow the NNmini strain to grow (data not shown). B) Comparison of the growth-improving effect from feeding different acetate, pyruvate or casamino acid (CAA) concentrations. Values are given in g/L to allow comparison with CAAs, which are a complex medium without defined molecular weight. C) Schematic view of expected carbon flow towards selected biomass precursors in NNmini strain grown on glucose with acetate supplementation. Since malate dehydrogenase (Mqo) and succinate dehydrogenase (Sdh) are absent, acetate can only be used for the generation of biomass precursors from lower metabolism. Carbons from acetate are therefore expected to be found in metabolites colored in orange. Of these, the amino acids leucine (made partly from pyruvate and acetyl-CoA), proline and arginine (both derived from a-KG) are expected to carry carbons from acetate, in contrast to amino acids from upper metabolism. D) Schematic view of the enzymes involved in the conversion of pyruvate into acetyl-CoA. Pfl is oxygen-sensitive and can therefore only operate under anaerobic conditions. Pdh produces NADH and is inhibited by it. E) Isotopic labelling patterns found in alanine, serine, aspartate, leucine, proline and arginine obtained from the NNmini strain grown on 20 mM glucose+10 mM ¹³C₂-acetate. Only acetyl-CoA derived leucine and a-KG derived proline and arginine are double labelled. F) Supplementation of a pyruvate gradient to NNmini strain growing aerobically on 10 mM glucose. G) Supplementation of a casamino acid (CAA) gradient to NNmini strain growing aerobically on 10 mM glucose. Abbreviations: a-KG—α-ketoglutarate, 3-PG—3-phosphoglycerate, Pfl—pyruvate formate lyase, Pdh—pyruvate dehydrogenase.

FIG. 13 shows re-balanced, conversion of glycerol to isobutanol by the NNmini ΔldhA +glpD +pIBA strain. A) Schematic representation of glycerol fermentation to isobutanol. By deleting the native lactate synthesis route (ΔldhA), the metabolic flux can be re-directed towards isobutanol. The genes for isobutanol synthesis are expressed from the pIBA plasmid. The isobutanol pathway scheme was adapted from Ghosh et al. 2019. B) IPTG-dependent growth of the NNmini ΔldhA +glpD +pIBA strain grown with glycerol as sole carbon source and acetate supplementation. Here, acetate served as a negative control in addition to the “no IPTG” condition. C) Fermentation products detected in the supernatants of the NNmini ΔldhA +glpD +pIBA strain grown with different glycerol concentrations alone (graph on the right) or supplemented with 10 mM acetate or 5 g/L CAAs. The mean values of triplicate measurements are shown with error bars.

FIG. 14 shows cellular electron flows during respiration and fermentation. A) Schematic representation of the electron transport chain with highlighted engineering targets to achieve aerobic fermentation. Electrons are transferred (blue arrows) from reducing equivalents to the NADH dehydrogenases (blue, Ndh and Nuo) and quinone dehydrogenases (orange, either NAD(P)H dependent ones or others, for example the succinate dehydrogenase). Quinones serve as intermediate electron carrier until oxygen serves as final electron acceptor for the terminal oxidases (green). Nuo and the terminal oxidases are generating a proton (H⁺) gradient which powers the ATPase (violet). This transfer allows quinone reduction to quinol; transfer through the respiratory chain is indicated by blue arrows. Abbreviations: H⁺, Q/QH₂—quinone/quinole. B) Stoichiometry of homolactic fermentations. Left: balanced fermentation of glucose; middle: unbalanced fermentation of glycerol; right: re-balanced respiro-fermentation of glycerol through the quinone-dependent glycerol 3-phosphate dehydrogenase (encoded by glpD)

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### EXAMPLES

### Methods

Strain and plasmids construction. All E. coli strains used in this study are listed in Table 1. An E. coli MG1655 further expressing a λ-red recombinase system and a flippase recombinase, E. coli SIJ_488, was a gift from Alex Nielesen (Addgene plasmid #68246) and used as wildtype (E. coli WT). Gene deletions were performed by λ-Red recombineering or P1-transduction.

E. coli culture media. LB medium (1% NaCl, 0.5% yeast extract, 1% tryptone) was used for cloning, generation of deletion strains, and strain maintenance. When appropriate, kanamycin (25 μg/mL), ampicillin (100 μg/mL), streptomycin (100 μg/mL), or chloramphenicol (30 μg/mL) were used. Growth experiments were carried out without antibiotics in standard M9 minimal medium free of any carbon source or amino acids, and containing 50 mM Na₂HPO₄, 20 mM KH₂PO₄, 1 mM NaCl, 20 mM NH₄Cl, 2 mM MgSO₄ and 100 μM CaCl₂), 134 μM EDTA, 13 μM FeCl₃·6H₂O, 6.2 μM ZnC₂, 0.76 μM CuCl₂·2H₂O, 0.42 μM CoCl₂·2H₂O, 1.62 μM H3BO₃, 0.081 μM MnCl₂·4H₂O. Carbon sources were added as indicated in the Examples and Figures.

### Growth Analysis of Mutant Strains NNmini, NNminimal1, NNminimal2 and NNQ

Overnight cultures of NNmini, NNminimal1, NNminimal2 and NNQ were incubated in 4 mL M9 medium containing 20 mM glucose supplemented with 10 mM acetate. Cultures were harvested (6,000*g, 3 min) and washed three times in M9 medium to remove residual carbon sources. For growth analysis the washed cells were inoculated to an OD₆₀₀ of 0.01 in 96-well microtiter plates (Nunclon Delta Surface, Thermo Scientific) at 37° C. or 30° C. for isobutanol production experiments. Each well contained 150 μL of culture and to avoid evaporation while allowing gas exchange 50 μL mineral oil (Sigma-Aldrich). Growth in technical triplicates was monitored at 37° C. or 30° C. in a BioTek Epoch 2 Microtiterplate reader (BioTek, Bad Friedrichshall, Germany) by absorbance measurements (600 nm) of each well every ~10 minutes with intermittent orbital and linear shaking. As previously established empirically for the instruments, blank measurements were subtracted and OD₆₀₀ measurements were converted to cuvette OD₆₀₀ values by multiplying with a factor of 4.35. Growth curves represent averages of measurements of technical replicates and were plotted in MATLAB and python.

Analysis of supernatants with Ion Chromatography. For detection of fermentation products, supernatants were analyzed by ion chromatography using the Dionex IonPac AS11-HC-4 μm Analytical/Capillary Colum (Thermo Scientific, Dreieich, Germany) with suppressed conductivity detection. Standards of D-lactate, acetate and formate were prepared with deionized water. Supernatants from cell cultures were isolated by centrifugation for 10 minutes at 20,000*g and transfer of the supernatant to a new Eppendorf tube. Both standards and supernatants were diluted 100-fold with water. The analytes in a 10 μl injection were separated using electrolytically generated potassium hydroxide eluent from 1 to 60 mM KOH at 0.38 ml/min within 53 min at 10° C.

Analysis of ΔNuo ΔNdh Cultivation Supernatants with IDMS.

Lactate in Δnuo Δndh culture supernatants was quantified using isotope dilution mass spectrometry (IDMS). The chromatographic separation was performed with 2 μl injection volume on an Agilent Infinity II 1290 HPLC system using a Kinetex EVO C18 column (150×2.1 mm, 3 μm particle size, 100 Å pore size, Phenomenex) connected to a guard column of similar specificity (20×2.1 mm, 3 μm particle size, Phenomoenex) with a constant flow rate of 0.2 ml/min with mobile phase A being 0.1% formic acid in water and phase B being 0.1% formic acid methanol (Honeywell, Morristown, New Jersey, USA) at 25° C. The mobile phase profile consisted of the following steps and linear gradients: 0-4 min constant at 0% B; 4-6 min from 0 to 100% B; 6-7 min constant at 100% B; 7-7.1 min from 100 to 0% B; 7.1 to 12 min constant at 0% B. An Agilent 6495 ion funnel mass spectrometer was used in negative mode with an electrospray ionization source and the following conditions: ESI spray voltage 2000 V, nozzle voltage 500 V, sheath gas 250° C. at 11 l/min, nebulizer pressure 50 psig and drying gas 80° C. at 16 l/min. Compounds were identified based on their mass transition and retention time compared to standards. Chromatograms were integrated using MassHunter software (Agilent, Santa Clara, CA, USA). Absolute concentrations were calculated based on an external calibration curve and corrected for matrix effects by the use of u-13C-lactate as internal standard. Mass transitions, collision energies, Cell accelerator voltages and Dwell times have been optimized using chemically pure standards. The parameter settings of all targets are given in table 4.

TABLE 4
Parameter settings for IDMS measurement targets.

| | Prec | Prod | Dwell | Fragmentor | Collision | Cell | |
|---|---|---|---|---|---|---|---|
| Compound | Ion m/z | Ion m/z | time | voltage | Energy | Accelerator | |
| Name | (Da/eV) | (Da/eV) | (msec) | (V) | (AU) | (AU) | Polarity |
| Lactate IS | 92 | 92 | 150 | 380 | 0 | 5 | Negative |
| Quantifier | | | | | | | |
| Lactate IS | 92 | 74 | 150 | 380 | 10 | 5 | Negative |
| Qualifier | | | | | | | |
| Lactate | 89 | 89 | 150 | 380 | 0 | 5 | Negative |
| Quantifier | | | | | | | |
| 89 | | | | | | | |
| Lactate | 89 | 71 | 150 | 380 | 10 | 5 | Negative |
| Qualifier | | | | | | | |
| 89 | | | | | | | |

Analysis of supernatants with High-Performance Anion-Exchange Chromatography. For detection of alcohols and sugars, supernatants were analyzed by high-performance anion exchange chromatography using the Dionex CarboPac MA1 anion-exchange column (Thermo Scientific, Dreieich, Germany) with pulsed amperometric detection. Standards of D-glucose, D-xylose, glycerol and ethanol were prepared with deionized water. Supernatants from cell cultures were isolated by centrifugation for 10 minutes at 20.000*g and transfer of the supernatant to a new Eppendorf tube. The analytes in a 10 μl injection were separated using 480 mM sodium hydroxide eluent flowing at 0.4 ml/min for 60 min.

Sequence analysis of the NNmini strain, the NNQ strain and NNQ mutants. For whole genome sequencing, strains were grown overnight in LB medium supplemented with 20 mM glucose and 10 mM acetate. The Macherey-Nagel NucleoSpin Microbial DNA purification Kit (Macherey-Nagel, Duren, Germany) was used to extract the genomic DNA. Microbial short insert PCR-free library construction for single-nucleotide variant detection and generation of 150 bp paired-end reads on an Illumina HiSeq 3000 platform were performed by Novogene (Cambridge, UK). Breseq (Barrick Lab, Texas) (Deatherage & Barrick, 2014) was used to map the obtained reads to the reference genome of E. coli MG1655 (GenBank accession no. U00096.3). Elaboration with the algorithms supplied by the software package allowed to identify single-nucleotide variants (with >50% prevalence in all mapped reads) and regions more than 2 standard coverage deviations from the global median coverage.

Metabolite quantification of strain NNmini ΔldhA +glpD +pIBA. 1 mL of M9 glycerol medium (supplemented with 20 mM acetate or 5 g L⁻¹ CAAs) was inoculated with strain NNmini ΔldhA +glpD +pIBA and incubated directly in a sealed GC headspace glass vial (22.5×46 mm, Chromtech) at 30 or 37° C. and 300 rpm for 3 days. Isobutanol quantification was performed by GC-MS/MS in SIM mode using an Agilent 5975C inert XL EI/CI MSD system (Agilent Technologies) upgraded to MS/MS with an Evolution3 system (Chromtech) and equipped with an HP-5MS Ultra Inert column (dimensions: 30 m, 0.25 mm, 0.25 μm, Agilent Technologies) and a Combi PAL-XT auto sampler (CTC Analytics). The vial was incubated at 80° C. for 3 min and 300 μL of the head space gas were injected with a 500:1 split at 33.9 mL min⁻¹ to the GC-MS/MS system using a 2.5 mL syringe heated to 85° C. The inlet temperature was set to 250° C. and a constant flow of 1 mL min⁻¹ helium was used as carrier gas. The oven temperature was held at 33° C. for 2 min followed by a linear temperature gradient of 25° C. min⁻¹ to a final temperature of 200° C. Mass spectra were recorded starting 1.48 min after injection and m/z values of 55.0, 56.0, and 74.0 were used to detect isobutanol in SIM mode at 6.67 scans s⁻¹. Chromatograms were evaluated with the Agilent ChemStation software (Agilent Technologies). Isobutanol was quantified using a calibration curve prepared with external standards of 1 mL cultivation medium with known isobutanol concentrations measured with the same method as described above. After isobutanol quantification, the vials were opened and the medium was transferred to Eppendorf tubes and centrifuged at 13.000×g for 5 min. Remaining glycerol in the supernatant was quantified using the Liquid Glycerol kit (Enzytec) according to the manufacturers' instructions. Ethanol and acetate were quantified in the supernatant using the Ethanol Assay Kit (Megazyme) and the Acetic Assay Kit (Megazyme), respectively.

### Example 1 Gene Deletion Via P1 Phage Transduction

Gene deletions of putA, dld, mqo, dadA, poxB, kefF, wrbA, fadE, yieF, glpD, gpsA, lldD, lhgO were generated by P1 phage transduction (Thomason et al., Curr Protoc Mol Biol. 2007, Ch. 1:1.17.1-1.17.8). Donor strains were strains JWxxxx from the KEIO collection listed in Table 1 with a kanamycin-resistance gene (KmR). The strains of E. coli with the desired deletions were obtained by genetic recombination with the P1 phage lysate and selected for by plating on kanamycin containing plates. A successful gene deletion was verified by determining the size of the genomic locus by PCR with DreamTaq polymerase (Thermo Scientific, Dreieich, Germany) and the respective KO-Ver primers (Table 2). Furthermore, a PCR with DreamTaq polymerase (Thermo Scientific, Dreieich, Germany) and internal primers (“int”) binding inside of the gene coding sequence was performed to confirm that no copy of the gene to be deleted was present in the genome of the transduced strain. For removal of the selective marker, a fresh culture was grown to OD₆₀₀~0.2, followed by addition of 50 mM L-Rhamnose and cultivating for ~4 h at 30° C. for flippase expression induction. Colonies that only grew on LB medium in absence of the respective antibiotic were isolated and successful removal of the KmR gene from the respective locus was confirmed by PCR using the locus specific KO-Ver primers and with DreamTaq polymerase (Thermo Scientific, Dreieich, Germany).

### Example 2: Gene Deletion by Recombineering

Gene deletion by recombineering involved PCR with “KO” primers (Table 2) with 50 bp homologous overhangs and pKD4 plasmid (Addgene #45605; http://n2t.net/addgene:45605; RRID: Addgene_45605) as template and PrimeStar GXL polymerase (Takara Bio) was performed to generate kanamycin resistance cassettes. E. coli WT cells were prepared for gene deletion by inoculating fresh cultures in LB, followed by induction of the recombinase genes by addition of 15 mM L-arabinose at OD ~0.4-0.5, followed by incubation for 45 min at 37° C. The cells were harvested (11,000 rpm, 30 sec, 2° C.) and washed three times with ice cold 10% glycerol. For electroporation, ~300 ng of Km cassette PCR-product was transformed (1 mm cuvette, 1.8 kV, 25 ρF, 200Ω). Gene deletions were confirmed by selection on kanamycin containing plates and via one PCR using ‘KO-Ver’ primers (Table 2) and one PCR using internal (int) primers (Table 2), both of these being done with DreamTaq polymerase (Thermo Scientific, Dreieich, Germany). The Km cassette was removed by adding 50 mM L-rhamnose to an exponentially growing 2 ml LB culture at OD 0.5 for induction of flippase gene expression. After induction, cells were incubated for ≥3 h at 30° C. After screening colonies for kanamycin sensitivity, removal of antibiotic resistance cassette was confirmed by PCR using ‘KO-Ver’ primers and DreamTaq polymerase (Thermo Scientific, Dreieich, Germany).

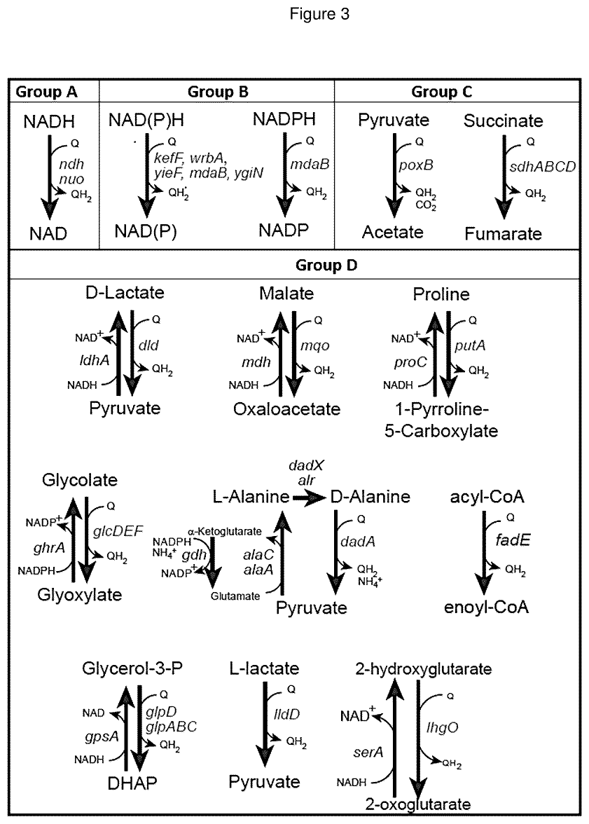

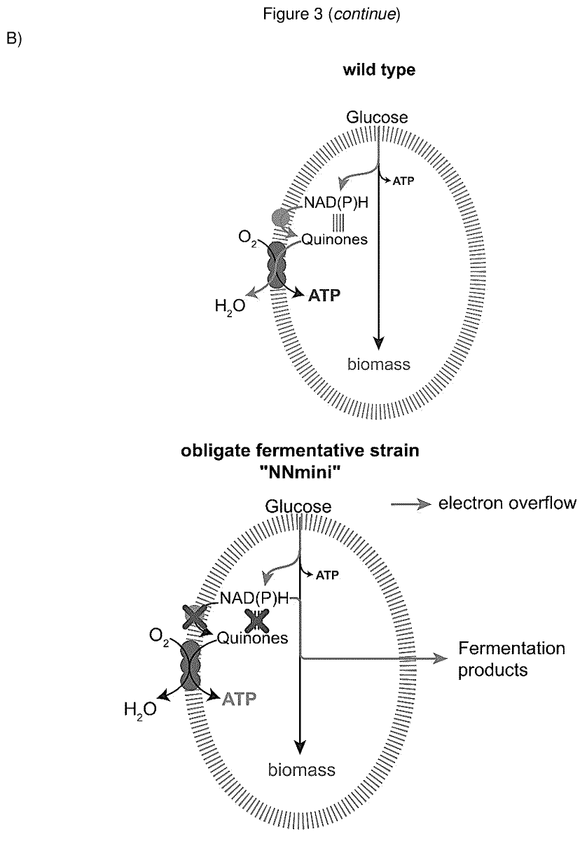

E. coli was deleted of the genes related to two NAD(P)H:quinone oxidoreductases (ndh, nuoEFG, kefF, mdaB-ygiN, wrbA, yieF) and of the genes encoding quinone-dependent dehydrogenases gpsA, sdhABCD, lldD, poxB, fadE, dld, mqo, putA, glpD, glpABC, glcDEF, dadA, lhgO. The latter eight genes could potentially form a “mini-cycle” with an immediate NAD(P)H-dependent counterpart. The resulting genetically engineered bacteria strain was called “NNmini” where “NN” denotes the deletion of the 2 main NADH dehydrogenases and “mini” denotes the elimination of all other quinone-reducing reactions including the mini-cycles (FIG. 3B).

To investigate whether the aerobic fermentative phenotype of the NNmini strain could be achieved with less genetic modifications, the ubiquinone biosynthesis (encoded by ubiCA) was deleted either alone (ΔubiCA strain) or in the ΔnuoEFG Δndh background. The corresponding bacterial strains were genetically engineered. The ΔnuoEFG Δndh ΔubiCA strain was called “NNQ” where “NN” denotes the deletion of the major NADH dehydrogenases and “Q” denotes the deletion of ubiquinone biosynthesis.

### Example 3: Growth and Biomass Production of Genetically Engineered Strains

### NNmini Strain

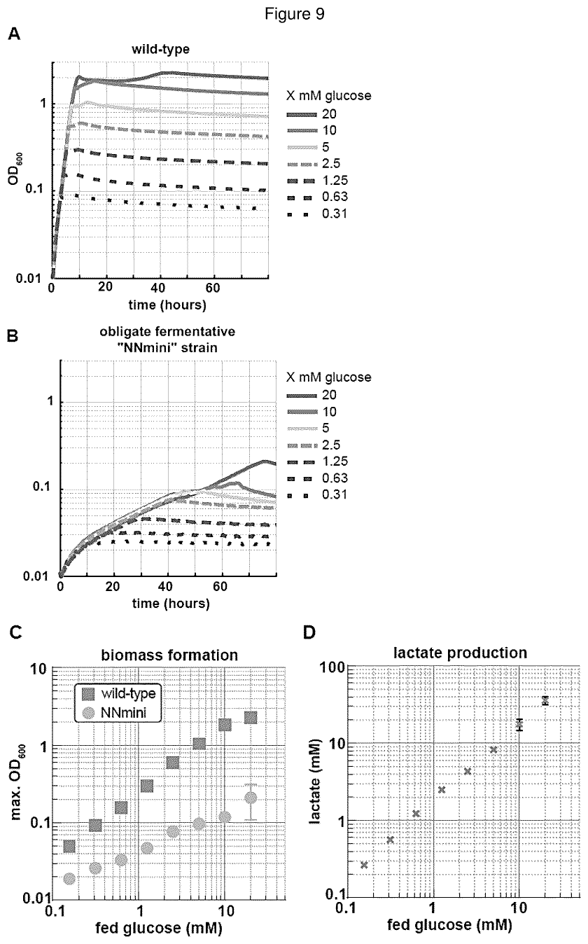

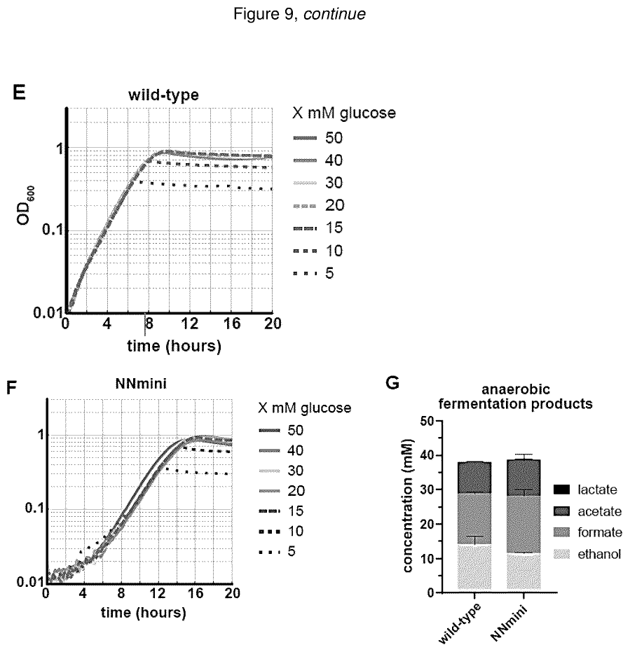

Aerobic growth of a wild-type E. coli strain was compared with that of the engineered NNmini strain on a gradient of glucose concentrations. Here, much slower growth (wildtype growth rate on 20 mM glucose: 0.6502±0.0052 h⁻¹; NNmini growth rate on 20 mM glucose: 0.0465±0.0037 h⁻¹; FIG. 9A, 9B) and lower biomass yields (FIG. 9C) were observed for the NNmini strain, which hinted at a fermentative growth phenotype. To confirm this hypothesis, the inventors quantified metabolites from the culture supernatant and found that the NNmini strain converted fed glucose exclusively to lactate at a yield of 0.9±0.1 g lactate/g while no other fermentation product could be detected (FIG. 9D). This stands in stark contrast to the wild-type strain, where no fermentation product was detected. Thus, the inventors concluded that elimination of quinone reducing reactions and by that electron transfer to the ETC resulted in an obligate fermentative phenotype.

Next, the inventors investigated the anaerobic growth phenotype of both the wild type and of the NNmini strain. For this, the glucose gradient growth experiment was repeated with the NNmini strain and a wild-type control under anaerobic conditions. Interestingly, in anaerobic conditions, growth rates and biomass yields of the NNmini strain were more comparable to the wild-type strain (wildtype growth rate on 20 mM glucose: 0.5095±0.0041 h⁻¹; NNMini growth rate on 20 mM glucose: 0.4185±0.0033 h⁻¹; (FIG. 9E, F). Furthermore, the NNmini strain exhibited higher growth rates and biomass yields than under aerobic conditions (FIG. 9E, F). Concerning production of fermentation products, both strains exhibited hetero-fermentative behavior and excreted acetate, formate and ethanol in comparable amounts (FIG. 9G). Taken together, these findings suggest that the observed aerobic growth of the NNmini strain is less efficient compared to growth under anaerobic conditions, which leads to impaired growth in aerobic conditions.

ΔNdh ΔnuoEFG, ΔubiCA and NNQ Strains

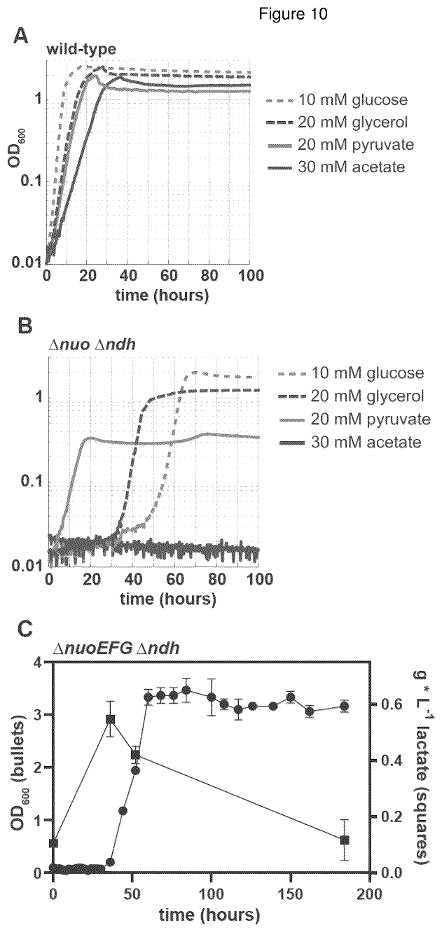

E. coli bacteria with only two gene deletions Δndh ΔnuoEFG, i.e. of the main NADH dehydrogenases, showed an impaired growth phenotype on different carbon sources (FIG. 10A, B) as well as some aerobic lactate production (FIG. 10C). After 50 hours of culture, the Δndh ΔnuoEFG strain grew to high ODs associated with respiratory growth and consumed most of the initially produced lactate, which indicates that it could reactivate cellular respiration.

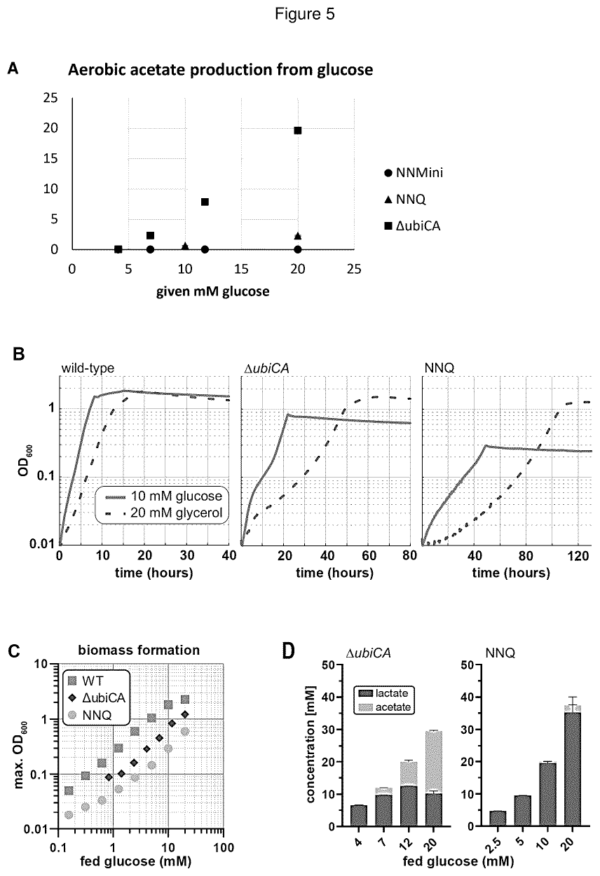

Both the ΔubiCA and NNQ strains showed slower growth rates on glucose and glycerol than observed for the wild type (FIG. 5B).

Biomass yields of the deletion mutants were lower than those obtained for the wild type (FIG. 5C), which hinted at an aerobic fermentative phenotype.

### Example 4: Comparison of Lactate and Acetate Production by the E. coli Engineered Strains Grown on Glucose

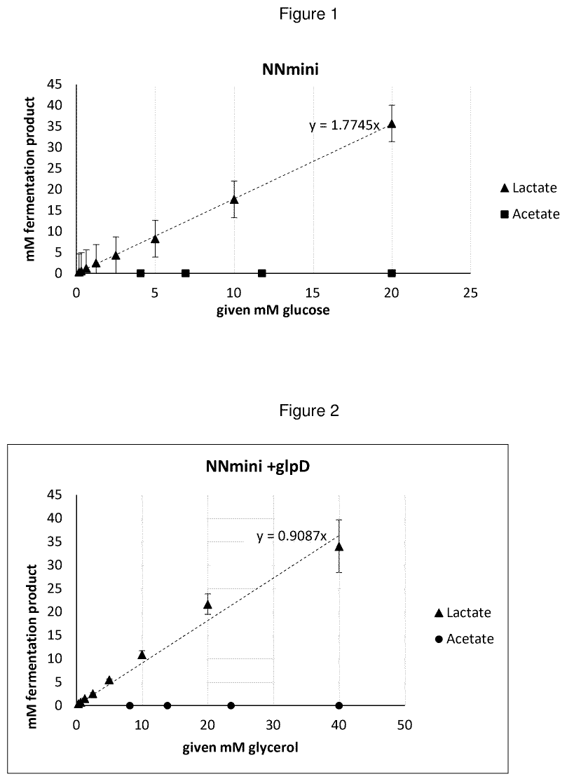

The NNmini strain E. coli was cultivated and grown in minimal medium in presence of increasing concentrations of glucose as explained in the method section. After the cultures reached stationary growth phase, the concentration of the fermentative products lactate and acetate were analyzed in the supernatants of the NNmini strain by Ion Chromatography. Results showed that in the NNmini strain supernatants, lactate concentrations were directly proportional to the glucose concentration in the medium, whereas no acetate could be detected (FIG. 1H, n=3). Thereafter, the production of lactoate and acetate starting from glucose was analysed and compared between the following engineered E. coli strains:
(i) ΔubiCA, deleted in ubiquinone biosynthesis, (ii) NNQ, deleted in both NADH dehydrogenases and ubiquinone biosynthesis,
(iii) NNmini, deleted in both NADH dehydrogenases as well as in reactions which might bypass NADH-dehydrogenase activity, (FIG. 3), and also in glpD gene;
(iv) NNminimal1 strain with Δndh, Δnuo and Δdld deletions

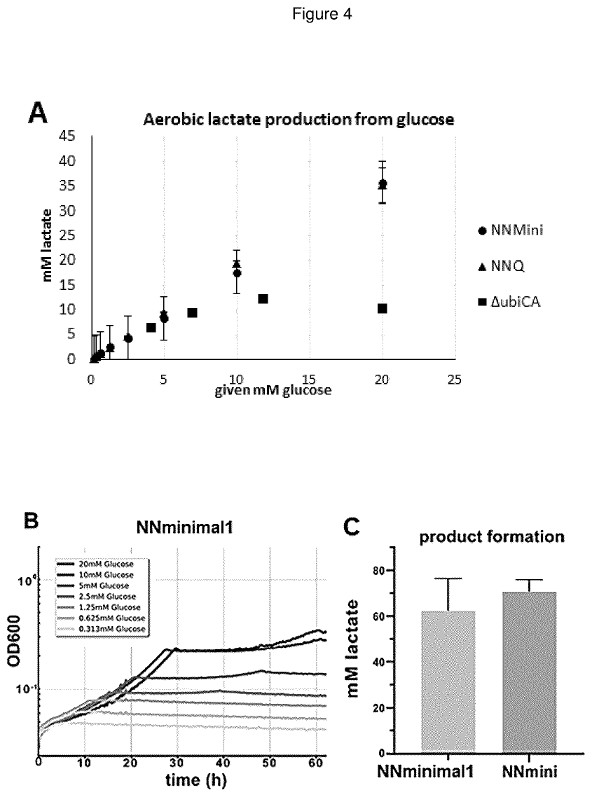

The strains were incubated in M9 minimal medium under ambient (aerobic) conditions in the presence of 20, 10, 5, 2.5, 1.25, 0.6, 0.3, 0.15 mM glucose (NNQ, NNmini, and NNminimal1) or in presence of 20, 11, 6.9, 4.7 mM glucose (ΔubiCA). After the cultures reached stationary growth phase, supernatant samples were taken and analysed by ion-chromatography to determine lactose and acetate concentrations (FIGS. 4 and 5, n=3). The results (FIG. 4) showed that in the supernatants from NNMini(glpD-) and NNQ strains, lactate concentrations were directly proportional to the glucose concentration in the medium, whereas lactate production by ΔubiCA decreased at glucose concentrations higher than 10 mM.

The growth curves of NNminimal1 strains on increasing glucose concentrations was similar to that of the NNmini strain (FIG. 4B). Strain NNminimal1 generated high amounts of lactate also similarly to the NNmini strain (FIG. 4C).

Both ΔubiCA and NNQ strains produced lactate and acetate from glucose (FIG. 5D). Notably, the NNQ strain converted glucose into lactate and only residual acetate at a stoichiometry that is close to the theoretical maximal fermentation product yield for E. coli (FIG. 5D). More in particular, the results (FIG. 5A) showed that in the supernatants from ΔubiCA strain, acetate concentrations were directly proportional to the glucose concentration in the medium. Some acetate production was detected also in the supernatants from NNQ strain, but at niveau much lower than in ΔubiCA supernatant, reaching values of 2.26 mM and 19.6 mM respectively at 20 mM glucose. In contrast, the NNmini strain did not produce any acetate.

As mentioned above, the ability of acetate production under aerobic conditions indicates that NNQ and ΔubiCA strains are somehow able to oxidize pyruvate and transfer some electrons from pyruvate, either directly via PoxB to quinone (other than ubiquinone) or via NADH generated by pyruvate dehydrogenase followed by a leaky activity connecting NADH oxidation and quinone reduction (likely one of the activities/minicycles shown in FIG. 3A). Since experiments were carried out in the presence of oxygen and no formate was detected, NNQ and ΔubiCA strains seem to possess some electron sinks other than pyruvate-formate lyase which allow the secretion of acetate as an unbalanced fermentative product.

### Example 5. Engineering and Analysis of NNmini+GlpD Strain

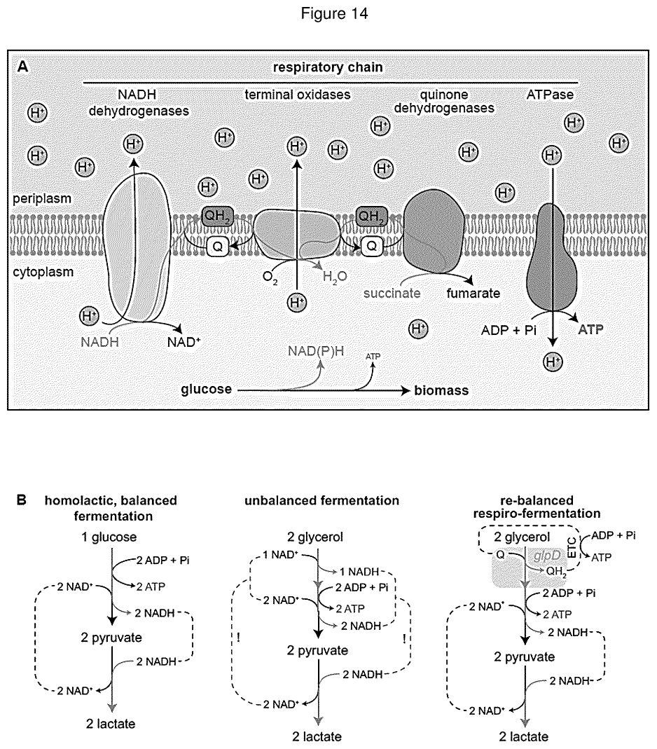

After engineering the obligate fermentative NNmini strain, the inventors aimed to obtain a bacterial strain with unbalanced fermentation and aerobic fermentation of glycerol to lactate (FIG. 14).

Being a byproduct of biodiesel production, glycerol is readily available and considered as a more renewable and sustainable carbon source than glucose. To make glycerol available for fermentative production processes, the NNmini strain was reintegrated of the quinone-dependent glycerol 3-phosphate dehydrogenase (GlpD) for the conversion of glycerol-phosphate to dihydroxyacetone-phosphate, in order to allow maintaining the cellular redox balance while fermenting glycerol to lactate (FIG. 14).

Indeed, since the NNmini strain still harbors an intact ETC, selected quinone-dependent reactions can be reintegrated, so that ubiquinone and oxygen can be used for single oxidation steps. The resulting hybrid mode of respiro-fermentative carbon source utilization with individual respiratory modules is herein referred to as “respiro-fermentation”.

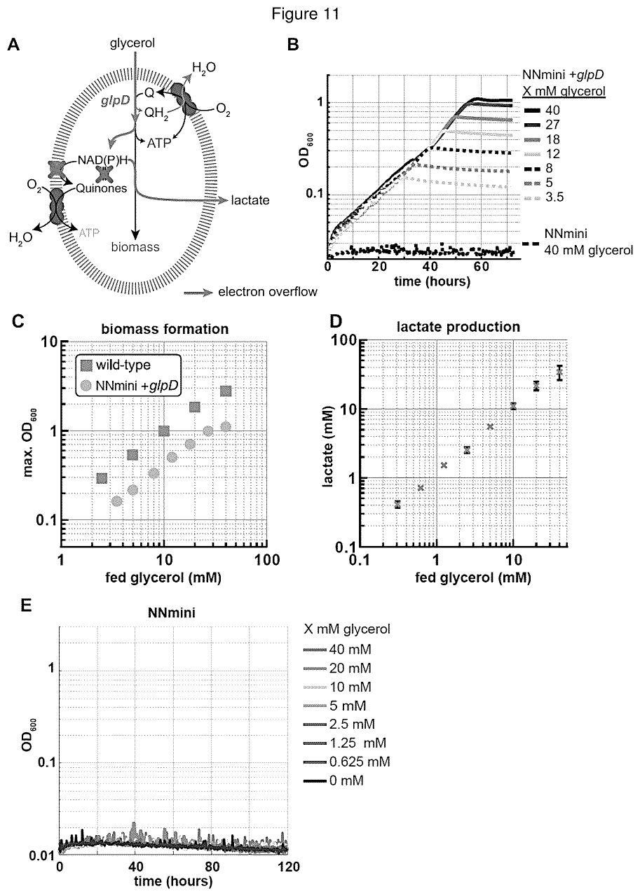

Therefore, NNmini strain was subjected to genomic reintegration of glpD and deleted of the corresponding NADPH-dependent glycerol 3-phosphate dehydrogenase gpsA to avoid the formation of a mini-cycle. While the base NNmini strain was unable to grow on glycerol as sole carbon source (FIG. 11E), NNmini +glpD could aerobically grow on glycerol (growth rate with 40 mM glycerol: 0.0745±0.0019 h⁻¹; FIG. 11B). Interestingly, the observed maximum OD₆₀₀ values were significantly higher compared to the aerobic fermentative growth on glucose (comparing 20 mM glucose with 40 mM glycerol to account for the number of carbon, FIG. 3A and FIG. 11C). This phenotype is likely due to the additional ATPase-based ATP generation, which relies on the GlpD dependent quinol generation to build a proton gradient (FIG. 14).

Next, it was found that the supplied glycerol was converted to lactate by the NNmini +glpD strain in a nearly stoichiometric manner (FIG. 2, FIG. 11D) with acetate as a minor byproduct for high glycerol concentrations (0.04±0.03 mM acetate for 10 mM glycerol, 1.25±0.05 mM acetate for 20 mM glycerol and 1.01±0.92 mM acetate for 40 mM glycerol).

Thereafter, the production of lactate and acetate starting from glycerol was analysed in the following engineered E. coli strains:
(i) ΔubiCA,
(ii) NNQ, and
(iii) NNmini (glpD⁺), with reintroduced chromosomal glycerol 3-phosphate dehydrogenase (glpD).

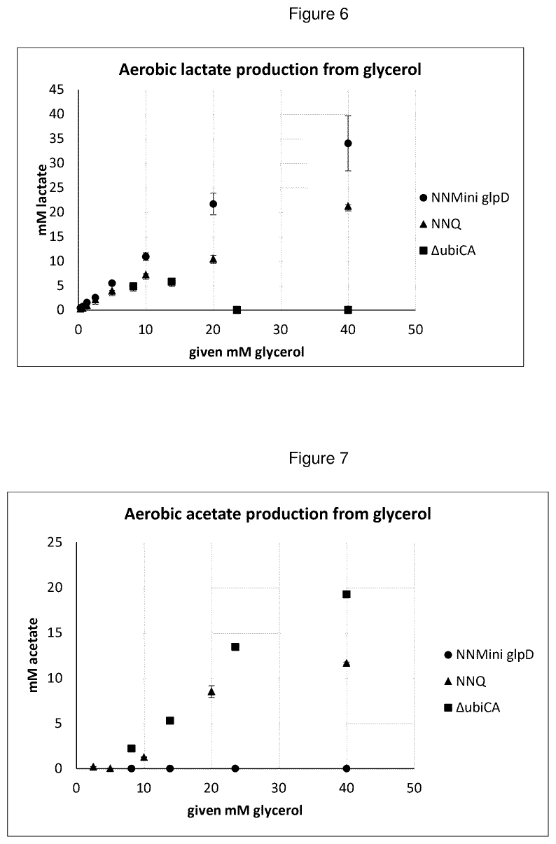

The results indicated an improved lactate production curve by the NNmini +glpD strain, compared to ΔubiCA and NNQ strains (FIG. 6).

Moreover, ΔubiCA and NNQ strains were able to produce acetate from glycerol (FIG. 7), while no acetate was produced by the NNmini glpD⁺ strain. As mentioned above, the ability of acetate production under aerobic conditions indicates that NNQ and ΔubiCA strains are able to transfer some electrons from pyruvate.

### Example 6: Evolution of Engineered Strains

The experiments described in this example were performed to analyse the stability of the phenotypes of the engineered strains.

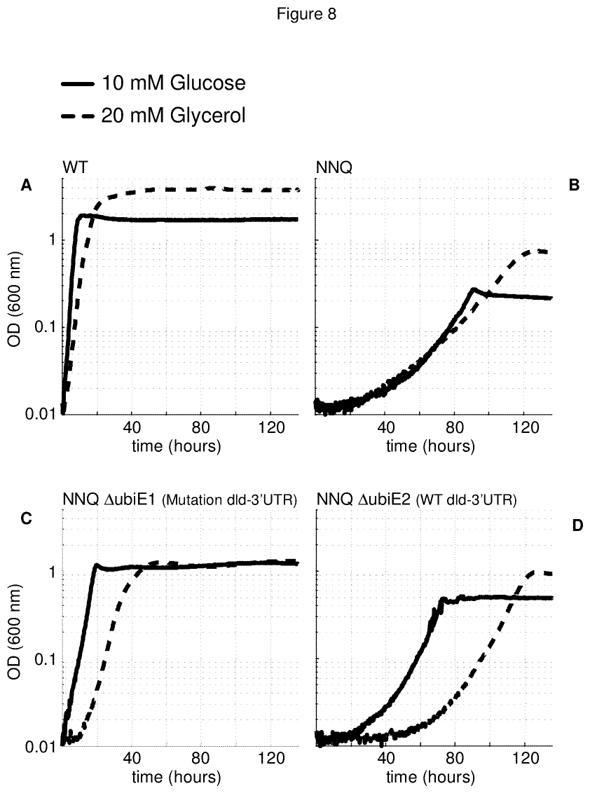

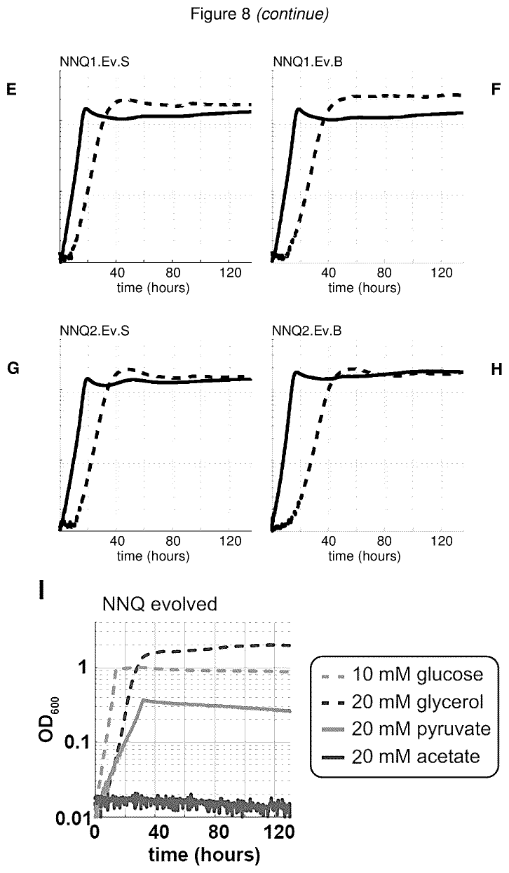

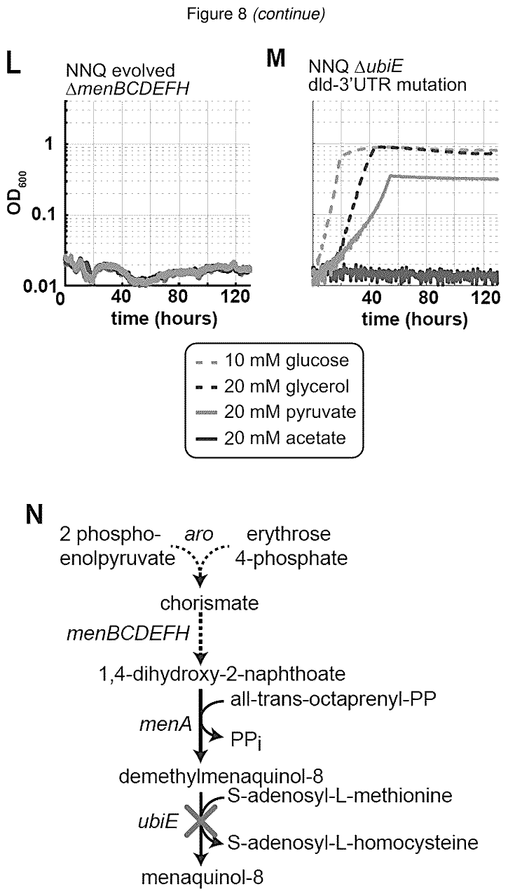

Upon prolonged cultivation (60 days), the NNQ strain grew much faster and to higher cell densities (FIG. 8C, D, G, H, 1) than observed in the previous experiments (FIG. 8A, B), suggesting “evolution” of the NNQ strain, i.e. “evolved NNQ strain”. Genome sequencing of independently selected isolates of “evolved NNQ strains” revealed that they shared mutations in the did promoter region and missense mutations in the ubiE gene. While the did mutation might result in an upregulation of the expression of did and thereby facilitated uptake of previously excreted lactate, the inactivation of UbiE could result in an accumulation of demethylmenaquinone (DMK) (FIG. 8N). DMK accumulation could restore cellular respiration by allowing ubiquinone-dependent dehydrogenases with high K_{M} values for DMK to use it as an electron acceptor.

To verify these hypotheses, the ubiE deletion and the mutation in the 3′-UTR of the did gene were reverse-engineered in a naïve NNQ strain using recombineering, which resulted in an improved growth phenotype (FIG. 8E, F, M). Furthermore, the menaquinone biosynthesis (ΔmenBCDEFH) was deleted in the evolved NNQ strain, which coherently abolished growth on all tested carbon sources (FIG. 8L). The inventors used recombineering to delete the ubiE gene and to introduce the did point mutation. Additionally, IC analysis revealed the absence of fermentative products. Thus, indicating the instability of the fermentative phenotype of the NNQ strain. While a deletion of did might restore the fermentative phenotype, similar events might lead to the activation of other minicycles (FIG. 3).

These results indicate that a simple mutation can circumvent the obligate fermentation phenotype in the NNQ strain.

In none of the experiments the NNmini strain ever circumvented the selection, probably since there all quinone dependent dehydrogenases were deleted. Therefore, this characteristic seems due to the combination of the additional deletions of the genes poxB, sdhABCD, glcDEF, lhgO, putA, dld, lldD, dadA, kefF, mdaB-ygiN, glpABC, glpD, mqo, gpsA, wrbA, yieF, and fadE.

Taken together, these findings demonstrate that the NNQ strain exhibited only an unstable aerobic fermentative phenotype. This limits biotechnological applications especially for long time cultivations. Furthermore, it is not possible to use oxygen for single quinone-dependent reactions like in the NNmini strain, which does not allow controlled unbalanced fermentations.

### Example 7: Acetate, Pyruvate or Casamino Acid Supplementation Improves Aerobic Biomass Yields of the Obligate Fermentative Strain

To some extent, aerobic fermentative growth of the NNmini strain was expected to be worse than in anaerobic conditions since natively the switch from respiration to fermentation is governed by the oxygen mediated expression control of ~200 genes though key regulators like the ArcAB system and FNR. However, the metabolic engineering approach followed for the NNmini strains disclosed herein neglected such regulation. Therefore, it could be that unnecessary respiratory genes might be expressed while needed fermentative genes might be repressed during fermentative growth of the NNmini strain in aerobic conditions. This leads to increased metabolic burden and thus worse growth performance. While these regulatory factors might play a role, whole-genome sequencing of the NNmini strain showed that there was any mutation concerning the known regulators of the fermentative or respiratory metabolism.

Therefore, the ability to secrete acetate and ethanol thereby increasing the glycolytic ATP yield could explain the improved biomass yields and growth rates of the NNmini strain in anaerobic conditions.

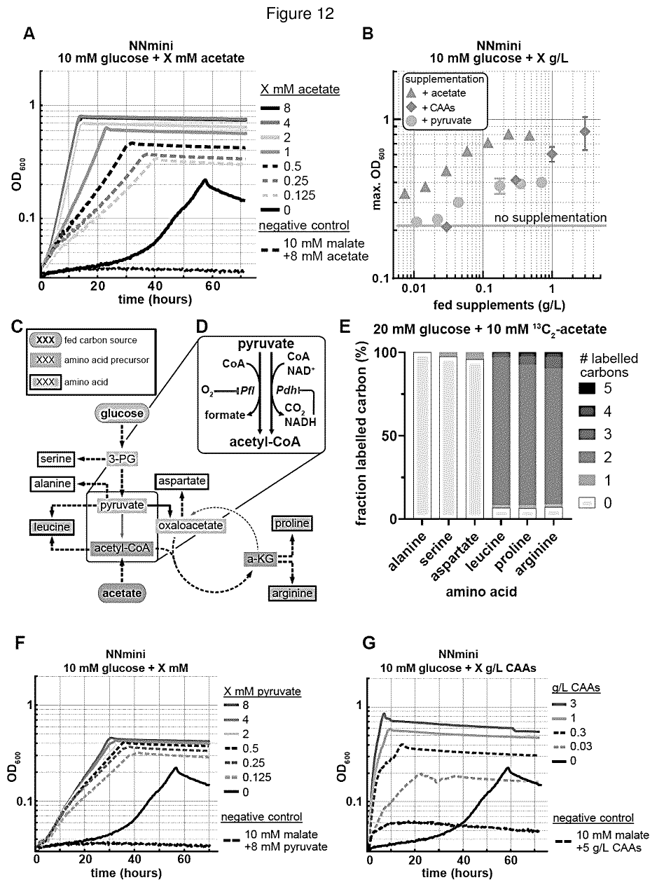

In contrast, the absence of pyruvate-formate lyase (Pfl) activity in aerobic conditions might impair growth of the NNmini strain. Since Pfl uses radical chemistry to catalyze the non-oxidative cleavage of pyruvate to acetyl-CoA and formate, the enzyme is oxygen sensitive. Due to the absence of pyruvate-formate lyase activity, the only remaining way of generating acetyl-CoA and all downstream metabolites in aerobic conditions is via the NADH-producing pyruvate dehydrogenase (Pdh) (FIG. 12D). This has several implications: the NADH production from Pdh interferes with the redox-balanced conversion of glucose to lactate. While under anaerobic conditions, the main alcohol dehydrogenase AdhE of E. coli could alleviate this redox imbalance through ethanol production as an electron sink, it is not expressed under aerobic conditions. Additionally, Pdh is inhibited by high NADH concentrations and might therefore produce acetyl-CoA inefficiently (FIG. 12D). This abolishes the possibility to produce and excrete acetate to generate additional molecule of ATP per pyruvate. Since its production does not consume NADH, under anaerobic conditions, E. coli achieves redox-balancing via mixed acid fermentation (i.e. excretion of ethanol).

Taken together, these facts suggest that the impaired aerobic growth phenotype of the NNmini strain stems from a slight acetyl-CoA auxotrophy. In the following, the inventors aimed to test whether the supplementation of biomass precursors from lower metabolism could relieve this auxotrophy.

Therefore, the NNmini strain was grown on 10 mM glucose supplemented with different concentrations of acetate, pyruvate or casamino acids (CAAs). All three supplements improved both the growth rate and the formation of biomass to a varying extent (FIG. 12 A, B, F, G). While growth improvements from supplementation with acetate reached a plateau after 5 mM addition (maximum OD₆₀₀=0.6), CAA supplementation resulted in maximum OD₆₀₀ values similar to a wild type growing on 10 mM glucose (OD₆₀₀>1). CAAs are obtained from acid hydrolysis of casein and contain all amino acids except tryptophan. Their supplementation therefore yields free amino acids from lower metabolism. This lowers the dependency of the NNmini strain on acetyl-CoA generation via the inhibited Pdh, which explains the increase in biomass yield. In the case of pyruvate supplementation, the NNmini biomass yields were lower than with acetate. Indeed, converting the supplied pyruvate to acetyl-CoA still results in the formation of one NADH. Therefore, 1 molecule of pyruvate needs to be converted to lactate (−1 NADH) to allow the Pdh-catalyzed conversion of pyruvate to acetyl-CoA (+1 NADH), which explains the lower biomass yields with pyruvate supplementation compared to growth with acetate. These findings are in line with the speculated acetyl-CoA auxotrophy of the NNmini strain resulting from NADH-inhibited Pdh.

Industrially, compared to pyruvate or CAAs, acetate is the best-suited supplement as it is cheap and can be produced from industrial waste streams. Therefore, the NNmini strain was grown on ¹³C₂-acetate and analyzed for the isotopic labelling patterns in selected proteinogenic amino acids (FIG. 12C, E). The labeled carbon was traced throughout metabolism looking at alanine as a pyruvate-derived amino acid, serine from 3-phosphoglycerate and aspartate from oxaloacetate as example amino acids from upper metabolism precursors. Proline and arginine that are derived from α-ketoglutarate as well as leucine originating from pyruvate and acetyl-CoA were predicted to contain labelled carbons from fed ¹³C₂-acetate. Indeed, the analysis results indicated that labelled carbon from acetate was only incorporated in leucine, proline and arginine (FIG. 12E). Thus, acetyl-CoA is indeed made from the supplied acetate and further incorporated into TCA cycle intermediates until α-ketoglutarate. From there, the deletions of sdhCDAB and mqo prevent further acetate utilization via the glyoxylate shunt, which is reflected in the absence of labelled carbon in alanine, serine and aspartate. Therefore, while the majority of cellular biomass is still made from the supplied glucose, using acetate for acetyl-CoA synthesis appears to be beneficial for the strain and could be considered in fermentation processes where high microbial biomasses are required.

### Example 8: Respiro-Fermentative Isobutanol Production from Glycerol by the NNmini glpD⁺ pIBA⁺ Strain

Isobutanol production has been engineered and optimized in E. coli as well as bulk production hosts like Corynebacterium glutamicum and Saccharomyces cerevisiae.

Here, the formation of native fermentation products has been abolished, which enforced the growth-coupled production of isobutanol as sole electron sink for redox balance maintenance. However, all of these studies only focused on the balanced fermentation of isobutanol from glucose. Notably, fermentative isobutanol production from glycerol was demonstrated in Klebsiella pneumoniae (Oh, B. R. et al. Production of 2-butanol from crude glycerol by a genetically-engineered Klebsiella pneumoniae strain. Biotechnol. Lett. 36, 57-62 (2014)). However, the production was not achieved via growth-coupling with isobutanol as the only fermentation product since the engineered K. pneumoniae strain relied on its native 1,3-propanediol production for redox balance maintenance.

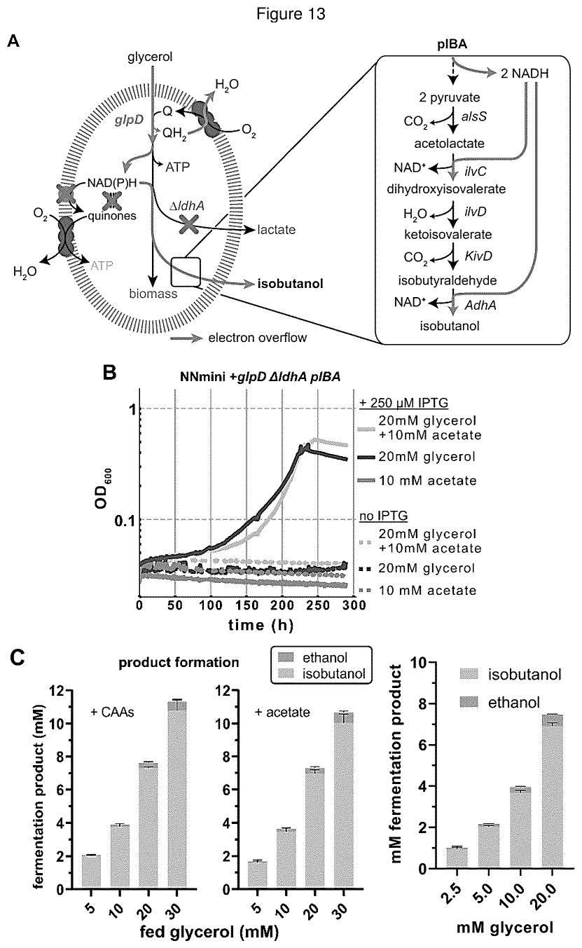

The inventors aimed to use the engineered NNmini strain for the re-balanced, growth-coupled fermentation of glycerol to isobutanol. To redirect the entire fermentative metabolism of the NNmini +glpD strain from lactate as main fermentation product to isobutanol, the NNmini strain was transformed with an isobutanol production plasmid (pIBA4, further on referred to as pIBA, previously described by Ghosh et al, 2019) and deleted of IdhA. The plasmid pIBA4 comprises the genes encoding the enzymes catalizing the reactions to convert piruvate to isobutanol, and namely AlsS, IlvC, IlvD, KivD and AdhA (FIG. 13A). Stoichiometrically, the production of isobutanol should be able to replace the native lactate fermentation while maintaining the cellular redox balance. Subsequently, the inventors demonstrated that the NNmini ΔldhA +glpD +pIBA could grow with glycerol as sole carbon and energy source (FIG. 13B). Since the genes responsible for isobutanol production are under the control of an IPTG-inducible promoter on the pIBA plasmid, a condition without IPTG served as a negative control. While growth on glycerol could be achieved, the strain exhibited a long lag phase. The inventors reasoned that this could potentially be attributed to the associated protein and plasmid burden as the plasmid was optimized for a different genetic background. Next, analysis of metabolites from the culture supernatants showed that glycerol was indeed converted to isobutanol (FIG. 13C). While the observed titers are more than double the yield reported for isobutanol production from glycerol in K. pneumoniae, these yields are still lower than stoichiometrically expected: in theory, a maximum of 10 mM isobutanol could be produced from 20 mM glycerol (FIG. 13C). Besides isobutanol, some ethanol was also detected in the growth medium. This might be due to the overexpression of adhA, which is part of the isobutanol biosynthesis pathway but has an ethanol producing side activity with acetaldehyde. Notably, the isobutanol yields achieved with the NNmini ΔldhA +glpD +pIBA could potentially be negatively affected by the evaporation of the highly volatile isobutanol from the growth medium, which has previously been identified as problem in microbial isobutanol production. Nonetheless, our findings demonstrate for the first time the respiro-fermentative production of isobutanol from glycerol. Furthermore, this is the first demonstration of aerobic growth-coupled production of isobutanol. This showcases that the concept of the NNmini strain frees fermentation from the limitation of requiring strictly redox-balanced substrate/product combinations. In the future, performing enzyme expression optimization as it was carried out in the original paper presenting the pIBA plasmid or simply adaptive laboratory evolution could be used to improve growth and isobutanol production.

TABLE 1
E. coli strains and plasmids used in this study

| | | |
|---|---|---|
| Name | Description and genotype | Source |

Strains

| | | |
|---|---|---|
| DH5α | Cloning of overexpression constructs | |
| E. coli SIJ_488 | E. coli K-12 MG1655 Tn7::para-exo-beta-gam; prha-FLP; | Jensen et al. |
| (WT) | xylSpm-Iscel | 2015. Sci Rep. |
| | | 5: 17874. |
| ΔubiCA | ΔubiCA | This study |
| NNQ | Δndh ΔnuoEFG ΔubiCA | This study |
| NNMini | Δndh ΔnuoEFG Δdld Δmqo ΔpoxB ΔsdhABCD ΔglcDEF | This study |
| | ΔlhgO ΔputA ΔlldD ΔdadA ΔkefF ΔmdaB-ygiN ΔglpABC | |
| | ΔwrbA ΔfadE ΔyieF ΔyieF Δ fadE ΔglpD | |
| NNMini + glpD | Δndh ΔnuoEFG Δdld Δmqo ΔpoxB ΔsdhABCD ΔglcDEF | This study |
| | ΔlhgO ΔputA ΔlldD ΔdadA ΔkefF ΔmdaB-ygiN ΔglpABC | |
| | ΔwrbA Δfad E ΔyieF AgpsA | |
| NNMinimal1 | Δndh ΔnuoEFG Δdld | This study |
| NNMinimal2 | Δndh ΔnuoEFG Δdld Δmqo | This study |
| NNMini + glpD | NNMini + glpD deleted in ldhA and expressing isobutanol | This study |
| ΔldhA + plBA | production pathway from plBA plasmid | |
| JW0999 | ΔputA donor: | Keio collection |
| | F−, Δ(araD-araB)567, ΔlacZ4787(::rrnB-3), λ⁻, | |
| | ΔputA758::kan, rph-1, Δ(rhaD-rhaB)568, hsdR514 | |
| JW2121 | Δdld donor: | Keio collection |
| | F−, Δ(araD-araB)567, ΔlacZ4787(::rrnB-3), λ⁻, Δdld- | |
| | 759::kan, rph-1, Δ(rhaD-rhaB)568, hsdR514 | |
| JW2198 | Δmqo donor: | Keio collection |
| | F−, Δ(araD-araB)567, ΔlacZ4787(::rrnB-3), λ−, Δmqo- | |
| | 763::kan, rph-1, Δ(rhaD-rhaB)568, hsdR514 | |
| JW1178 | ΔdadA donor: | |
| | F−, Δ(araD-araB)567, ΔlacZ4787(::rrnB-3), λ−, | |
| | ΔdadA778::kan, rph-1, Δ(rhaD-rhaB)568, hsdR514 | |
| JW0855 | ΔpoxB donor: | Keio collection |
| | F−, Δ(araD-araB)567, ΔlacZ4787(::rrnB-3), λ−, | |
| | ΔpoxB772::kan, rph-1, Δ(rhaD-rhaB)568, hsdR514 | |
| JW0045 | ΔkefF donor:: | Keio collection |
| | F−, ΔkefF759::kan, Δ(araD-araB)567, ΔlacZ4787(::rrnB-3), | |
| | λ−, rph-1, Δ(rhaD-rhaB)568, hsdR514 | |
| JW0989 | ΔwrbA donor:: | Keio collection |
| | F−, Δ(araD-araB)567, ΔlacZ4787(::rrnB-3), λ−, | |
| | ΔwrbA748::kan, rph-1, Δ(rhaD-rhaB)568, hsdR514 | |
| JW5020 | ΔfadE donor:: | Keio collection |
| | F−, Δ(araD-araB)567, ΔfadE739::kan, ΔlacZ4787(::rrnB-3), | |
| | λ−, rph-1, Δ(rhaD-rhaB)568, hsdR514 | |
| JW3691 | ΔyieF donor:: | Keio collection |
| | F−, Δ(araD-araB)567, ΔlacZ4787(::rrnB-3), λ−, rph-1, | |
| | ΔyieF744::kan, Δ(rhaD-rhaB)568, hsdR514 | |
| JW3389 | ΔglpD donor:: | Keio collection |
| | F−, Δ(araD-araB)567, ΔlacZ4787(::rrnB-3), λ−, | |
| | ΔglpD759::kan, rph-1, Δ(rhaD-rhaB)568, hsdR514 | |
| JW3580 | ΔlldD donor:: | Keio collection |
| | F−, Δ(araD-araB)567, ΔlacZ4787(::rrnB-3), λ−, | |
| | ΔlldD789::kan, rph-1, Δ(rhaD-rhaB)568, hsdR514 | |
| JW2635 | ΔlhgO donor:: | Keio collection |
| | F−, Δ(araD-araB)567, ΔlacZ4787(::rrnB-3), λ−, | |
| | ΔygaF741::kan, rph-1, Δ(rhaD-rhaB)568, hsdR514 | |

Plasmids

| | | |
|---|---|---|
| pIBA4 | 814bb-P_{T7A1-O34}-RBS_{AS7}-alsS_{Bsu}-RBS_{AC7}-ilVC_{Eco-6E6}-RBS_{AD5}- | Ghosh et al. |
| | ilvD_{Eco}-RBS_{AK7}-kivd_{Lla}-RBS_{AA2}-adhA_{Lla-M5} -gfp-T_{P22} | 2019, Metabolic |
| | | Engineering 52: |
| | | 324-340 |

Information on genotypes of strains from Keio collection can be found at https://cgsc.biology.yale.edu/StrainQueryForm.php

TABLE 2
Oligonucleotide primers used (Sequences in Sequence Protocol)
‘KO’ primers were used to amplify the Km knockout
cassette from pKD4 with 50 bp gene-specific upstream and
downstream sequences. To verify gene replacement by
kanamycin resistance cassette and cassette removal
by flippase, ‘KO-Ver’-primers (knockout-
verification) were used. Internal primers were
used to verify successful removal of the gene
from the genome.

| SEQ ID | name | purpose |
|---|---|---|
| NO 1 | Ndh_KO_fwd | Cassette construction |
| NO 2 | Ndh_KO_rvs | Cassette construction |
| NO 3 | Ndh_KO_Ver_fwd | Deletion verification |
| NO 4 | Ndh_KO_Ver_rvs | Deletion verification |
| NO 5 | Ndh_int_fwd | Deletion verification |
| NO 6 | Ndh_int_rvs | Deletion verification |
| NO 7 | NuoEFG_KO_fwd | Cassette construction |
| NO 8 | NuoEFG_KO_rvs | Cassette construction |
| NO 9 | NuoEFG_KO_Ver_fwd | Deletion verification |
| NO 10 | NuoEFG_KO_Ver_rvs | Deletion verification |
| NO 11 | NuoEFG_int_fwd | Deletion verification |
| NO 12 | NuoEFG_int_rvs | Deletion verification |
| NO 13 | ubiCA_KO_fwd | Cassette construction |
| NO 14 | ubiCA_KO_rvs | Cassette construction |
| NO 15 | ubiCA_KO_Ver_fwd | Deletion verification |
| NO 16 | ubiCA_KO_Ver_rvs | Deletion verification |
| NO 17 | putA_KO_Ver_fwd | Deletion verification |
| NO 18 | putA_KO_Ver_rvs | Deletion verification |
| NO 19 | putA_int_fwd | Deletion verification |
| NO 20 | putA_int_rvs | Deletion verification |
| NO 21 | glcDEF_KO_fwd | Cassette construction |
| NO 22 | glcDEF_KO_rvs | Cassette construction |
| NO 23 | glcDEF_KO_Ver_fwd | Deletion verification |
| NO 24 | glcDEF_KO_Ver_rvs | Deletion verification |
| NO 25 | glcDEF_int_fwd | Deletion verification |
| NO 26 | glcDEF_int_rvs | Deletion verification |
| NO 27 | dld_KO_Ver_fwd | Deletion verification |
| NO 28 | dld_KO_Ver_rvs | Deletion verification |
| NO 29 | dld_int_fwd | Deletion verification |
| NO 30 | dld_int_rvs | Deletion verification |
| NO 31 | mqo_KO_Ver_fwd | Deletion verification |
| NO 32 | mqo_KO_Ver_rvs | Deletion verification |
| NO 33 | mqo_int_fwd | Deletion verification |
| NO 34 | mqo_int_rvs | Deletion verification |
| NO 35 | dadA_KO_Ver_fwd | Deletion verification |
| NO 36 | dadA_KO_Ver_rvs | Deletion verification |
| NO 37 | dadA_int_fwd | Deletion verification |
| NO 38 | dadA_int_rvs | Deletion verification |
| NO 39 | poxB_KO_Ver_fwd | Deletion verification |
| NO 40 | poxB_KO_Ver_rvs | Deletion verification |
| NO 41 | poxB_int_fwd | Deletion verification |
| NO 42 | poxB_int_rvs | Deletion verification |
| NO 43 | kefF_KO_Ver_fwd | Deletion verification |
| NO 44 | kefF_KO_Ver_rvs | Deletion verification |
| NO 45 | kefF_int_fwd | Deletion verification |
| NO 46 | kefF_int_rvs | Deletion verification |
| NO 47 | wrbA_KO_Ver_fwd | Deletion verification |
| NO 48 | wrbA_KO_Ver_rvs | Deletion verification |
| NO 49 | wrbA_int_fwd | Deletion verification |
| NO 50 | wrbA_int_rvs | Deletion verification |
| NO 51 | fadE_KO_Ver_fwd | Deletion verification |
| NO 52 | fadE_KO_Ver_rvs | Deletion verification |
| NO 53 | fadE_int_fwd | Deletion verification |
| NO 54 | fadE_int_rvs | Deletion verification |
| NO 55 | yieF_KO_Ver_fwd | Deletion verification |
| NO 56 | yieF_KO_Ver_rvs | Deletion verification |
| NO 57 | yieF_int_fwd | Deletion verification |
| NO 58 | yieF_int_rvs | Deletion verification |
| NO 59 | mdaB-ygiN_KO_C_R | Cassette construction |
| NO 60 | mdaB-ygiN_KO_V_F | Cassette construction |
| NO 61 | mdaB-ygiN_KO_Ver_fwd | Deletion verification |
| NO 62 | mdaB-ygiN_KO_Ver_rvs | Deletion verification |
| NO 63 | mdaB-ygiN_int_fwd | Deletion verification |
| NO 64 | mdaB-ygiN_int_rvs | Deletion verification |
| NO 65 | glpD_KO_Ver_fwd | Deletion verification |
| NO 66 | glpD_KO_Ver_rvs | Deletion verification |
| NO 67 | glpD_int_fwd | Deletion verification |
| NO 68 | glpD_int_rvs | Deletion verification |
| NO 69 | lldD_KO_Ver_fwd | Deletion verification |
| NO 70 | lldD_KO_Ver_rvs | Deletion verification |
| NO 71 | lldD_int_fwd | Deletion verification |
| NO 72 | lldD_int_rvs | Deletion verification |
| NO 73 | glpABC_KO_C_F | Cassette construction |
| NO 74 | glpABC_KO_C_R | Cassette construction |
| NO 75 | glpABC_KO_Ver_fwd | Deletion verification |
| NO 76 | glpABC_KO_Ver_rvs | Deletion verification |
| NO 77 | glpABC_int_fwd | Deletion verification |
| NO 78 | glpABC_int_rvs | Deletion verification |
| NO 79 | sdhABCD_KO_C_F | Cassette construction |
| NO 80 | sdhABCD_KO_C_R | Cassette construction |
| NO 81 | sdhABCD_KO_Ver_fwd | Deletion verification |
| NO 82 | sdhABCD_KO_Ver_rvs | Deletion verification |
| NO 83 | sdhABCD_int_fwd | Deletion verification |
| NO 84 | sdhABCD_int_rvs | Deletion verification |
| NO 85 | lhgO_KO_Ver_fwd | Deletion verification |
| NO 86 | lhgO_KO_Ver_rvs | Deletion verification |
| NO 87 | lhgO_int_fwd | Deletion verification |
| NO 88 | lhgO_int_rvs | Deletion verification |
| NO 89 | gpsA_KO_F | Cassette construction |
| NO 90 | gpsA_KO_R | Cassette construction |
| NO 91 | gpsA_KO_V_F | Deletion verification |
| NO 92 | gpsA_KO_V_R | Deletion verification |
| NO 93 | gpsA fwd internal | Deletion verification |
| NO 94 | gpsA internal Rev | Deletion verification |
| NO 95 | ldhA_KO_F | Cassette construction |
| NO 96 | ldhA_KO_R | Cassette construction |
| NO 97 | ldhA_KO_Ver_fwd | Deletion verification |
| NO 98 | ldhA_KO_Ver_rvs | Deletion verification |
| NO 99 | ldhA_int_fwd | Deletion verification |
| NO 100 | ldhA_int_rvs | Deletion verification |

TABLE 3
List of gene deletions to obtains the genetically engineered bacteria

| Gr. | Abbreviation | Encoded protein | Gene locus |
|---|---|---|---|
| D | dadA | D-amino acid dehydrogenase | b1189 |
| D | dld | quinone-dependent D-lactate | b2133 |
| | | dehydrogenase | |
| D | fadE | Acyl-coenzyme A dehydrogenase | b0221 |
| D | glcDEF, or glcD, | glycolate oxidase subunits | D: b2979; E: b4468; |
| | glcE, glcF | D, E, and F | F: b4467, |
| D | glpABC or glpA, | Anaerobic glycerol-3-phosphate | A: b2241; B: b2242; |
| | glpB, glpC | dehydrogenase subunit A, B, C | C: b2243, |
| D | glpD | glycerol-3-phosphate dehydrogenase | b3426 |
| D | gpsA | Glycerol-3-phosphate dehydrogenase | b3608 |
| | | [NAD(P)⁺] (GPDA) | |
| B | kefF | regulator of KefC-mediated | b0046 |
| | | potassium transport and quinone | |
| | | oxidoreductase | |
| D | lhgO | L-2-hydroxyglutarate dehydrogenase | b2660 |
| D | lldD | L-lactate dehydrogenase | b3605 |
| B | mdaB | NADPH:quinone oxidoreductase MdaB | b3028 |
| D | mqo | malate:quinone oxidoreductase | b2210 |
| A | ndh | enzyme NADH:quinone | b1109 |
| | | oxidoreductase II | |
| A | nuoABCDEFGHIJKLMN | encode the 13-14 subunits of the | A: b2288, B: b2287, |
| | or | protein complex NADH:ubiquinone | CD: b2286, E: b2285, |
| | nuoA, nuoB, nuoCD, | oxidoreductase I, NDH-1: A, B, C/D, | F: b2284, G: b2283, |
| | nuoE, nuoF, | E, F, G, H, I, J, K, L, M | H: b2282, I: b2281, |
| | nuoG, nuoH, nuoI, | | J: b2280, K: b2279, |
| | nuoJ, nuoK, nuoL, | | L: b2278, M: b2277 |
| | nuoM | | |
| C | poxB | Pyruvate dehydrogenase | b0871 |
| | | [ubiquinone] | |
| D | putA | used DNA-binding transcriptional | b1014 |
| | | repressor/proline dehydrogenase/ | |
| | | 1-pyrroline-5-carboxylate | |
| | | dehydrogenase PutA | |
| C | sdhABCD or sdhA, | Succinate dehydrogenase | A: b0723; B: b0724; |
| | sdhB, sdhC, sdhD | flavoprotein subunit A, B, C, or D | C: b0721; D: b0722 |
| B | wrbA | NAD(P)H:quinone oxidoreductase | b1004 |
| B | ygiN | quinol monooxygenase YgiN | b3029 |
| B | yieF | quinone reductase | b3713 |
| | ldhA | enzyme D-lactate dehydrogenase | b1380 |

Group A: NDH activity, group B: described NDH activity, group C: quinone reducing reactions from central metabolism; group D: NDH bypassing activities.

What is claimed is:

1. A genetically engineered bacterium comprising:
a deletion of ndh gene;
a deletion of one or more nuo genes;
a deletion of dld gene,
wherein the one or more nuo genes are selected from nuoA gene, nuoB gene, nuoC gene, nuoD gene, nuoE gene, nuoF gene, nuoG gene, nuoH gene, nuoI gene, nuoJ gene, nuoK gene, nuoL gene, nuoM gene and nuoN gene;
wherein the genetically engineered bacterium is able to use oxygen as electron acceptor,
wherein the bacterium is genetically engineered to produce a fermentation product in presence of a terminal electron acceptor, preferably in the presence of oxygen.

2. The genetically engineered bacterium according to claim 1 further comprising a deletion of mqo gene.

3. The genetically engineered bacterium according to claim 1 further comprising a deletion of gpsA gene.

4. The genetically engineered bacterium according to claim 1 further comprising a deletion of one or more genes selected from the group comprising or consisting of:
poxB gene, sdhABCD genes, glcDEF genes, lhgO gene, putA gene, lldD gene, dadA gene, kefF gene, mdaB-ygiN genes, glpABC genes, wrbA gene, yieF gene, and fadE gene.

5. The genetically engineered bacterium according to claim 1, wherein the genetically engineered bacterium is derived from a facultative anaerobic bacterium selected from the group comprising Enterobacteriaceae, Staphylococcaceae, Streptococcaceae, Lactobacillaceae, Vibrionaceae, and Pasteurellaceae.

6. The genetically engineered bacterium according to claim 1, wherein the genetically engineered bacterium is an E. coli.

7. The genetically engineered bacterium according to claim 1, wherein the genetically engineered bacterium comprises a deletion of ndh gene, nuoEFG genes, and dld gene.

8. The genetically engineered bacterium according to claim 1, wherein the genetically engineered bacterium comprises a deletion of ndh gene, nuoEFG genes, dld gene, mqo gene, gpsA gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene.

9. The genetically engineered bacterium according to claim 1, wherein the genetically engineered bacterium does not comprise a deletion of glpD gene.

10. The genetically engineered bacterium according to claim 1, wherein the bacterium produces D-lactate from a carbon source in presence of oxygen.

11. The genetically engineered bacterium according to claim 10, wherein the carbon source is selected from the group comprising or consisting of glucose, fructose, xylose, arabinose, galactose, mannose, sucrose, cellobiose, hemicellulose, and glycerol or a combination thereof.

12. The genetically engineered bacterium according to claim 10, wherein the carbon source is selected from glucose and glycerol or a combination thereof.

13. (canceled)

14. The genetically engineered bacterium according to claim 8, wherein the genetically engineered bacterium further comprises a deletion of ldhA gene, and
wherein the genetically engineered bacterium further comprises an engineered metabolic pathway for converting pyruvate to isobutanol, wherein said engineered metabolic pathway comprises AlsS, IlvC, IlvD, KivD and SdhA.

15. The genetically engineered bacterium according to claim 14, wherein the bacterium produces isobutanol and/or ethanol, preferably isobutanol from a carbon source in presence of oxygen.

16. The genetically engineered bacterium according to claim 15, wherein the carbon source is selected from the group comprising or consisting of glucose, fructose, xylose, arabinose, galactose, mannose, sucrose, cellobiose, hemicellulose, and glycerol or a combination thereof.

17. The genetically engineered bacterium according to claim 15, wherein the carbon source is selected from glucose and glycerol or a combination thereof.

18. (canceled)

19. The genetically engineered b bacterium according claim 14, wherein the genetically engineered bacterium does not comprise a deletion of glpD gene.

20. The genetically engineered bacterium according to claim 1, further comprising a deletion of glpD gene.

21. The genetically engineered bacterium according to claim 20, wherein the genetically engineered bacterium comprises a deletion of ndh gene, nuoEFG genes, dld gene, and glpD gene.

22. The genetically engineered bacterium according to claim 20, wherein the genetically engineered bacterium comprises a deletion of ndh gene, nuoEFG genes, dld gene, glpD gene, mqo gene, poxB gene, sdhABCD genes, glcDEF genes, lhgO gene, putA gene, lldD gene, dadA gene, kefF gene, mdaB-ygiN gene, glpABC genes, wrbA gene, yieF gene, and fadE gene.

23. The genetically engineered bacterium according to claim 20, wherein the bacterium is genetically engineered to produce D-lactate from a carbon source in presence of oxygen.

24. The genetically engineered bacterium according to claim 23, wherein the carbon source is selected from the group comprising or consisting of glucose, fructose, xylose, arabinose, galactose, mannose, sucrose, cellobiose, and hemicellulose, or a combination thereof.

25. (canceled)

26. The genetically engineered bacterium according to claim 22, wherein the genetically engineered bacterium further comprises a deletion of ldhA gene, and wherein the genetically engineered bacterium further comprises an engineered metabolic pathway for converting pyruvate to isobutanol, wherein said engineered metabolic pathway comprises AlsS, IlvC, IlvD, KivD and SdhA.

27. The genetically engineered bacterium according to claim 26 further comprising a deletion of gpsA gene.

28. The genetically engineered bacterium according to claim 26, wherein the genetically engineered bacterium is genetically engineered to produce isobutanol and/or ethanol, preferably isobutanol from a carbon source in presence of oxygen.

29. The genetically engineered bacterium according to claim 28, wherein the carbon source is selected from the group comprising or consisting of glucose, fructose, xylose, arabinose, galactose, mannose, sucrose, cellobiose, hemicellulose, and glycerol or a combination thereof.

30. (canceled)

31. A method for obtaining a fermentation product comprising the following steps:
a) providing a genetically engineered bacterium of claim 1;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, hemicellulose, and glycerol or a combination thereof;
wherein the supplement is selected from acetate, pyruvate, and casamino acids (CAA).

32. The method according to claim 31, wherein step a) comprises:
a) providing the genetically engineered bacterium of claim 1; and
wherein the fermentation product is D-lactate.

33. The method according to claim 31, wherein step a) comprises:
a) providing the genetically engineered bacterium of claim 14; and
wherein the fermentation product is isobutanol and/or ethanol, preferably isobutanol.

34. The method according to claim 31, wherein the carbon source is selected from glucose and glycerol or a combination thereof.

35. (canceled)

36. A method for obtaining a fermentation product comprising the following steps:
a) providing a genetically engineered bacterium according to claim 20;
b) providing a culture medium comprising a carbon source and optionally a supplement;
c) culturing the genetically engineered bacterium under aerobic conditions in the culture medium of b),
wherein the carbon source is selected from the group comprising or consisting of glucose, fructose, xylose, arabinose, galactose, mannose, sucrose, cellobiose, and hemicellulose, or a combination thereof;
wherein the supplement is selected from acetate, pyruvate, and casamino acids (CAA).

37. The method according to claim 31, wherein step a) comprises:
a) providing the genetically engineered bacterium of claim 20; and
wherein the fermentation product is D-lactate.

38. The method according to claim 31, wherein step a) comprises:
a) providing the genetically engineered bacterium of claim 26; and
wherein the fermentation product is isobutanol and/or ethanol, preferably isobutanol.

39. (canceled)


SEQUENCE LISTING (100 sequences)

SEQ ID NO: 1 - DNA, 72 bp, Escherichia coli
  source 1..72: mol_type = other DNA
taataaccattaattaacaattggttaataaatttaagggggtcacgttgaattaaccctcactaaagggcg

SEQ ID NO: 2 - DNA, 73 bp, Escherichia coli
  source 1..73: mol_type = other DNA
tttatgccacatccgccagtgtacgtcgattaatgcaacttcaaacgcggtaatacgactcactatagggctc

SEQ ID NO: 3 - DNA, 23 bp, Escherichia coli
  source 1..23: mol_type = other DNA
gcgaagaacattttcattgctgt

SEQ ID NO: 4 - DNA, 20 bp, Escherichia coli
  source 1..20: mol_type = other DNA
aggatgcctgatacaacgcg

SEQ ID NO: 5 - DNA, 20 bp, Escherichia coli
  source 1..20: mol_type = other DNA
ggttacccatcaggctaccg

SEQ ID NO: 6 - DNA, 20 bp, Escherichia coli
  source 1..20: mol_type = other DNA
cacagctggggcataagctg

SEQ ID NO: 7 - DNA, 70 bp, Escherichia coli
  source 1..70: mol_type = other DNA
gatctcaatcagttccggtgatatccaactcattgttgtgcctccttgaggtgtaggctggagctgcttc

SEQ ID NO: 8 - DNA, 70 bp, Escherichia coli
  source 1..70: mol_type = other DNA
tatctgggcagtatcgattttgttatgtcagatgtggaccgctaattatgcatatgaatatcctccttag

SEQ ID NO: 9 - DNA, 26 bp, Escherichia coli
  source 1..26: mol_type = other DNA
cggtgccagggtaaagatgacgcgat

SEQ ID NO: 10 - DNA, 26 bp, Escherichia coli
  source 1..26: mol_type = other DNA
tcgaaaccctgatcacccacttcctg

SEQ ID NO: 11 - DNA, 20 bp, Escherichia coli
  source 1..20: mol_type = other DNA
cgatcgagcacgagatgcac

SEQ ID NO: 12 - DNA, 20 bp, Escherichia coli
  source 1..20: mol_type = other DNA
cgatttcaaccggcagcgtg

SEQ ID NO: 13 - DNA, 70 bp, Escherichia coli
  source 1..70: mol_type = other DNA
cttttctgcgatacaatgcctttacgttatgtaacggagagttcggcatggtgtaggctggagctgcttc

SEQ ID NO: 14 - DNA, 70 bp, Escherichia coli
  source 1..70: mol_type = other DNA
ccggatgatcatccggcttttttacatcatcagaaatgccagtaactcatcatatgaatatcctccttag

SEQ ID NO: 15 - DNA, 22 bp, Escherichia coli
  source 1..22: mol_type = other DNA
ttttactgcgagttggcgagct

SEQ ID NO: 16 - DNA, 22 bp, Escherichia coli
  source 1..22: mol_type = other DNA
gcagggcgaagggtaatcagag

SEQ ID NO: 17 - DNA, 18 bp, Escherichia coli
  source 1..18: mol_type = other DNA
gctggcggcgatcaaaga

SEQ ID NO: 18 - DNA, 29 bp, Escherichia coli
  source 1..29: mol_type = other DNA
gcggttgcacctttcaaaaatgttaactg

SEQ ID NO: 19 - DNA, 20 bp, Escherichia coli
  source 1..20: mol_type = other DNA
gcagcggtattcacactcag

SEQ ID NO: 20 - DNA, 20 bp, Escherichia coli
  source 1..20: mol_type = other DNA
cacaccagccattcctcgac

SEQ ID NO: 21 - DNA, 70 bp, Escherichia coli
  source 1..70: mol_type = other DNA
gaatgactttagttttcattttgttattccttttcaagggcttgttctacgtgtaggctggagctgcttc

SEQ ID NO: 22 - DNA, 72 bp, Escherichia coli
  source 1..72: mol_type = other DNA
cgcgcaaaaatcagctgccacacaacacaacaaagcgaagcctactcatgggaattagccatggtccatatg

SEQ ID NO: 23 - DNA, 22 bp, Escherichia coli
  source 1..22: mol_type = other DNA
cgcagtcacgaacgcggtacgt

SEQ ID NO: 24 - DNA, 25 bp, Escherichia coli
  source 1..25: mol_type = other DNA
cacacagtcgacgttccgagggaag

SEQ ID NO: 25 - DNA, 20 bp, Escherichia coli
  source 1..20: mol_type = other DNA
ggataacctgtcgatccgcg

SEQ ID NO: 26 - DNA, 20 bp, Escherichia coli
  source 1..20: mol_type = other DNA
gtgacgcacagaggtacgac

SEQ ID NO: 27 - DNA, 22 bp, Escherichia coli
  source 1..22: mol_type = other DNA
ttcttcctttgttgcccgacgt

SEQ ID NO: 28 - DNA, 22 bp, Escherichia coli
  source 1..22: mol_type = other DNA
tagtgatggacgcgtttggcaa

SEQ ID NO: 29 - DNA, 20 bp, Escherichia coli
  source 1..20: mol_type = other DNA
gaacttgctcgtctggtggg

SEQ ID NO: 30 - DNA, 20 bp, Escherichia coli
  source 1..20: mol_type = other DNA
gaacttgctcgtctggtggg

SEQ ID NO: 31 - DNA, 20 bp, Escherichia coli
  source 1..20: mol_type = other DNA
gccgcatccaacatctaacg

SEQ ID NO: 32 - DNA, 16 bp, Escherichia coli
  source 1..16: mol_type = other DNA
cggactgctgccgtca

SEQ ID NO: 33 - DNA, 20 bp, Escherichia coli
  source 1..20: mol_type = other DNA
cggtttcaactgcggtttcg

SEQ ID NO: 34 - DNA, 20 bp, Escherichia coli
  source 1..20: mol_type = other DNA
ggaactgaactacaccccgc

SEQ ID NO: 35 - DNA, 25 bp, Escherichia coli
  source 1..25: mol_type = other DNA
gacgccatattgccgcagagtcagg

SEQ ID NO: 36 - DNA, 21 bp, Escherichia coli
  source 1..21: mol_type = other DNA
gtcgcggcctggcggacaatg

SEQ ID NO: 37 - DNA, 21 bp, Escherichia coli
  source 1..21: mol_type = other DNA
cattgggtaactgcaggccgc

SEQ ID NO: 38 - DNA, 25 bp, Escherichia coli
  source 1..25: mol_type = other DNA
cgagttgtcatactgggaagtggtg

SEQ ID NO: 39 - DNA, 20 bp, Escherichia coli
  source 1..20: mol_type = other DNA
agtgcctcctttctctccca

SEQ ID NO: 40 - DNA, 20 bp, Escherichia coli
  source 1..20: mol_type = other DNA
gtcgtctccgtaaacgtcgt

SEQ ID NO: 41 - DNA, 21 bp, Escherichia coli
  source 1..21: mol_type = other DNA
ggttaatcggcttctcgtcag

SEQ ID NO: 42 - DNA, 20 bp, Escherichia coli
  source 1..20: mol_type = other DNA
gaacttgctcgtctggtggg

SEQ ID NO: 43 - DNA, 25 bp, Escherichia coli
  source 1..25: mol_type = other DNA
cagcaatatgacgatccgcgggcag

SEQ ID NO: 44 - DNA, 30 bp, Escherichia coli
  source 1..30: mol_type = other DNA
ctcggcaaagtgcagaatagattcggcatc

SEQ ID NO: 45 - DNA, 20 bp, Escherichia coli
  source 1..20: mol_type = other DNA
ctcctgccattccagcagac

SEQ ID NO: 46 - DNA, 20 bp, Escherichia coli
  source 1..20: mol_type = other DNA
gcatccgtatccgcatcatt

SEQ ID NO: 47 - DNA, 26 bp, Escherichia coli
  source 1..26: mol_type = other DNA
gtcgtacccggcgacttcgaaaatgg

SEQ ID NO: 48 - DNA, 37 bp, Escherichia coli
  source 1..37: mol_type = other DNA
ggttgtgaaatttgcactctgaaaaggacgtcttatc

SEQ ID NO: 49 - DNA, 21 bp, Escherichia coli
  source 1..21: mol_type = other DNA
catattgaaacgatggcacgc

SEQ ID NO: 50 - DNA, 20 bp, Escherichia coli
  source 1..20: mol_type = other DNA
caatagacagttcttcctgg

SEQ ID NO: 51 - DNA, 22 bp, Escherichia coli
  source 1..22: mol_type = other DNA
aggcggtacttatactcccgca

SEQ ID NO: 52 - DNA, 22 bp, Escherichia coli
  source 1..22: mol_type = other DNA
aaaagcgagaagtacgggcagg

SEQ ID NO: 53 - DNA, 20 bp, Escherichia coli
  source 1..20: mol_type = other DNA
gctttctgatgtctcgatgg

SEQ ID NO: 54 - DNA, 20 bp, Escherichia coli
  source 1..20: mol_type = other DNA
ctttctccggcaactttacc

SEQ ID NO: 55 - DNA, 27 bp, Escherichia coli
  source 1..27: mol_type = other DNA
cggatattcacagccgcgccaacaatc

SEQ ID NO: 56 - DNA, 31 bp, Escherichia coli
  source 1..31: mol_type = other DNA
gtgcgtaatcatcgttgcgctgctgtttatc

SEQ ID NO: 57 - DNA, 20 bp, Escherichia coli
  source 1..20: mol_type = other DNA
caccaaatgcggtcaattgc

SEQ ID NO: 58 - DNA, 21 bp, Escherichia coli
  source 1..21: mol_type = other DNA
catttaatggcatggttgcac

SEQ ID NO: 59 - DNA, 70 bp, Escherichia coli
  source 1..70: mol_type = other DNA
gtcaggatggccgggcaaacaaggcaggattaaatccctggctgcagaatcatatgaatatcctccttag

SEQ ID NO: 60 - DNA, 29 bp, Escherichia coli
  source 1..29: mol_type = other DNA
cctgcatcggtgagtcagagtaagatcag

SEQ ID NO: 61 - DNA, 29 bp, Escherichia coli
  source 1..29: mol_type = other DNA
cctgcatcggtgagtcagagtaagatcag

SEQ ID NO: 62 - DNA, 26 bp, Escherichia coli
  source 1..26: mol_type = other DNA
ggttgcaagagaaaggcgacatggcg

SEQ ID NO: 63 - DNA, 21 bp, Escherichia coli
  source 1..21: mol_type = other DNA
gtatagcggggaacatcaggc

SEQ ID NO: 64 - DNA, 20 bp, Escherichia coli
  source 1..20: mol_type = other DNA
cgcccactccaatggtcaac

SEQ ID NO: 65 - DNA, 22 bp, Escherichia coli
  source 1..22: mol_type = other DNA
accgcctctttttcctgcaact

SEQ ID NO: 66 - DNA, 22 bp, Escherichia coli
  source 1..22: mol_type = other DNA
aatttttatgctggcgctgggc

SEQ ID NO: 67 - DNA, 20 bp, Escherichia coli
  source 1..20: mol_type = other DNA
gatagggggcggcatcaatg

SEQ ID NO: 68 - DNA, 20 bp, Escherichia coli
  source 1..20: mol_type = other DNA
cggcaccacaatatggctgc

SEQ ID NO: 69 - DNA, 22 bp, Escherichia coli
  source 1..22: mol_type = other DNA
catgcgcggtttcttcgatgtc

SEQ ID NO: 70 - DNA, 22 bp, Escherichia coli
  source 1..22: mol_type = other DNA
tgcggtgtcgtttcagagtgag

SEQ ID NO: 71 - DNA, 20 bp, Escherichia coli
  source 1..20: mol_type = other DNA
gcattgcaggtgtggatcgt

SEQ ID NO: 72 - DNA, 20 bp, Escherichia coli
  source 1..20: mol_type = other DNA
cactatatggatggtggtgc

SEQ ID NO: 73 - DNA, 70 bp, Escherichia coli
  source 1..70: mol_type = other DNA
tttttactatggctaaatggtaaaaaacgaacttcagagggataacaatggtgtaggctggagctgcttc

SEQ ID NO: 74 - DNA, 70 bp, Escherichia coli
  source 1..70: mol_type = other DNA
cacatgcttaccgggcatcagaaaggagtttaagccagcgcctgggccagcatatgaatatcctccttag

SEQ ID NO: 75 - DNA, 22 bp, Escherichia coli
  source 1..22: mol_type = other DNA
cgtttcttgagttgccgcgatg

SEQ ID NO: 76 - DNA, 22 bp, Escherichia coli
  source 1..22: mol_type = other DNA
agtttcagcctttgcgagacgt

SEQ ID NO: 77 - DNA, 22 bp, Escherichia coli
  source 1..22: mol_type = other DNA
gatctgggggcaacacattgcc

SEQ ID NO: 78 - DNA, 20 bp, Escherichia coli
  source 1..20: mol_type = other DNA
gcagcggtttcagcggtaac

SEQ ID NO: 79 - DNA, 70 bp, Escherichia coli
  source 1..70: mol_type = other DNA
gcccgtagtccccagggaataataagaacagcatgtgggcgttattcatggtgtaggctggagctgcttc

SEQ ID NO: 80 - DNA, 70 bp, Escherichia coli
  source 1..70: mol_type = other DNA
acgcttgcgcgtcttatcaggcctacggtttacgcattacgttgcaacaacatatgaatatcctccttag

SEQ ID NO: 81 - DNA, 19 bp, Escherichia coli
  source 1..19: mol_type = other DNA
tcccgacgtctccaggtaa

SEQ ID NO: 82 - DNA, 20 bp, Escherichia coli
  source 1..20: mol_type = other DNA
ttgtctacgtagtggctcgc

SEQ ID NO: 83 - DNA, 20 bp, Escherichia coli
  source 1..20: mol_type = other DNA
gttgtgattggtgccggtgg

SEQ ID NO: 84 - DNA, 20 bp, Escherichia coli
  source 1..20: mol_type = other DNA
gcgtcatggattccgactct

SEQ ID NO: 85 - DNA, 20 bp, Escherichia coli
  source 1..20: mol_type = other DNA
tcgtccagccgaaagacttc

SEQ ID NO: 86 - DNA, 20 bp, Escherichia coli
  source 1..20: mol_type = other DNA
gtccagccattccccgttaa

SEQ ID NO: 87 - DNA, 24 bp, Escherichia coli
  source 1..24: mol_type = other DNA
gcaagatgctggttgccacgtccg

SEQ ID NO: 88 - DNA, 20 bp, Escherichia coli
  source 1..20: mol_type = other DNA
gcattgcaggtgtggatcgt

SEQ ID NO: 89 - DNA, 70 bp, Escherichia coli
  source 1..70: mol_type = other DNA
ttgcagcagcaggctggcgaaggtactgaagaacatcaggatgcctgatggtgtaggctggagctgcttc

SEQ ID NO: 90 - DNA, 70 bp, Escherichia coli
  source 1..70: mol_type = other DNA
gtcatagcggtaacaaaggttccctggggttagtggctgctgcgctcgtccatatgaatatcctccttag

SEQ ID NO: 91 - DNA, 24 bp, Escherichia coli
  source 1..24: mol_type = other DNA
ggtattttctccatcgcgggtatc

SEQ ID NO: 92 - DNA, 22 bp, Escherichia coli
  source 1..22: mol_type = other DNA
atgtagctcagtgcactgccaa

SEQ ID NO: 93 - DNA, 22 bp, Escherichia coli
  source 1..22: mol_type = other DNA
ctcggttattggcatttcaacg

SEQ ID NO: 94 - DNA, 22 bp, Escherichia coli
  source 1..22: mol_type = other DNA
catcaccctggcaagaaatggc

SEQ ID NO: 95 - DNA, 72 bp, Escherichia coli
  source 1..72: mol_type = other DNA
ctcccctggaatgcaggggagcggcaagattaaaccagttcgttcgggcaaattaaccctcactaaagggcg

SEQ ID NO: 96 - DNA, 73 bp, Escherichia coli
  source 1..73: mol_type = other DNA
tatttttagtagcttaaatgtgattcaacatcactggagaaagtcttatgtaatacgactcactatagggctc

SEQ ID NO: 97 - DNA, 24 bp, Escherichia coli
  source 1..24: mol_type = other DNA
acagtttctgactcaggactattt

SEQ ID NO: 98 - DNA, 24 bp, Escherichia coli
  source 1..24: mol_type = other DNA
tttcaatatcgccatagctttcaa

SEQ ID NO: 99 - DNA, 22 bp, Escherichia coli
  source 1..22: mol_type = other DNA
cagcgtagtctgagaaatactg

SEQ ID NO: 100 - DNA, 22 bp, Escherichia coli
  source 1..22: mol_type = other DNA
cgccgtttatagcacaaaacag